SEQUENCE LISTING (31 sequences)

SEQ ID NO: 1 - protein, 329 aa, Artificial Sequence
  Synthetic Construct
MTDVRFRIIGTGAYVPERIVSNDEVGAPAGVDDDWITRKTGIRQRRWAADDQATSDLATAAGRAALKAAGITPEQLTVIAVATSTPDRPQPPTAAYVQHHLGATGTAAFDVNAVCSGTVFALSSVAGTLVYRGGYALVIGADLYSRILNPADRKTVVLFGDGAGAMVLGPTSTGTGPIVRRVALHTFGGLTDLIRVPAGGSRQPLDTDGLDAGLQYFAMDGREVRRFVTEHLPQLIKGFLHEAGVDAADISHFVPHQANGVMLDEVFGELHLPRATMHRTVETYGNTGAASIPITMDAAVRAGSFRPGELVLLAGFGGGMAASFALIEW

SEQ ID NO: 2 - DNA, 125 bp, Artificial Sequence
  Synthetic Construct
aattcatataaaaaacatacagataaccatctgcggtgataaattatctctggcggtgttgacataaataccactggcggtgatactgagcacatcagcaggacgcactgaccaccatgaaggtg

SEQ ID NO: 3 - DNA, 22 bp, Artificial Sequence
  Synthetic Construct
agctggnaccatangggaatnc

SEQ ID NO: 4 - DNA, 28 bp, Artificial Sequence
  Synthetic Construct
atttaancgatacantaatanatacctc

SEQ ID NO: 5 - DNA, 30 bp, Artificial Sequence
  Synthetic Construct
atgagcanttctancggtanccatgatctt

SEQ ID NO: 6 - DNA, 31 bp, Artificial Sequence
  Synthetic Construct
aacgtgcntcataganacgtntatggtcgtt

SEQ ID NO: 7 - DNA, 61 bp, Artificial Sequence
  Synthetic Construct
atatnaatgtancgattaaanaaggaggaataaaccatgaccgaaactcatgcaattgctg

SEQ ID NO: 8 - DNA, 82 bp, Artificial Sequence
  Synthetic Construct
ataccganagaaangctcantggtatatcctcctagtgcgtctagattacgcaccaactttcgcggttttgttatcacgttc

SEQ ID NO: 9 - DNA, 72 bp, Artificial Sequence
  Synthetic Construct
aacgnatctangaagcacgtnaaagatctcgcactaggaggatataccaatgaccgacgtgcgctttcggat

SEQ ID NO: 10 - DNA, 48 bp, Artificial Sequence
  Synthetic Construct
aattcccanatggnaccagcngcagtcaccattcaatcaacgcgaagg

SEQ ID NO: 11 - DNA, 50 bp, Artificial Sequence
  Synthetic Construct
cggtgaacgctctcctgagtagcatgagattatcaaaaaggatcttcacc

SEQ ID NO: 12 - DNA, 50 bp, Artificial Sequence
  Synthetic Construct
gggacagctgatagaaacagaagccaaatatgtatccgctcatgagacaa

SEQ ID NO: 13 - DNA, 50 bp, Artificial Sequence
  Synthetic Construct
ttgtctcatgagcggatacatatttggcttctgtttctatcagctgtccc

SEQ ID NO: 14 - DNA, 50 bp, Artificial Sequence
  Synthetic Construct
ggtgaagatcctttttgataatctcatgctactcaggagagcgttcaccg

SEQ ID NO: 15 - DNA, 22 bp, Artificial Sequence
  Synthetic Construct
gcaattccccttctacgctggg

SEQ ID NO: 16 - DNA, 23 bp, Artificial Sequence
  Synthetic Construct
ctcgaccttcacgttgttacgcc

SEQ ID NO: 17 - DNA, 73 bp, Artificial Sequence
  Synthetic Construct
tatttaatttttaatcatctaatttgacaatcattcaacaaagttgttacaattaaccctcactaaagggcgg

SEQ ID NO: 18 - DNA, 147 bp, Artificial Sequence
  Synthetic Construct
tcaacagctgtatccccgttgagggtgagttttgcttttgtatcagccatatattccaccagctatttgttagtgaataaaagtggttgaattatttgctcaggatgtggcathgtcaagggctaatacgactcactatagggctcg

SEQ ID NO: 19 - DNA, 25 bp, Artificial Sequence
  Synthetic Construct
ggcagtataggctgttcacaaaatc

SEQ ID NO: 20 - DNA, 23 bp, Artificial Sequence
  Synthetic Construct
cttgacccagcgtgcctttcagc

SEQ ID NO: 21 - DNA, 22 bp, Artificial Sequence
  Synthetic Construct
gcttatatgcgtgctatcagcg

SEQ ID NO: 22 - DNA, 7933 bp, Artificial Sequence
  Synthetic Construct
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggcatctgctactaactcatctgtaattgttgctggtgcacgtactccgatgggtcgcctgctgggttccctgaaatctttctctggtgcggatctgggtggcttcgcgatcaaagcagcactggaccgcgcaggcattggcggcgaccaggtccagtacgttatcatgggtcaggtgttgcaggcaggtgcgggtcagattccggctcgtcaggcagcggttaaagctggcattccgatgaacgtaccagctctgaccatcaacaaagtgtgcctgtccggtctggacgcgatcgcgctggccgatcaactgatccgcgctggtgaatttgacattgttgtagcaggcggccaggaatctatgactaacgccccgcaccttctgccgaagtctcgtgaaggttataagtacggcgctatcgagatgctcgatgcaatggcttacgacggcctgactgacccgtgggaaaacattccgatgggccagagcactgagaaacacaacacccgtctgggtatcggtcgtgaagttcaggatgagatcgcggcgctgtctcaccagcgtgctgctgcggcccagaaaaatggcctcttcgaagctgaaatcaccccgatcgaaattccgcagcgtaaaggtgaaccggttgtattcagcaaagacgaaggcattcgcgcggaaaccactgctgaatccctcggcaaactgcgtccggctttcactaaggacggtactatcactgcgggcaccgcgtctcagatttccgatggtgcggcagccgttgttgtcatgtcgaaagcgaaagcgttggaactgggtctggattggatcgctgaaattggtgcccacggcaacgtggcaggcccggacaattctctgcaatcccaaccatcaaacgcaatcctgcacgcactgaagaaagaaggtcttgaagtagaagacctggacctgatcgagatcaacgaagcgtttgccgcggttgcccaccaatctatgaaagacctgggtgtctccaccgaaaaggttaacgtgaacggtggcgcaatcgccctgggccatccgattggcatgtccggtgctcgtctggttctgcacctggcactggagttgaagcgtcgtggtggtggcgtaggtgcagccgccctgtgtggtggcggtggtcagggcgatgcactgatcgtacgtgtcccgaaagcgtaagccttcttaaggtagctcagatacccttataagctttacaaggaggaaaaaaacatgagcatttctatcggtatccatgatctttcgttcgcgactaccgagttcgttctgcctcatacggctctggccgagtataatggtactgaaattggtaaatatcacgttggcatcggtcaacagagcatgagcgtacctgcggcggatgaggacatcgtgactatggccgcgaccgccgcacgtccgatcattgaacgcaacggtaaatcccgtatccgcactgtggtgttcgctacggagtcctccattgatcaagctaaagcgggtggcgtatacgtacattctctgctgggcctggaatccgcgtgccgtgtagttgaactgaaacaggcatgctacggtgcgacggcagcattgcagttcgctattggtctggttcgccgcgatccggcgcagcaggtgctggtgatcgcaagcgacgtttctaaatacgagttggattcgccgggtgaggcaacccagggcgcagcggccgttgctatgctggttggcgcagatcctgcactgcttcgcattgaagaaccgtccggcctcttcaccgcggacgttatggatttctggcgcccgaactacctgaccaccgccctggtagatggccaggagtccatcaacgcctacctgcaagcagttgaaggtgcgtggaaggactacgcagagcaggacggtcgtagccttgaagagtttgcagctttcgtttaccaccagccgttcactaaaatggcgtacaaagctcaccgccacctgctgaacttcaacggttacgacactgacaaagatgcgattgaaggcgccctgggtcaaactaccgcttacaataatgtgatcggtaactcctacactgcgtcagtttatctgggcctggcggcgcttctggatcaggcggacgacctgaccggtcgttctatcggcttcctgtcttacggctccggtagcgtagcggagtttttctcgggcaccgttgttgctggttaccgtgaacgtctgcgtactgaagcgaaccaggaagctatcgcacgccgtaaatctgtcgattacgcgacttatcgtgaactgcatgagtacacgctcccgtctgatggtggtgatcacgccacgccggttcagaccaccggtccattccgtctggcaggtatcaacgaccataaacgtatctatgaagcacgttaagcctgacttaaggtagctgctttcgcccttatgagctctacaaggaggaaaaaaacatgaccgaaactcatgcaattgctggtgtaccgatgcgttgggttggcccgctgcgtatctctggcaacgtagcagaaaccgaaactcaggttccgctggcaacctacgaatctccgctgtggccgtctgtgggtcgtggtgcgaaggtatcccgtctgaccgaaaagggtatcgttgcaaccctcgttgacgaacgtatgacccgtagcgtcatcgtagaagccaccgacgcccagaccgcttatatggctgcacagaccattcatgctcgcattgatgaactgcgcgaagtggttcgtggttgttcccgtttcgcgcagctgattaatatcaaacatgaaattaacgccaacttgctgttcatccgctttgaatttaccaccggtgacgcatctggccataacatggccaccctggcgtctgacgttctgttgggtcacctcctggaaaccatcccaggcatctcctacggctccatttctggcaactactgcactgacaagaaagcaactgccattaacggtatcctgggtcgcggcaaaaacgttatcaccgaattgctggtgccgcgcgatgttgtagaaaataacctgcacaccaccgctgcgaaaatcgttgaactgaacatccgtaaaaacctgctgggcactctcctggcaggcggcatccgttctgcgaacgcgcattttgcaaatatgctgctgggtttctacctggcaactggccaggacgcagcaaacatcgttgagggtagccagggcgttgtaatggcggaagatcgtgacggtgatctgtacttcgcgtgcacccttccgaatctgatcgtgggtactgttggcaacggtaagggcctgggttttgttgaaacgaatctggcccgtctgggctgtcgcgcggatcgcgaaccgggcgaaaatgcgcgccgtctggccgttatcgccgctgccaccgtgctgtgtggtgaactgtccctgttggccgcgcagaccaaccctggtgaactgatgcgtgcacatgttcagctggaacgtgataacaaaaccgcgaaagttggtgcgtaaagatctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 23 - DNA, 6693 bp, Artificial Sequence
  Synthetic Construct
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatccggatatagttcctcctttcagcaaaaaacccctcaagacccgtttagaggccccaaggggttatgctagttattgctcagcggtggcagcagccaactcagcttcctttcgggctttgttagcagccggatccttatcaccattcaatcaacgcgaaggaagctgccatgcccccgccgaaacctgccaacagaacgagttcgcctgggcgaaaggacccagcgcgcactgctgcatccatagtgataggtatggacgcagcgcctgtattaccgtatgtttcaactgtacggtgcatagtggcacgagggagatgcaattcgccgaacacttcatccagcataacgccgttcgcctggtgtgggacaaagtgcgaaatgtctgccgcatccactcctgcttcatgcagaaaccccttaatcagctgcggaaggtgctctgtcacgaaacgccgcacttcgcgaccatccattgcaaagtattgaagaccggcatcaagcccgtctgtgtcaagaggttgccgtgaaccacctgctggcacgcggatcagatccgtcagtcctccgaaggtatgaagagcgacccggcgcacgattggcccggttccagtggacgtcgggcctaataccatcgcaccagccccgtcaccaaaaagtaccactgttttacgatccgcagggttcaggatacgcgaatacaggtccgcccctattaccagagcgtatccgccgcggtaaaccaacgtacccgcaactgacgaaagtgcgaaaaccgtcccgctgcacactgcgttcacatcgaatgcggcggtgcctgttgcacccaaatgatgctgaacataagcagcggtagggggctgaggacggtccggggtcgaagtagccaccgcaataacggttaactgctccggagttatgcccgcagccttcagtgccgcacgacccgcggcagtggcaaggtcactagtcgcttgatcatctgccgcccaccgtctctgacgtatacccgtcttgcgtgtaatccaatcatcgtccacaccagccggtgcgccaacttcgtcattactcacgatgcgttccggcacgtacgcaccggtaccaattatccgaaagcgcacgtcggtcatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 24 - DNA, 8364 bp, Artificial Sequence
  Synthetic Construct
ctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcgggaattcgcgttggccgattcattaatgcagattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgaccgaaactcatgcaattgctggtgtaccgatgcgttgggttggcccgctgcgtatctctggcaacgtagcagaaaccgaaactcaggttccgctggcaacctacgaatctccgctgtggccgtctgtgggtcgtggtgcgaaggtatcccgtctgaccgaaaagggtatcgttgcaaccctcgttgacgaacgtatgacccgtagcgtcatcgtagaagccaccgacgcccagaccgcttatatggctgcacagaccattcatgctcgcattgatgaactgcgcgaagtggttcgtggttgttcccgtttcgcgcagctgattaatatcaaacatgaaattaacgccaacttgctgttcatccgctttgaatttaccaccggtgacgcatctggccataacatggccaccctggcgtctgacgttctgttgggtcacctcctggaaaccatcccaggcatctcctacggctccatttctggcaactactgcactgacaagaaagcaactgccattaacggtatcctgggtcgcggcaaaaacgttatcaccgaattgctggtgccgcgcgatgttgtagaaaataacctgcacaccaccgctgcgaaaatcgttgaactgaacatccgtaaaaacctgctgggcactctcctggcaggcggcatccgttctgcgaacgcgcattttgcaaatatgctgctgggtttctacctggcaactggccaggacgcagcaaacatcgttgagggtagccagggcgttgtaatggcggaagatcgtgacggtgatctgtacttcgcgtgcacccttccgaatctgatcgtgggtactgttggcaacggtaagggcctgggttttgttgaaacgaatctggcccgtctgggctgtcgcgcggatcgcgaaccgggcgaaaatgcgcgccgtctggccgttatcgccgctgccaccgtgctgtgtggtgaactgtccctgttggccgcgcagaccaaccctggtgaactgatgcgtgcacatgttcagctggaacgtgataacaaaaccgcgaaagttggtgcgtaatctagacgcactaggaggatataccaatgagcatttctatcggtatccatgatctttcgttcgcgactaccgagttcgttctgcctcatacggctctggccgagtataatggtactgaaattggtaaatatcacgttggcatcggtcaacagagcatgagcgtacctgcggcggatgaggacatcgtgactatggccgcgaccgccgcacgtccgatcattgaacgcaacggtaaatcccgtatccgcactgtggtgttcgctacggagtcctccattgatcaagctaaagcgggtggcgtatacgtacattctctgctgggcctggaatccgcgtgccgtgtagttgaactgaaacaggcatgctacggtgcgacggcagcattgcagttcgctattggtctggttcgccgcgatccggcgcagcaggtgctggtgatcgcaagcgacgtttctaaatacgagttggattcgccgggtgaggcaacccagggcgcagcggccgttgctatgctggttggcgcagatcctgcactgcttcgcattgaagaaccgtccggcctcttcaccgcggacgttatggatttctggcgcccgaactacctgaccaccgccctggtagatggccaggagtccatcaacgcctacctgcaagcagttgaaggtgcgtggaaggactacgcagagcaggacggtcgtagccttgaagagtttgcagctttcgtttaccaccagccgttcactaaaatggcgtacaaagctcaccgccacctgctgaacttcaacggttacgacactgacaaagatgcgattgaaggcgccctgggtcaaactaccgcttacaataatgtgatcggtaactcctacactgcgtcagtttatctgggcctggcggcgcttctggatcaggcggacgacctgaccggtcgttctatcggcttcctgtcttacggctccggtagcgtagcggagtttttctcgggcaccgttgttgctggttaccgtgaacgtctgcgtactgaagcgaaccaggaagctatcgcacgccgtaaatctgtcgattacgcgacttatcgtgaactgcatgagtacacgctcccgtctgatggtggtgatcacgccacgccggttcagaccaccggtccattccgtctggcaggtatcaacgaccataaacgtatctatgaagcacgttaaagatctcgcactaggaggatataccaatgaccgacgtgcgctttcggataattggtaccggtgcgtacgtgccggaacgcatcgtgagtaatgacgaagttggcgcaccggctggtgtggacgatgattggattacacgcaagacgggtatacgtcagagacggtgggcggcagatgatcaagcgactagtgaccttgccactgccgcgggtcgtgcggcactgaaggctgcgggcataactccggagcagttaaccgttattgcggtggctacttcgaccccggaccgtcctcagccccctaccgctgcttatgttcagcatcatttgggtgcaacaggcaccgccgcattcgatgtgaacgcagtgtgcagcgggacggttttcgcactttcgtcagttgcgggtacgttggtttaccgcggcggatacgctctggtaataggggcggacctgtattcgcgtatcctgaaccctgcggatcgtaaaacagtggtactttttggtgacggggctggtgcgatggtattaggcccgacgtccactggaaccgggccaatcgtgcgccgggtcgctcttcataccttcggaggactgacggatctgatccgcgtgccagcaggtggttcacggcaacctcttgacacagacgggcttgatgccggtcttcaatactttgcaatggatggtcgcgaagtgcggcgtttcgtgacagagcaccttccgcagctgattaaggggtttctgcatgaagcaggagtggatgcggcagacatttcgcactttgtcccacaccaggcgaacggcgttatgctggatgaagtgttcggcgaattgcatctccctcgtgccactatgcaccgtacagttgaaacatacggtaatacaggcgctgcgtccatacctatcactatggatgcagcagtgcgcgctgggtcctttcgcccaggcgaactcgttctgttggcaggtttcggcgggggcatggcagcttccttcgcgttgattgaatggtga

SEQ ID NO: 25 - DNA, 8807 bp, Artificial Sequence
  Synthetic Construct
cccgtcttactgtcgggaattcgcgttggccgattcattaatgcagattctgaaatgagctgttgacaattaatcatccggctcgtataatgtgtggaattgtgagcggataacaatttcacacaggaaacagcgccgctgagaaaaagcgaagcggcactgctctttacaatttatcagacaatctgtgtgggcactcgaccggaattatcgattaactttattattaaaaattaaagaggtatatattaatgtatcgattaaataaggaggaataaaccatgagcacgtttagtggtttttacaagaaaagtcgccaggagcgcatcgatatcttacgtcagaaccggtccctggcagaagattctctggatattctgtacaaggacgaaaacctgcctgaagctatcgcgggcaaaatggccgaaaaccacttggggacgttcagcctgcccttctcggtactgcctgagctgctggtagatgggcagacatactctgttcctatggtaactgaggagcctagcgtggtagcagccgcctcgttcggggcaaaaattatcgcgaaatccggtggctttacaacaaccatccacaaccgtataatgatcggccaggtagcgttatatgatataagtgaccattctcgcgccacgcaagcaattctggatcacaaggagagtatacttgaaaacgctaaccaagctcatcccagtatagtcaaacgcggcggaggggctagagagcttacagttgagtctaaggatgaatttctgatcgtctaccttcaggtagatgtgcaagaagcaatgggtgcaaacatactgaacaacatgcttgaagccgtgaaagatgatctggaagaactttccaaaggccaggcgcttatgggaatcctcagcaactacgccaccgagtcattaatcacagcacagtgtcatatcgcaatatcaagcctggcgacttcctctgccattgctcaggagaccgcccagaaaattgcactcgcgagcaaattagcgcaagttgacccatatcgtgccgcgacacacaataaaggtatttttaatgggattgacgctgtcgtcattgcagctgggaacgactggcgtgctgttgaggcaggtgctcatgcgtatgccagccgcgatggacaatataaaggcctgagtacctggtcgatcgatggggaacacttagttggctccattacattgccgttgcctatagcttcagttggcggaagtataggcctgaatccgaaggttgccgtcgcatttgacttactgcaacagccaaaagcacgccaattagccagcattattgcctcagtgggtctctgccaaaatttcgctgctctccgggcgctggtaactagtggtattcaggccggtcacatgaagttacatgctaaatcgctggctttactggccggggcggaagaacatgaggtcgaccaactggctcagctgctccgcaaggaaaaacattccaatcttgaaacggctcagaacttactggcaaaaatgcgtgaacattgggaatgggaaagacatccttgatctagacgcactaggaggatataccaatgcgtaaacaagttaacctttcttttgtgcataagggctacattatgaacattggtattgacaagatcggtttcgcagcccccgattatgtgttagacttagcggatttagcccaggcgcgtaatgttgatcctaacaaatttaagatcggtttacttcagagcgagatggccgtcgccccggtgacgcaggatatcatatcgttaggcgccaaagctgccgaggcaattctgaccgaagaggacaaacaaacgatcgatatggttatcgtgggcaccgaaagttccgtcgatcaaagcaaagccgcggcggtgacaatacacggactgttaggcatccagcctttcgctcggtcgattgaaatgaaggaggcttgttatggcgcgactgccggattaagtttggctaagtctcatatcgcccagtttcctgaaagcaaagttttagtcatagcctcggatattgctaaatatggggtagcttccggaggtgaacctactcagggtgcgggagccgtggcaatgcttgtgacggcgaaccctcggattctggtgttgaacaacgataatgtctgccaaacgcgcgatatatacgatttttggcgtccgaattatgacaagtacccacgtgtcgacggcaagttctccaccgaacagtatacagactgcttaactactacattcgattactatcaacagaagacggggaaaaccctgaacgactttgccgcaatgtgcttgcacatccccttctctaaacaaggtctgaaaggcttacaggcgattgcccaagatgaagaaaccctcagccggttaacggagcgcttccaggaagccattgtctacaacaaagtggtggggaatatctataccggcagcattttcttgagtctcctgtctctcctggaaaattcacgggctctggaaacaggagatcagattctgttctacagttacggctctggtgctgtatgcgaaattttctctggacaactggtcgaagggtaccgcaatcacctgcaagagaatcgcctggaacagttgaaccagcgtaccaaactgtccgtcaaggaatacgaacaggtgttttttgaggaaataaccctggatgaaactgggtcctccttggatctcccagaagatcagtccccgttcgcgcttattaaagtcgataaccacaaacgtatctatcgtaaatgaagatctcgcactaggaggatataccaatgaagaaagtcgcgatcgtctctgcttaccggagcgcaatcgggtcctttggcggatcattgaaggacatcgagattgccgatttaggcgcacaggtattggagacggccctcgcctcgaagaacataccggcggatagcgtggacgaagttatcttcggtaacgtactgtccgccggtcaaggtcagaacatagcaagacagatagccatacgtgccgggattccacaaacagcatcagcgtacgccgtaaataaggtgtgtggttcgggtttaaaatcagtgcttctggcggcacagtcgattatgctgggcgacaacgatgtcgtcgttgcaggtggaattgaaatcatgtcccaggcaccgtatttgtcaaaaactagtcgctttgggagtaagttcgggcacatcacccttgaagattccatgctgacggatggtctcactgatgcgtttaatgactaccacatgggcatcactgcggaaaatgtagcggagcattatcaggtaagccgtgccgagcaggacgccttcacttactcatctcaggaaaaagctgccaaggcgatagcagaaggacgttttgtggacgaaatcgttcctatccgtctcaaaaaccgcaagggcgaaacgatattcgcaactgatgagtatcctagactgaccccaattgaaaagttagctaccctccgcccatcttttaagaaggatggaaccgtcacagcagcaaacgcctccggcattaatgacgggtgcgcggtgctgatacttatgtcagacgagaaagcgtccgagttaaacatccagccgctgacatacatagaagcctatgcaacctcgggtctcgatcccgcccttatgggactgggtccgattaccgcctctcaaaaagcccttcagaaattgaacaaaacggttgaagacatcgacctgtttgaattaaatgaagcgttcgctgcacagagtattcccgttgtgaaacagctcgggatcgatccggcgaaagtcaacgttaacggtggggcaatagctttaggtcaccccatcggcgcttcaggtagccgcattcttgtcacgcttatccacgaactcatcaaacaagagaaagaactcggcttatgctcactgtgtattggcggcggtcaggggattagtctgatagtatccaacgctcaaacttcctgactgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggctta

SEQ ID NO: 26 - DNA, 51 bp, Artificial Sequence
  Synthetic Construct
gggcagatctcgcactaggaggatataccaatgaccgacgtgcgctttcgg

SEQ ID NO: 27 - DNA, 37 bp, Artificial Sequence
  Synthetic Construct
tatcctgcagtcaccattcaatcaacgcgaaggaagc

SEQ ID NO: 28 - DNA, 18 bp, Artificial Sequence
  Synthetic Construct
cggcactgaaggctgcgg

SEQ ID NO: 29 - DNA, 18 bp, Artificial Sequence
  Synthetic Construct
ccgcagccttcagtgccg

SEQ ID NO: 30 - DNA, 18 bp, Artificial Sequence
  Synthetic Construct
ggcatggggtcaggtggg

SEQ ID NO: 31 - DNA, 8612 bp, Artificial Sequence
  Synthetic Construct
cccgtcttactgtcgggaattcgcgttggccgattcattaatgcagattctgaaatgagctgttgacaattaatcatccggctcgtataatgtgtggaattgtgagcggataacaatttcacacaggaaacagcgccgctgagaaaaagcgaagcggcactgctctttacaatttatcagacaatctgtgtgggcactcgaccggaattatcgattaactttattattaaaaattaaagaggtatatattaatgtatcgattaaataaggaggaataaaccatgagcacgtttagtggtttttacaagaaaagtcgccaggagcgcatcgatatcttacgtcagaaccggtccctggcagaagattctctggatattctgtacaaggacgaaaacctgcctgaagctatcgcgggcaaaatggccgaaaaccacttggggacgttcagcctgcccttctcggtactgcctgagctgctggtagatgggcagacatactctgttcctatggtaactgaggagcctagcgtggtagcagccgcctcgttcggggcaaaaattatcgcgaaatccggtggctttacaacaaccatccacaaccgtataatgatcggccaggtagcgttatatgatataagtgaccattctcgcgccacgcaagcaattctggatcacaaggagagtatacttgaaaacgctaaccaagctcatcccagtatagtcaaacgcggcggaggggctagagagcttacagttgagtctaaggatgaatttctgatcgtctaccttcaggtagatgtgcaagaagcaatgggtgcaaacatactgaacaacatgcttgaagccgtgaaagatgatctggaagaactttccaaaggccaggcgcttatgggaatcctcagcaactacgccaccgagtcattaatcacagcacagtgtcatatcgcaatatcaagcctggcgacttcctctgccattgctcaggagaccgcccagaaaattgcactcgcgagcaaattagcgcaagttgacccatatcgtgccgcgacacacaataaaggtatttttaatgggattgacgctgtcgtcattgcagctgggaacgactggcgtgctgttgaggcaggtgctcatgcgtatgccagccgcgatggacaatataaaggcctgagtacctggtcgatcgatggggaacacttagttggctccattacattgccgttgcctatagcttcagttggcggaagtataggcctgaatccgaaggttgccgtcgcatttgacttactgcaacagccaaaagcacgccaattagccagcattattgcctcagtgggtctctgccaaaatttcgctgctctccgggcgctggtaactagtggtattcaggccggtcacatgaagttacatgctaaatcgctggctttactggccggggcggaagaacatgaggtcgaccaactggctcagctgctccgcaaggaaaaacattccaatcttgaaacggctcagaacttactggcaaaaatgcgtgaacattgggaatgggaaagacatccttgatctagacgcactaggaggatataccaatgcgtaaacaagttaacctttcttttgtgcataagggctacattatgaacattggtattgacaagatcggtttcgcagcccccgattatgtgttagacttagcggatttagcccaggcgcgtaatgttgatcctaacaaatttaagatcggtttacttcagagcgagatggccgtcgccccggtgacgcaggatatcatatcgttaggcgccaaagctgccgaggcaattctgaccgaagaggacaaacaaacgatcgatatggttatcgtgggcaccgaaagttccgtcgatcaaagcaaagccgcggcggtgacaatacacggactgttaggcatccagcctttcgctcggtcgattgaaatgaaggaggcttgttatggcgcgactgccggattaagtttggctaagtctcatatcgcccagtttcctgaaagcaaagttttagtcatagcctcggatattgctaaatatggggtagcttccggaggtgaacctactcagggtgcgggagccgtggcaatgcttgtgacggcgaaccctcggattctggtgttgaacaacgataatgtctgccaaacgcgcgatatatacgatttttggcgtccgaattatgacaagtacccacgtgtcgacggcaagttctccaccgaacagtatacagactgcttaactactacattcgattactatcaacagaagacggggaaaaccctgaacgactttgccgcaatgtgcttgcacatccccttctctaaacaaggtctgaaaggcttacaggcgattgcccaagatgaagaaaccctcagccggttaacggagcgcttccaggaagccattgtctacaacaaagtggtggggaatatctataccggcagcattttcttgagtctcctgtctctcctggaaaattcacgggctctggaaacaggagatcagattctgttctacagttacggctctggtgctgtatgcgaaattttctctggacaactggtcgaagggtaccgcaatcacctgcaagagaatcgcctggaacagttgaaccagcgtaccaaactgtccgtcaaggaatacgaacaggtgttttttgaggaaataaccctggatgaaactgggtcctccttggatctcccagaagatcagtccccgttcgcgcttattaaagtcgataaccacaaacgtatctatcgtaaatgaagatctcgcactaggaggatataccaatgaccgacgtgcgctttcggataattggtaccggtgcgtacgtgccggaacgcatcgtgagtaatgacgaagttggcgcaccggctggtgtggacgatgattggattacacgcaagacgggtatacgtcagagacggtgggcggcagatgatcaagcgactagtgaccttgccactgccgcgggtcgtgcggcactgaaggctgcgggcataactccggagcagttaaccgttattgcggtggctacttcgaccccggaccgtcctcagccccctaccgctgcttatgttcagcatcatttgggtgcaacaggcaccgccgcattcgatgtgaacgcagtgtgcagcgggacggttttcgcactttcgtcagttgcgggtacgttggtttaccgcggcggatacgctctggtaataggggcggacctgtattcgcgtatcctgaaccctgcggatcgtaaaacagtggtactttttggtgacggggctggtgcgatggtattaggcccgacgtccactggaaccgggccaatcgtgcgccgggtcgctcttcataccttcggaggactgacggatctgatccgcgtgccagcaggtggttcacggcaacctcttgacacagacgggcttgatgccggtcttcaatactttgcaatggatggtcgcgaagtgcggcgtttcgtgacagagcaccttccgcagctgattaaggggtttctgcatgaagcaggagtggatgcggcagacatttcgcactttgtcccacaccaggcgaacggcgttatgctggatgaagtgttcggcgaattgcatctccctcgtgccactatgcaccgtacagttgaaacatacggtaatacaggcgctgcgtccatacctatcactatggatgcagcagtgcgcgctgggtcctttcgcccaggcgaactcgttctgttggcaggtttcggcgggggcatggcagcttccttcgcgttgattgaatggtgactgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggctta

(19) United States
(12) Patent Application Publication
(10) Pub. No.: US 2013/0122562 A1
(43) Pub. Date: May 16, 2013

(54) PRODUCTION OF ISOPRENE, ISOPRENOID PRECURSORS, AND ISOPRENOIDS USING ACETOACETYL-COA SYNTHASE

(71) Applicants: Ilana S. Aldor, Palo Alto, CA (US); Zachary Q. Beck, Palo Alto, CA (US); Michael C. Miller, San Francisco, CA (US); Caroline M. Peres, Palo Alto, CA (US)

(72) Inventors: Ilana S. Aldor, Palo Alto, CA (US); Zachary Q. Beck, Palo Alto, CA (US); Michael C. Miller, San Francisco, CA (US); Caroline M. Peres, Palo Alto, CA (US)

(73) Assignee: Danisco US Inc., Palo Alto, CA (US)

(21) Appl. No.: 13/566,916

(22) Filed: Aug. 3, 2012

Related U.S. Application Data
(60) Provisional application No. 61/515,300, filed on Aug. 4, 2011.

(51) Int. Cl. C12N 15/81; C12N 15/70

(52) U.S. Cl. CPC C12N 15/81; C12N 15/70
USPC 435/167; 435/252.3; 435/254.11; 435/254.2; 435/257.2; 435/252.33; 435/252.32; 435/252.31; 435/252.34; 435/252.35; 435/254.21; 435/254.23; 435/254.22

(57) ABSTRACT

This invention relates to a recombinant microorganism capable of producing isoprene and isoprene production with the use of such recombinant microorganism with good efficiency. In this invention, the acetoacetyl-CoA synthase gene encoding an enzyme capable of synthesizing acetoacetyl-CoA from malonyl-CoA and acetyl-CoA and one or more genes involved in isoprene biosynthesis that enables synthesis of isoprene from acetoacetyl-CoA are introduced into a host microorganism.

### CROSS-REFERENCE TO RELATED APPLICATIONS

This application claims the benefit of U.S. Provisional Application No. 61/515,300 filed Aug. 4, 2011, the disclosures of which are incorporated herein by reference in their entirety.

### INCORPORATION BY REFERENCE

The Sequence Listing submitted in an ASCII text file, in accordance with 37 C.F.R. §1.821(c) and (e), is incorporated by herein by reference. The text file name is “643842003600_Sequence_Listing.txt”, the date of creation of the text file is Aug. 2, 2012, and the size of the ASCII text file in bytes is 58,064.

### FIELD OF THE INVENTION

The present invention relates generally to methods for producing isoprene, isoprenoid precursors, and/or isoprenoids from cultured cells and compositions that include these cultured cells.

### BACKGROUND OF THE INVENTION

The products of the mevalonate-dependent pathway are isopentenyl pyrophosphate (IPP) and dimethylallyl diphosphate (DMAPP). IPP and DMAPP are precursors to isoprene as well as to isoprenoids.

Isoprene (2-methyl-1,3-butadiene) is the critical starting material for a variety of synthetic polymers, most notably synthetic rubbers. Isoprene is naturally produced by a variety of microbial, plant, and animal species. In particular, two pathways have been identified for the biosynthesis of isoprene: the mevalonate (MVA) pathway and the non-mevalonate (DXP) pathway. However, the yield of isoprene from naturally-occurring organisms is commercially unattractive. Isoprene can also be obtained by fractionating petroleum, the purification of this material is expensive and time-consuming. Petroleum cracking of the C5 stream of hydrocarbons produces only about 15% isoprene. About 800,000 tons per year of cis-polyisoprene are produced from the polymerization of isoprene; most of this polyisoprene is used in the tire and rubber industry. Isoprene is also copolymerized for use as a synthetic elastomer in other products such as footwear, mechanical products, medical products, sporting goods, and latex.

Isoprenoids are compounds derived from the isoprenoid precursor molecules IPP and DMAPP. Over 29,000 isoprenoid compounds have been identified and new isoprenoids are being discovered each year. Isoprenoids can be isolated from natural products, such as microorganisms and species of plants that use isoprenoid precursor molecules as a basic building block to form the relatively complex structures of isoprenoids. Isoprenoids are vital to most living organisms and cells, providing a means to maintain cellular membrane fluidity and electron transport. In nature, isoprenoids function in roles as diverse as natural pesticides in plants to contributing to the scents associated with cinnamon, cloves, and ginger. Moreover, the pharmaceutical and chemical communities use isoprenoids as pharmaceuticals, nutraceuticals, flavoring agents, and agricultural pest control agents. Given their importance in biological systems and usefulness in a broad range of applications, isoprenoids have been the focus of much attention by scientists.

Thus, more economical methods for producing isoprene and/or isoprenoids are needed. In particular, methods that produce isoprene and/or isoprenoids from inexpensive, renewable starting materials at rates, titers, and purity that are sufficient to meet the demands of a robust commercial process are desirable.

Such improvements are provided herein by the disclosure of recombinant microorganisms and their use in methods to produce isoprene, isoprenoid precursors, and/or isoprenoids.

Throughout this specification, various patents, patent applications and other types of publications (e.g., journal articles) are referenced. The disclosure of all patents, patent applications, and publications cited herein are hereby incorporated by reference in their entirety for all purposes.

### BRIEF SUMMARY OF THE INVENTION

The invention provides, inter alia, compositions of recombinant microorganisms and methods of making and using these recombinant microorganisms for producing isoprene, isoprenoid precursors and/or isoprenoids. The recombinant microorganisms comprise an enzyme capable of synthesizing acetoacetyl-CoA from malonyl-CoA and acetyl-CoA which then can be used to make isoprene, isoprenoid precursors and/or isoprenoids. These recombinant microorganisms comprise an enzyme capable of synthesizing acetoacetyl-CoA from malonyl-CoA and acetyl-CoA to produce acetoacetyl-CoA instead of an acetoacetyl-CoA thiolase enzyme capable of synthesizing acetoacetyl-CoA from two acetyl-CoA molecules.

Accordingly, in one aspect, the invention provides for a recombinant microorganism capable of producing isoprene comprising one or more nucleic acids encoding a polypeptide capable of synthesizing acetoacetyl-CoA from malonyl-CoA and acetyl-CoA and one or more nucleic acids encoding: (a) an isoprene synthase polypeptide, wherein the isoprene synthase polypeptide is encoded by a heterologous nucleic acid; and (b) one or more mevalonate (MVA) pathway polypeptides, wherein culturing of said recombinant microorganism in a suitable media provides for the production of said polypeptides and synthesis of isoprene. In one aspect, the one or more nucleic acids encoding a polypeptide capable of synthesizing acetoacetyl-CoA from malonyl-CoA and acetyl-CoA is an acetoacetyl-CoA synthase gene. In another aspect, the acetoacetyl-CoA synthase gene is a gene from an actinomycete. In another aspect, the acetoacetyl-CoA synthase gene is from the genus Streptomyces. In another aspect, the acetoacetyl-CoA synthase gene encodes a protein having the amino acid sequence of:

(SEQ ID NO: 1)
MTDVRFRIIGTGAYVPERIVSNDEVGAPAGVDDDWITRKTGIRQ
RRWAADDQATSDLATAAGRAALKAAGITPEQLTVIAVATSTPDRPQPPTA
AYVQHHLGATGTAAFDVNAVCSGTVFALSSVAGTLVYRGGYALVIGADLY
SRILNPADRKTVVLFGDGAGAMVLGPTSTGTGPIVRRVALHTFGGLTDLI
RVPAGGSRQPLDTDGLDAGLQYFAMDGREVRRFVTEHLPQLIKGFLHEAG
VDAADISHFVPHQANGVMLDEVFGELHLPRATMHRTVETYGNTGAASIPI
TMDAAVRAGSFRPGELVLLAGFGGGMAASFALIEW.

In another aspect, the acetoacetyl-CoA synthase gene encodes a protein having an amino acid sequence with an 80% or more identity to the amino acid sequence of SEQ ID NO: 1 and having a function of synthesizing acetoacetyl-CoA from malonyl-CoA and acetyl-CoA.

In any of the aspects herein, the isoprene synthase polypeptide is a plant isoprene synthase polypeptide. In any of the aspects herein, the isoprene synthase polypeptide is a polypeptide from Pueraria or Populus or a hybrid, Populus alba×Populus tremula. In any of the aspects herein, the isoprene synthase polypeptide is selected from the group consisting of Pueraria montana or Pueraria lobata, Populus tremuloides, Populus alba, Populus nigra, and Populus trichocarpa. In another aspect, the plant isoprene synthase polypeptide is a kudzu isoprene synthase polypeptide.

In any of the aspects herein, the one or more nucleic acids encoding one or more MVA pathway polypeptides is a heterologous nucleic acid. In any of the aspects herein, the one or more nucleic acids encoding more MVA pathway polypeptides is a copy of an endogenous nucleic acid. In any of the aspects herein, one or more MVA pathway polypeptides is selected from (a) an enzyme that condenses acetoacetyl-CoA with acetyl-CoA to form HMG-CoA (e.g., HMG synthase); (b) an enzyme that converts HMG-CoA to mevalonate; (c) an enzyme that phosphorylates mevalonate to mevalonate 5-phosphate; (d) an enzyme that converts mevalonate 5-phosphate to mevalonate 5-pyrophosphate; and (e) an enzyme that converts mevalonate 5-pyrophosphate to isopentenyl pyrophosphate.

In any of the aspects herein, the enzyme that phosphorylates mevalonate to mevalonate 5-phosphate can be selected from the group consisting of M. mazei mevalonate kinase, Lactobacillus mevalonate kinase polypeptide, Lactobacillus sakei mevalonate kinase polypeptide, yeast mevalonate kinase polypeptide, Saccharomyces cerevisiae mevalonate kinase polypeptide, Streptococcus mevalonate kinase polypeptide, Streptococcus pneumoniae mevalonate kinase polypeptide, and Streptomyces mevalonate kinase polypeptide, or Streptomyces CL190 mevalonate kinase polypeptide. In any of the aspects herein, the enzyme that phosphorylates mevalonate to mevalonate 5-phosphate is M. mazei mevalonate kinase.

In any of the aspects herein, the recombinant microorganism can further comprise one or more nucleic acids encoding one or more 1-deoxy-D-xylulose-5-phosphate (DXP) pathway polypeptides. In one aspect, one or more nucleic acids that encode for one or more DXP pathway polypeptides is a heterologous nucleic acid. In another aspect, one or more nucleic acids encoding one or more DXP pathway polypeptides is a copy of an endogenous nucleic acid. In another aspect, the one or more DXP pathway polypeptides is selected from (a) 1-deoxy-D-xylulose-5-phosphate synthase (DXS), (b) 1-deoxy-D-xylulose-5-phosphate reductoisomerase (DXR), (c) 4-diphosphocytidyl-2C-methyl-D-erythritol synthase (MCT), (d) 4-diphosphocytidyl-2-C-methyl-D-erythritol kinase (CMK), (e) 2C-methyl-D-erythritol 2,4-cyclodiphosphate synthase (MCS), (f) 1-hydroxy-2-methyl-2-(E)-butenyl 4-diphosphate synthase (HDS), and (g) 1-hydroxy-2-methyl-2-(E)-butenyl 4-diphosphate reductase (HDR). In another aspect, the DXP pathway polypeptide is DXS.

In any of the aspects herein, the one or more heterologous nucleic acids is placed under an inducible promoter or a constitutive promoter. In any of the aspects herein, the one or more heterologous nucleic acids is cloned into one or more multicopy plasmids. In any of the aspects herein, the one or more heterologous nucleic acids is integrated into a chromosome of the cells.

In any of the aspects herein, the microorganism is a bacterial, algal, fungal, yeast, or cyanobacterial cell. In one aspect, the microorganism is a bacterial cell. In another aspect, the bacterial cell is a gram-positive bacterial cell or gram-negative bacterial cell. In another aspect, the bacterial cell is selected from the group consisting of Escherichia sp. (e.g., E. coli), L. acidophilus, P. citrea, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, Corynebacterium spp. (e.g., C. glutamicum), S. degradans 2-40, Alginovibrio aqualiticus, Alteromonas sr. strain KLIA, Asteromyces cruciatus, Beneckea pelagia, Corynebacterium spp., Enterobacter cloacae, Halmonas marina, Klebsiella pneumonia, Photobacterium spp. (ATCC 433367), Pseudoalteromonas elyakovii, Pseudomonas sp. (e.g., Pseudomonas alginovora, Pseudomonas aeruginosa, Pseudomonas maltophilia, Pseudomonas putida), Vibrio alginolyticus, Vibrio halioticol, and Vibrio harveyi, S. albus, S. lividans, S. coelicolor, S. griseus, and P. alcaligenes cells. In another aspect, the bacterial cell is an E. coli cell. In another aspect, the bacterial cell is an L. acidophilus cell. In another aspect, the microorganism is an algal cell. In another aspect, the algal cell is selected from the group consisting of green algae, red algae, glaucophytes, chlorarachniophytes, euglenids, chromista, or dinoflagellates. In another aspect, the microorganism is a fungal cell. In another aspect, the fungal cell is a filamentous fungi. In another aspect, the microorganism is a yeast cell. In another aspect, the yeast cell is selected from the group consisting of Saccharomyces sp., Schizosaccharomyces sp., Pichia sp., or Candida sp. In another aspect, the yeast cell is a Saccharomyces cerevisiae cell.

In another aspect, the invention provides for a recombinant microorganism capable of producing an isoprenoid comprising one or more nucleic acids encoding a polypeptide capable of synthesizing acetoacetyl-CoA from malonyl-CoA and acetyl-CoA and one or more nucleic acids encoding: (a) one or more nucleic acids encoding a polyprenyl pyrophosphate synthase; and (b) one or more nucleic acids encoding one or more mevalonate (MVA) pathway polypeptides, wherein culturing of said recombinant microorganism in a suitable media provides for production of said polypeptides and synthesis of one or more isoprenoid(s). In one aspect, the one or more nucleic acids encoding one or more MVA pathway polypeptides of (b) is a heterologous nucleic acid. In any of the aspects herein, the one or more MVA pathway polypeptides is selected from the group consisting of (a) an enzyme that condenses acetoacetyl-CoA-CoA with acetyl-CoA to form HMG-Co-A; (b) an enzyme that converts HMG-CoA to mevalonate; (c) an enzyme that phosphorylates mevalonate to mevalonate 5-phosphate; (d) an enzyme that converts mevalonate 5-phosphate to mevalonate 5-pyrophosphate; and (e) an enzyme that converts mevalonate 5-pyrophosphate to isopentenyl pyrophosphate.

In any of the aspects herein, the enzyme that phosphorylates mevalonate to mevalonate 5-phosphate is selected from the group consisting of M. mazei mevalonate kinase, Lactobacillus mevalonate kinase polypeptide, Lactobacillus sakei mevalonate kinase polypeptide, yeast mevalonate kinase polypeptide, Saccharomyces cerevisiae mevalonate kinase polypeptide, Streptococcus mevalonate kinase polypeptide, Streptococcus pneumoniae mevalonate kinase polypeptide, and Streptomyces mevalonate kinase polypeptide, Streptomyces CL190 mevalonate kinase polypeptide. In one aspect, the enzyme that phosphorylates mevalonate to mevalonate 5-phosphate is M. mazei mevalonate kinase.

In any of the aspects herein, the one or more heterologous nucleic acids is placed under an inducible promoter or a constitutive promoter. In any of aspects herein, the one or more heterologous nucleic acids is cloned into one or more multicopy plasmids. In any of aspects herein, the one or more heterologous nucleic acids is integrated into a chromosome of the cells.

In one aspect, the microorganism is a bacterial, algal, fungal, yeast, or cyanobacterial cell. In one aspect, the microorganism is a bacterial cell. In another aspect, the bacterial cell is a gram-positive bacterial cell or gram-negative bacterial cell. In another aspect, the bacterial cell is selected from the group consisting of Escherichia sp. (e.g., E. coli), L. acidophilus, P. citrea, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, Corynebacterium spp. (e.g., C. glutamicum), S. degradans 2-40, Alginovibrio aqualiticus, Alteromonas sr. strain KLIA, Asteromyces cruciatus, Beneckea pelagia, Corynebacterium spp., Enterobacter cloacae, Halmonas marina, Klebsiella pneumonia, Photobacterium spp. (ATCC 433367), Pseudoalteromonas elyakovii, Pseudomonas sp. (e.g., Pseudomonas alginovora, Pseudomonas aeruginosa, Pseudomonas maltophilia, Pseudomonas putida), Vibrio alginolyticus, Vibrio halioticol, and Vibrio harveyi, S. albus, S. lividans, S. coelicolor, S. griseus, and P. alcaligenes cells. In another aspect, the bacterial cell is an E. coli cell. In another aspect, the bacterial cell is an L. acidophilus cell. In another aspect, the microorganism is an algal cell. In another aspect, the algal cell is selected from the group consisting of green algae, red algae, glaucophytes, chlorarachniophytes, euglenids, chromista, or dinoflagellates. In another aspect, the microorganism is a fungal cell. In another aspect, the fungal cell is a filamentous fungi. In another aspect, the microorganism is a yeast cell. In another aspect, the yeast cell is selected from the group consisting of Saccharomyces sp., Schizosaccharomyces sp., Pichia sp., or Candida sp. In another aspect, the yeast cell is a Saccharomyces cerevisiae cell.

In any of the aspects herein, the isoprenoid is selected from group consisting of monoterpenes, diterpenes, triterpenes, tetraterpenes, sequiterpene, and polyterpene. In one aspect, the isoprenoid is a sesquiterpene. In another aspect, the isoprenoid is selected from the group consisting of abietadiene, amorphadiene, carene, farnesene, α-farnesene, β-farnesene, farnesol, geraniol, geranylgeraniol, linalool, limonene, myrcene, nerolidol, ocimene, patchoulol, β-pinene, sabinene, γ-terpinene, terpindene and valencene.

In another aspect, the invention provides for methods of producing isoprene, the method comprising: (a) culturing a recombinant microorganism comprising one or more nucleic acids encoding (i) a polypeptide capable of synthesizing acetoacetyl-CoA from malonyl-CoA and acetyl-CoA and one or more nucleic acids encoding: (ii) an isoprene synthase polypeptide, wherein the isoprene synthase polypeptide is encoded by a heterologous nucleic acid; and (iii) one or more mevalonate (MVA) pathway polypeptides, and (b) producing isoprene. In one aspect, the method further comprises recovering the isoprene produced by the recombinant microorganism.

In another aspect, the or more nucleic acids encoding a polypeptide capable of synthesizing acetoacetyl Co-A from malonyl Co-A and acetyl-CoA is an acetoacetyl-CoA synthase gene. In another aspect, the isoprene synthase polypeptide is a plant isoprene synthase polypeptide. In another aspect, the one or more MVA pathway polypeptides is selected from the group consisting of (a) an enzyme that condenses acetoacetyl-CoA with acetyl-CoA to form HMG-Co-A; (b) an enzyme that converts HMG-CoA to mevalonate; (c) an enzyme that phosphorylates mevalonate to mevalonate 5-phosphate; (d) an enzyme that converts mevalonate 5-phosphate to mevalonate 5-pyrophosphate; and (e) an enzyme that converts mevalonate 5-pyrophosphate to isopentenyl pyrophosphate. In another aspect, the recombinant microorganism further comprises one or more nucleic acids encoding one or more 1-deoxy-D-xylulose-5-phosphate (DXP) pathway polypeptides.

In another aspect, the microorganism is a bacterial, algal, fungal or yeast cell. In another aspect, the microorganism is a bacterial cell. In another aspect, the bacterial cell is a gram-positive bacterial cell or gram-negative bacterial cell. In another aspect, the bacterial cell is an E. coli cell. In another aspect, the bacterial cell is an L. acidophilus cell. In another aspect, the microorganism is a yeast cell. In another aspect, the yeast cell is a Saccharomyces cerevisiae cell.

In another aspect, provided herein is a method of producing an isoprenoid, the method comprising: culturing a recombinant microorganism comprising one or more nucleic acids encoding (i) a polypeptide capable of synthesizing acetoacetyl-CoA from malonyl-CoA and acetyl-CoA and one or more nucleic acids encoding: (ii) a polyprenyl pyrophosphate synthase polypeptide, wherein the polyprenyl pyrophosphate synthase polypeptide is encoded by a heterologous nucleic acid; and (iii) one or more mevalonate (MVA) pathway polypeptides, and producing said isoprenoid.

### BRIEF DESCRIPTION OF THE DRAWINGS

FIG. 1 is a plasmid map of Strep CL190 Upper.

FIG. 2 is a plasmid map of pMCM1187.

FIG. 3 is a plasmid map of pCL-Ptrc-mvaR-mvaS-nphT7 isolated from strains MCM1320 and MCM1321.

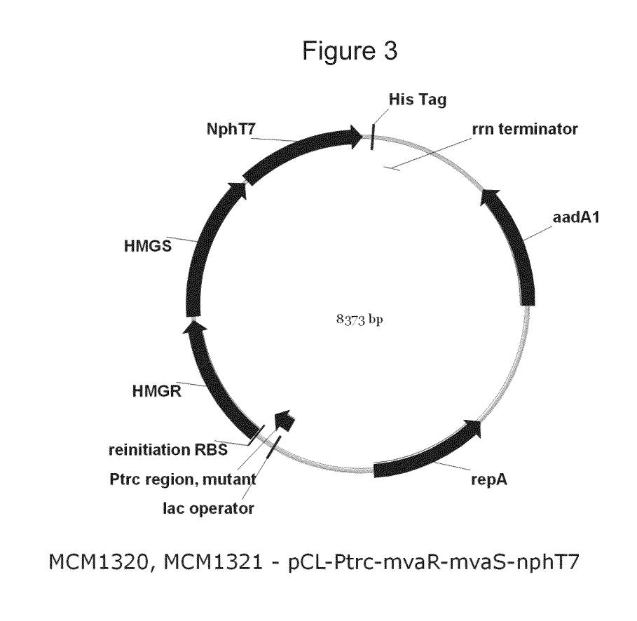

FIG. 4 is a graph showing the levels of isoprene produced by strains engineered to encode Acetoacetyl-CoA (NphT7). Isoprene levels were detected using a Gas Chromatography-Flame Ionization Detector. Strains MCM1684 and MCM1685, which produce MVA via Acetoacetyl-CoA, generated significantly higher levels of isoprene as compared to the MCM1686 strain that produces isoprene via the DXP pathway.

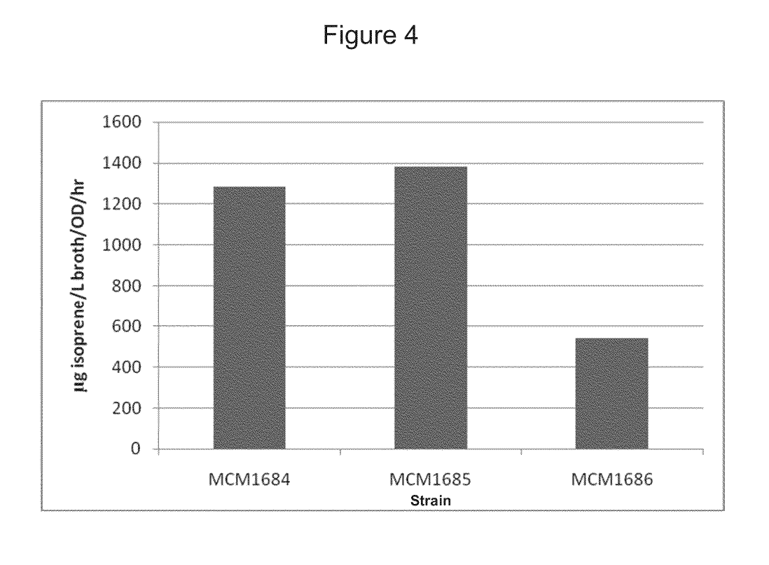

FIG. 5 is a vector map of construct pMCM1221.

FIG. 6 is a vector map of construct nphT7 with S suis HMGRS/pCL. The genes encoding the upper MVA pathway enzymes are highlighted in the figure, as well as the IPTG-inducible Trc promoter governing expression of the 3 gene operon. The HMG-CoA Reductase (HMGR) and the HMG-CoA Synthase (HMGS) enzymes are encoded by genes derived from Streptococcus suis and the NphT7 Acetoacetyl-CoA Synthase is encoded by the nphT7 gene derived from Streptomyces sp. strain CL190. The spectinomycin resistance gene (aadA1) and the gene encoding the RepA protein required for plasmid replication (repA) common to the pCL1920 vector backbone are included in the construct, but are not shown in the figure.

FIG. 7 is a graph depicting the specific productivity of isoprene (ug/L OD hr. units), represented by the black and gray bars, and the optical density (OD 600 nm), represented as a black diamond, for each of the cultures tested. Isoprene data for the strains harboring the upper MVA pathway enzymes consisting of the S. suis HMGR and HMGS together with NphT7 Acetoacetyl-CoA Synthase are depicted by the black bars (labeled NphT7 a-c in the graph representing REM C8_{—}25, REM C9_{—}25, and REM D1_{—}25 respectively); isoprene data for the strains harboring the upper MVA pathway enzymes encoded by nphT5, nphT6, and nphT7 genes derived from Streptomyces sp. strain CL190 are depicted by gray bars (labeled MCM1684 and MCM1685); isoprene data for the control strain which lacks an exogenous upper MVA pathway system is also shown in gray (labeled IspS alone). Isoprene specific productivity is represented on the left y-axis. The OD measurements were taken 3.5 hours post IPTG-induction of relevant gene expression and are represented on the right y-axis.

FIG. 8 is a graph showing NADP+/time/OD from a catalytic activity assay of coupled NphT7, HMG-CoA synthase, and HMG-CoA Reductase. The strains nphT7 test-strain 1-3 correspond to REM C8_{—}25, REM C9_{—}25, and REM D1_{—}25 respectively. The Control-Parental-IspS alone strain is REM F3_{—}25. These results are consistent with NphT7 activity dependence on the presence of both acetyl-CoA and malonyl-CoA.

FIG. 9 is a graph showing Isoprene/time/OD from catalytic assays using strains nphT7 test-strain 1-3, which correspond to REM C8_{—}25, REM C9_{—}25, and REM D1_{—}25 respectively. The Control-Parental-IspS alone strain is REM F3_{—}25.

### DETAILED DESCRIPTION

The invention provides, inter alia, compositions and methods for the increased production of isoprene, isoprenoid precursor molecules, and/or isoprenoids in recombinant microorganisms that have been engineered to express an enzyme capable of synthesizing acetoacetyl-CoA from malonyl-CoA and acetyl-CoA as the first step in directing carbon flux towards the production of isoprene, isoprenoid precursor and/or isoprenoids.

### General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, “Molecular Cloning: A Laboratory Manual”, third edition (Sambrook et al., 2001); “Oligonucleotide Synthesis” (M. J. Gait, ed., 1984); “Animal Cell Culture: A practical approach”, third edition (J. R. Masters, ed., 2000); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994). Singleton et al., Dictionary of Microbiology and Molecular Biology 3rd revised ed., J. Wiley & Sons (New York, N.Y. 2006), and March's Advanced Organic Chemistry Reactions, Mechanisms and Structure 6th ed., John Wiley & Sons (New York, N.Y. 2007), provide one skilled in the art with a general guide to many of the terms used in the present application.

### DEFINITIONS

The term “isoprene” refers to 2-methyl-1,3-butadiene (CAS#78-79-5). It can be the direct and final volatile C5 hydrocarbon product from the elimination of pyrophosphate from 3,3-dimethylallyl diphosphate (DMAPP). It may not involve the linking or polymerization of IPP molecules to DMAPP molecules. The term “isoprene” is not generally intended to be limited to its method of production unless indicated otherwise herein.

As used herein, the term “polypeptides” includes polypeptides, proteins, peptides, fragments of polypeptides, and fusion polypeptides.

As used herein, an “isolated polypeptide” is not part of a library of polypeptides, such as a library of 2, 5, 10, 20, 50 or more different polypeptides and is separated from at least one component with which it occurs in nature. An isolated polypeptide can be obtained, for example, by expression of a recombinant nucleic acid encoding the polypeptide.

By “heterologous polypeptide” is meant a polypeptide encoded by a nucleic acid sequence derived from a different organism, species, or strain than the host cell. In some embodiments, a heterologous polypeptide is not identical to a wild-type polypeptide that is found in the same host cell in nature.

As used herein, a “nucleic acid” refers to two or more deoxyribonucleotides and/or ribonucleotides covalently joined together in either single or double-stranded form.

By “recombinant nucleic acid” is meant a nucleic acid of interest that is free of one or more nucleic acids (e.g., genes) which, in the genome occurring in nature of the organism from which the nucleic acid of interest is derived, flank the nucleic acid of interest. The term therefore includes, for example, a recombinant DNA which is incorporated into a vector, into an autonomously replicating plasmid or virus, or into the genomic DNA of a prokaryote or eukaryote, or which exists as a separate molecule (e.g., a cDNA, a genomic DNA fragment, or a cDNA fragment produced by PCR or restriction endonuclease digestion) independent of other sequences.

By “heterologous nucleic acid” is meant a nucleic acid sequence derived from a different organism, species or strain than the host cell. In some embodiments, the heterologous nucleic acid is not identical to a wild-type nucleic acid that is found in the same host cell in nature.

As used herein, an “expression control sequence” means a nucleic acid sequence that directs transcription of a nucleic acid of interest. An expression control sequence can be a promoter, such as a constitutive or an inducible promoter, or an enhancer. An expression control sequence can be “native” or heterologous. A native expression control sequence is derived from the same organism, species, or strain as the gene being expressed. A heterologous expression control sequence is derived from a different organism, species, or strain as the gene being expressed. An “inducible promoter” is a promoter that is active under environmental or developmental regulation.

By “operably linked” is meant a functional linkage between a nucleic acid expression control sequence (such as a promoter) and a second nucleic acid sequence, wherein the expression control sequence directs transcription of the nucleic acid corresponding to the second sequence.

As used herein, the terms “minimal medium” or “minimal media” refer to growth medium containing the minimum nutrients possible for cell growth, generally without the presence of amino acids. Minimal medium typically contains: (1) a carbon source for cell growth; (2) various salts, which can vary among host cell species and growing conditions; and (3) water. The carbon source can vary significantly, from simple sugars like glucose to more complex hydrolysates of other biomass, such as yeast extract, as discussed in more detail below. The salts generally provide essential elements such as magnesium, nitrogen, phosphorus, and sulfur to allow the cells to synthesize proteins and nucleic acids. Minimal medium can also be supplemented with selective agents, such as antibiotics, to select for the maintenance of certain plasmids and the like. For example, if a microorganism is resistant to a certain antibiotic, such as ampicillin or tetracycline, then that antibiotic can be added to the medium in order to prevent cells lacking the resistance from growing. Medium can be supplemented with other compounds as necessary to select for desired physiological or biochemical characteristics, such as particular amino acids and the like.

As used herein, the term “isoprenoid” refers to a large and diverse class of naturally-occurring class of organic compounds composed of two or more units of hydrocarbons, with each unit consisting of five carbon atoms arranged in a specific pattern. As used herein, “isoprene” is expressly excluded from the definition of “isoprenoid.”

As used herein, the term “terpenoid” refers to a large and diverse class of organic molecules derived from five-carbon isoprenoid units assembled and modified in a variety of ways and classified in groups based on the number of isoprenoid units used in group members. Hemiterpenoids have one isoprenoid unit. Monoterpenoids have two isoprenoid units. Sesquiterpenoids have three isoprenoid units. Diterpenoids have four isoprene units. Sesterterpenoids have five isoprenoid units. Triterpenoids have six isoprenoid units. Tetraterpenoids have eight isoprenoid units. Polyterpenoids have more than eight isoprenoid units.

As used herein, “isoprenoid precursor” refers to any molecule that is used by organisms in the biosynthesis of terpenoids or isoprenoids. Non-limiting examples of isoprenoid precursor molecules include, e.g., isopentenyl pyrophosphate (IPP) and dimethylallyl diphosphate (DMAPP).

As used herein, the term “mass yield” refers to the mass of the product produced by the host cells divided by the mass of the glucose consumed by the host cells multiplied by 100.

By “specific productivity,” it is meant the mass of the product produced by the host cell divided by the product of the time for production, the host cell density, and the volume of the culture.

By “titer,” it is meant the mass of the product produced by the host cells divided by the volume of the culture.

As used herein, the term “cell productivity index (CPI)” refers to the mass of the product produced by the host cells divided by the mass of the host cells produced in the culture.

Unless defined otherwise herein, all technical and scientific terms used herein have the same meaning as commonly understood by one of ordinary skill in the art to which this invention pertains.

As used herein, the singular terms “a,” “an,” and “the” include the plural reference unless the context clearly indicates otherwise.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Recombinant Microorganisms Capable of Production of Isoprene, Isoprenoid Precursors or Isoprenoids

The mevalonate-dependent biosynthetic pathway (MVA pathway) is a key metabolic pathway present in all higher eukaryotes and certain bacteria. In addition to being important for the production of molecules used in processes as diverse as protein prenylation, cell membrane maintenance, protein anchoring, and N-glycosylation, the mevalonate pathway provides a major source of the isoprenoid precursor molecules DMAPP and IPP, which serve as the basis for the biosynthesis of terpenes, terpenoids, isoprenoids, and isoprene.

As described herein, the upper portion of the MVA pathway utilizes acetyl Co-A and malonyl Co-A produced during cellular metabolism as the initial substrates for the production of mevalonate via the actions of polypeptides having acetoacetyl-CoA synthase, HMG-CoA reductase, and HMG-CoA synthase enzymatic activity. First, acetyl Co-A and malonyl Co-A are converted to acetoacetyl CoA via the action of an acetoacetyl-CoA synthase. Next, acetoacetyl CoA is converted to 3-hydroxy-3-methylglutaryl-CoA (HMG-CoA) by the enzymatic action of HMG-CoA synthase. This Co-A derivative is reduced to mevalonate by HMG-CoA reductase, which is the rate-limiting step of the mevalonate pathway of isoprenoid production. Mevalonate is then converted into mevalonate-5-phosphate via the action of mevalonate kinase which is subsequently transformed into mevalonate-5-pyrophosphate by the enzymatic activity of phosphomevalonate kinase. Finally, IPP is formed from mevalonate-5-pyrophosphate by the activity of the enzyme mevalonate-5-pyrophosphate decarboxylase.

Thus, the recombinant microorganisms of the present invention are recombinant microorganisms having the ability to produce isoprene, isoprenoid precursors or isoprenoids wherein the recombinant microorganisms comprise by a gene encoding an enzyme capable of synthesizing acetoacetyl-CoA from malonyl-CoA and acetyl-CoA (e.g., acetoacetyl-CoA synthase gene or nphT7) and a one or more of a group of genes involved in isoprene biosynthesis or isoprenoid biosynthesis that enables the synthesis of isoprene or isoprenoids from acetoacetyl-CoA in the host microorganism.

### Acetoacetyl-CoA Synthase Gene

The acetoacetyl-CoA synthase gene (aka nphT7) is a gene encoding an enzyme having the activity of synthesizing acetoacetyl-CoA from malonyl-CoA and acetyl-CoA and having minimal activity (e.g., no activity) of synthesizing acetoacetyl-CoA from two acetyl-CoA molecules. See, e.g., Okamura et al., PNAS Vol 107, No. 25, pp. 11265-11270 (2010), the contents of which are expressly incorporated herein for teaching about nphT7. An acetoacetyl-CoA synthase gene from an actinomycete of the genus Streptomyces CL190 strain was described in JP Patent Publication (Kokai) No. 2008-61506 A and US2010/0285549. Acetoacetyl-CoA synthase can also be referred to as acetyl CoA:malonyl CoA acyltransferase. A representative acetoacetyl-CoA synthase (or acetyl CoA:malonyl CoA acyltransferase) that can be used is Genbank AB540131.1.

In one embodiment, acetoacetyl-CoA synthase of the present invention synthesizes acetoacetyl-CoA from malonyl-CoA and acetyl-CoA via an irreversible reaction. The use of acetoacetyl-CoA synthase to generate acetyl-CoA provides an additional advantage in that this reaction is irreversible while acetoacetyl-CoA thiolase enzyme's action of synthesizing acetoacetyl-CoA from two acetyl-CoA molecules is reversible. Consequently, the use of acetoacetyl-CoA synthase to synthesize acetoacetyl-CoA from malonyl-CoA and acetyl-CoA can result in significant improvement in productivity for isoprene, isoprenoid precursors and/or isoprenoids, compared with using thiolase to generate the end same products.

Furthermore, the use of acetoacetyl-CoA synthase to produce isoprene, isoprenoid precursors and/or isoprenoids provides another advantage in that acetoacetyl-CoA synthase can convert malonyl CoA to acetyl CoA via decarboxylation of the malonyl CoA. Thus, the stores of starting substrate is not limited by the starting amounts of acetyl CoA. The synthesis of acetoacetyl-CoA by acetoacetyl-CoA synthase can still occur when the starting substrate is only malonyl-CoA. In one embodiment, the pool of starting malonyl-CoA is increased by using host strains that have more malonyl-CoA. Such increased pools can be naturally occurring or be engineered by molecular manipulation. See, for example Fowler, et. al, Applied and Environmental Microbiology, Vol. 75, No. 18, pp. 5831-5839 (2009), Zha et al., Metabolic Engineering, 11: 192-198 (2009), Xu et al., Metabolic Engineering, (2011)doi:10.1016/j.ymben.2011.06.008, Okamura et al., PNAS 107: 11265-11270 (2010), and US 2010/0285549, the contents of which are expressly incorporated herein by reference in their entirety.

In any of the aspects or embodiments described herein, an enzyme that has the ability to synthesize acetoacetyl-CoA from malonyl-CoA and acetyl-CoA can be used. Non-limiting examples of such an enzyme are described herein. In certain embodiments described herein, an acetoacetyl-CoA synthase gene derived from an actinomycete of the genus Streptomyces having the activity of synthesizing acetoacetyl-CoA from malonyl-CoA and acetyl-CoA can be used.

An example of such an acetoacetyl-CoA synthase gene is the gene encoding a protein having the amino acid sequence of SEQ ID NO: 1. Such a protein having the amino acid sequence of SEQ ID NO: 1 corresponds to an acetoacetyl-CoA synthase having activity of synthesizing acetoacetyl-CoA from malonyl-CoA and acetyl-CoA and having no activity of synthesizing acetoacetyl-CoA from two acetyl-CoA molecules.

In one embodiment, the gene encoding a protein having the amino acid sequence of SEQ ID NO: 1 can be obtained by a nucleic acid amplification method (e.g., PCR) with the use of genomic DNA obtained from an actinomycete of the Streptomyces sp. CL190 strain as a template and a pair of primers that can be designed with reference to JP Patent Publication (Kokai) No. 2008-61506 A.

As described herein, an acetoacetyl-CoA synthase gene for use in the present invention is not limited to a gene encoding a protein having the amino acid sequence of SEQ ID NO: 1 from an actinomycete of the Streptomyces sp. CL190 strain. Any gene encoding a protein having the ability to synthesize acetoacetyl-CoA from malonyl-CoA and acetyl-CoA and which does not synthesize acetoacetyl-CoA from two acetyl-CoA molecules can be used in the presently described methods. In certain embodiments, the acetoacetyl-CoA synthase gene can be a gene encoding a protein having an amino acid sequence with high similarity or substantially identical to the amino acid sequence of SEQ ID NO: 1 and having the function of synthesizing acetoacetyl-CoA from malonyl-CoA and acetyl-CoA. The expression “highly similar” or “substantially identical” refers to, for example, at least about 80% identity, at least about 85%, at least about 90%, at least about 91%, at least about 92%, at least about 93%, at least about 94%, at least about 95%, at least about 96%, at least about 97%, at least about 98%, and at least about 99% identity. As used above, the identity value corresponds to the percentage of identity between amino acid residues in a different amino acid sequence and the amino acid sequence of SEQ ID NO: 1, which is calculated by performing alignment of the amino acid sequence of SEQ ID NO: 1 and the different amino acid sequence with the use of a program for searching for a sequence similarity.

In other embodiments, the acetoacetyl-CoA synthase gene may be a gene encoding a protein having an amino acid sequence derived from the amino acid sequence of SEQ ID NO: 1 by substitution, deletion, addition, or insertion of 1 or more amino acid(s) and having the function of synthesizing acetoacetyl-CoA from malonyl-CoA and acetyl-CoA. Herein, the expression “more amino acids” refers to, for example, 2 to 30 amino acids, preferably 2 to 20 amino acids, more preferably 2 to 10 amino acids, and most preferably 2 to 5 amino acids.

In still other embodiments, the acetoacetyl-CoA synthase gene may consist of a polynucleotide capable of hybridizing to a portion or the entirety of a polynucleotide having a nucleotide sequence complementary to the nucleotide sequence encoding the amino acid sequence of SEQ ID NO: 1 under stringent conditions and capable of encoding a protein having the function of synthesizing acetoacetyl-CoA from malonyl-CoA and acetyl-CoA. Herein, hybridization under stringent conditions corresponds to maintenance of binding under conditions of washing at 60.degree. C. 2.times.SSC. Hybridization can be carried out by conventionally known methods such as the method described in J. Sambrook et al. Molecular Cloning, A Laboratory Manual, 3rd Ed., Cold Spring Harbor Laboratory (2001).

As described herein, a gene encoding an acetoacetyl-CoA synthase having an amino acid sequence that differs from the amino acid sequence of SEQ ID NO: 1 can be isolated from potentially any organism, for example, an actinomycete that is not obtained from the Streptomyces sp. CL190 strain. In addition, acetoacetyl-CoA synthase genes for use herein can be obtained by modifying a polynucleotide encoding the amino acid sequence of SEQ ID NO: 1 by a method known in the art. Mutagenesis of a nucleotide sequence can be carried out by a known method such as the Kunkel method or the gapped duplex method or by a method similar to either thereof. For instance, mutagenesis may be carried out with the use of a mutagenesis kit (e.g., product names; Mutant-K and Mutant-G (TAKARA Bio)) for site-specific mutagenesis, product name; an LA PCR in vitro Mutagenesis series kit (TAKARA Bio), and the like.

The activity of an acetoacetyl-CoA synthase having an amino acid sequence that differs from the amino acid sequence of SEQ ID NO: 1 can be evaluated as described below. Specifically, a gene encoding a protein to be evaluated is first introduced into a host cell such that the gene can be expressed therein, followed by purification of the protein by a technique such as chromatography. Malonyl-CoA and acetyl-CoA are added as substrates to a buffer containing the obtained protein to be evaluated, followed by, for example, incubation at a desired temperature (e.g., 10° C. to 60° C.). After the completion of reaction, the amount of substrate lost and/or the amount of product (acetoacetyl-CoA) produced are determined. Thus, it is possible to evaluate whether or not the protein being tested has the function of synthesizing acetoacetyl-CoA from malonyl-CoA and acetyl-CoA and to evaluate the degree of synthesis. In such case, it is possible to examine whether or not the protein has the activity of synthesizing acetoacetyl-CoA from two acetyl-CoA molecules by adding acetyl-CoA alone as a substrate to a buffer containing the obtained protein to be evaluated and determining the amount of substrate lost and/or the amount of product produced in a similar manner.

### MVA Pathway Nucleic Acids and Polypeptides

Exemplary MVA pathway polypeptides that can be used in conjunction with acetoacetyl-CoA synthase include, but are not limited to: 3-hydroxy-3-methylglutaryl-CoA synthase (HMG-CoA synthase) polypeptides (e.g., an enzyme encoded by mvaS), 3-hydroxy-3-methylglutaryl-CoA reductase (HMG-CoA reductase) polypeptides (e.g., enzyme encoded by mvaR or enzyme encoded by mvaE that has been modified to be thiolase-deficient but still retains its reductase activity), mevalonate kinase (MVK) polypeptides, phosphomevalonate kinase (PMK) polypeptides, diphosphomevalonte decarboxylase (MVD) polypeptides, phosphomevalonate decarboxylase (PMDC) polypeptides, isopentenyl phosphate kinase (IPK) polypeptides, IPP isomerase polypeptides, IDI polypeptides, and polypeptides (e.g., fusion polypeptides) having an activity of two or more MVA pathway polypeptides. In particular, MVA pathway polypeptides include polypeptides, fragments of polypeptides, peptides, and fusions polypeptides that have at least one activity of an MVA pathway polypeptide. Exemplary MVA pathway nucleic acids include nucleic acids that encode a polypeptide, fragment of a polypeptide, peptide, or fusion polypeptide that has at least one activity of an MVA pathway polypeptide. Exemplary MVA pathway polypeptides and nucleic acids include naturally-occurring polypeptides and nucleic acids from any of the source organisms described herein. In addition, variants of MVA pathway polypeptide that confer the result of better isoprene production can also be used as well.

Non-limiting examples of MVA pathway polypeptides which can be used are described in International Patent Application Publication No. WO2009/076676; WO2010/003007 and WO2010/148150, the contents of which are expressing incorporated by reference herein.

### Exemplary 3-Hydroxy-3-Methylglutaryl-CoA Reductase (HMG-CoA Reductase) Polypeptides and Nucleic Acids

Enzymes that catalyze the reaction that convert HMG-CoA to mevalonate polypeptides can be used, for example, 3-hydroxy-3-methylglutaryl-CoA reductase (HMG-CoA reductase). Another example is an enzyme that is coded by mvaE that has been modified to be thiolase-deficient but still retains its reductase activity. It has been reported that mvaE gene encodes a polypeptide that possesses both thiolase and HMG-CoA reductase activities. The thiolase activity of the polypeptide encoded by the mvaE gene converts acetyl Co-A to acetoacetyl CoA whereas the HMG-CoA reductase enzymatic activity of the polypeptide converts 3-hydroxy-3-methylglutaryl-CoA to mevalonate. Exemplary mvaE polypeptides and nucleic acids that can be used for this invention include naturally-occurring or modified polypeptides and nucleic acids from any of the source organisms described herein that do not have thiolase activity but have HMG-CoA reductase activity.

Modified mvaE polypeptides include those in which one or more amino acid residues have undergone an amino acid substitution while retaining HMG-CoA reductase activity while having minimal or no thiolase activity. The amino acid substitutions can be conservative or non-conservative and such substituted amino acid residues can or can not be one encoded by the genetic code. The standard twenty amino acid “alphabet” has been divided into chemical families based on similarity of their side chains. Those families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). A “conservative amino acid substitution” is one in which the amino acid residue is replaced with an amino acid residue having a chemically similar side chain (i.e., replacing an amino acid having a basic side chain with another amino acid having a basic side chain). A “non-conservative amino acid substitution” is one in which the amino acid residue is replaced with an amino acid residue having a chemically different side chain (i.e., replacing an amino acid having a basic side chain with another amino acid having an aromatic side chain).

Amino acid substitutions in the mvaE polypeptide can be introduced to improve the functionality of the molecule. For example, amino acid substitutions improve its ability to convert 3-hydroxy-3-methylglutaryl-CoA to mevalonate can be introduced into the thiolase-deficient mvaE polypeptide. In some aspects, the thiolase-deficient mvaE polypeptides contain one or more conservative amino acid substitutions.

In one aspect, thiolase-deficient mvaE proteins that are not degraded or less prone to degradation can be used for the production of mevalonate, isoprene, isoprenoid precursors, and/or isoprenoids. Examples of gene products of mvaEs that are not degraded or less prone to degradation which can be used include, but are not limited to, those from the organisms E. faecium, E. gallinarum, E. casseliflavus, E. faecalis, and L. grayi. One of skill in the art can express mvaE protein in E. coli BL21 (DE3) and look for absence of fragments by any standard molecular biology techniques. For example, absence of fragments can be identified on Safestain stained SDS-PAGE gels following His-tag mediated purification or when expressed in mevalonate, isoprene or isoprenoid producing E. coli BL21 using the methods of detection described herein.

Standard methods, such as those described in Hedl et al., (J Bacteriol. 2002, April; 184(8): 2116-2122) can be used to determine whether a polypeptide has thiolase-deficient, HMG CoA reductase-proficient mvaE activity, by measuring the absence of acetoacetyl-CoA thiolase and/or the presence of HMG-CoA reductase activity. In an exemplary assay, acetoacetyl-CoA thiolase activity is measured by spectrophotometer to monitor the change in absorbance at 302 nm that accompanies the formation or thiolysis of acetoacetyl-CoA. Standard assay conditions for each reaction to determine synthesis of acetoacetyl-CoA, are 1 mM acetyl-CoA, 10 mM MgCl₂, 50 mM Tris, pH 10.5 and the reaction is initiated by addition of enzyme. Assays can employ a final volume of 200 μl. For the assay, 1 enzyme unit (eu) represents the synthesis or thiolysis in 1 min of 1 μmol of acetoacetyl-CoA. In another exemplary assay, of HMG-CoA reductase activity can be monitored by spectrophotometer by the appearance or disappearance of NADP(H) at 340 nm. Standard assay conditions for each reaction measured to show reductive deacylation of HMG-CoA to mevalonate are 0.4 mM NADPH, 1.0 mM (R,S)-HMG-CoA, 100 mM KCl, and 100 mM K ₓPO₄, pH 6.5. Assays employ a final volume of 200 μl. Reactions are initiated by adding the enzyme. For the assay, 1 eu represents the turnover, in 1 min, of 1 μmol of NADP(H). This corresponds to the turnover of 0.5 μmol of HMG-CoA or mevalonate.

Alternatively, production of mevalonate in host cells can be measured by, without limitation, gas chromatography (see U.S. Patent Application Publication No.: US 2005/0287655 A1) or HPLC (See U.S. patent application Ser. No. 12/978,324). As an exemplary assay, cultures can be inoculated in shake tubes containing LB broth supplemented with one or more antibiotics and incubated for 14 h at 34° C. at 250 rpm. Next, cultures can be diluted into well plates containing TM3 media supplemented with 1% Glucose, 0.1% yeast extract, and 200 μM IPTG to final OD of 0.2. The plate are then sealed with a Breath Easier membrane (Diversified Biotech) and incubated at 34° C. in a shaker/incubator at 600 rpm for 24 hours. 1 mL of each culture is then centrifuged at 3,000×g for 5 min. Supernatant is then added to 20% sulfuric acid and incubated on ice for 5 min. The mixture is then centrifuged for 5 min at 3000×g and the supernatant was collected for HPLC analysis. The concentration of mevalonate in samples is determined by comparison to a standard curve of mevalonate (Sigma). The glucose concentration can additionally be measured by performing a glucose oxidase assay according to any method known in the art. Using HPLC, levels of mevalonate can be quantified by comparing the refractive index response of each sample versus a calibration curve generated by running various mevalonate containing solutions of known concentration.

HMG-CoA reductase can be expressed in a host cell on a multicopy plasmid. The plasmid can be a high copy plasmid, a low copy plasmid, or a medium copy plasmid. Alternatively, HMG-CoA reductase can be integrated into the host cell's chromosome. For both heterologous expression of HMG-CoA reductase on a plasmid or as an integrated part of the host cell's chromosome, expression of the nucleic acid can be driven by either an inducible promoter or a constitutively expressing promoter. The promoter can be a strong driver of expression, it can be a weak driver of expression, or it can be a medium driver of expression of the HMG-CoA reductase.

Exemplary HMG-CoA Synthase Polypeptides and Nucleic Acids

Enzymes that catalyze the conversion of acetoacetyl-CoA to HMG-CoA (e.g., HMG-CoA synthase or HMGS) can be used. In one embodiment, the polypeptide encoded by mvaS gene can be used. The mvaS gene encodes a polypeptide that possesses HMG-CoA synthase activity. This polypeptide can convert acetoacetyl CoA to 3-hydroxy-3-methylglutaryl-CoA (HMG-CoA). Exemplary mvaS polypeptides and nucleic acids include naturally-occurring polypeptides and nucleic acids from any of the source organisms described herein as well as mutant polypeptides and nucleic acids derived from any of the source organisms described herein that have at least one activity of a mvaS polypeptide.

Mutant mvaS polypeptides include those in which one or more amino acid residues have undergone an amino acid substitution while retaining mvaS polypeptide activity (i.e., the ability to convert acetoacetyl CoA to 3-hydroxy-3-methylglutaryl-CoA). Amino acid substitutions in the mvaS polypeptide can be introduced to improve the functionality of the molecule. For example, amino acid substitutions that increase the binding affinity of the mvaS polypeptide for its substrate, or that improve its ability to convert acetoacetyl CoA to 3-hydroxy-3-methylglutaryl-CoA can be introduced into the mvaS polypeptide. In some aspects, the mutant mvaS polypeptides contain one or more conservative amino acid substitutions.

Standard methods, such as those described in Quant et al. (Biochem J., 1989, 262:159-164), can be used to determine whether a polypeptide has mvaS activity, by measuring HMG-CoA synthase activity. In an exemplary assay, HMG-CoA synthase activity can be assayed by spectrophotometrically measuring the disappearance of the enol form of acetoacetyl-CoA by monitoring the change of absorbance at 303 nm. A standard 1 ml assay system containing 50 mm-Tris/HCl, pH 8.0, 10 mM-MgCl2 and 0.2 mM-dithiothreitol at 30° C.; 5 mM-acetyl phosphate, 10,M-acetoacetyl-CoA and 5 ul samples of extracts can be added, followed by simultaneous addition of acetyl-CoA (100 uM) and 10 units of PTA. HMG-CoA synthase activity is then measured as the difference in the rate before and after acetyl-CoA addition. The absorption coefficient of acetoacetyl-CoA under the conditions used (pH 8.0, 10 mM-MgCl2), is 12.2×10³ M⁻¹ cm⁻¹. By definition, 1 unit of enzyme activity causes 1 umol of acetoacetyl-CoA to be transformed per minute.

Alternatively, production of mevalonate in host cells can be measured by, without limitation, gas chromatography (see U.S. Patent Application Publication No.: US 2005/0287655 A1) or HPLC (See U.S. patent application Ser. No. 12/978,324). As an exemplary assay, cultures can be inoculated in shake tubes containing LB broth supplemented with one or more antibiotics and incubated for 14 h at 34° C. at 250 rpm. Next, cultures can be diluted into well plates containing TM3 media supplemented with 1% Glucose, 0.1% yeast extract, and 200 μM IPTG to final OD of 0.2. The plate are then sealed with a Breath Easier membrane (Diversified Biotech) and incubated at 34° C. in a shaker/incubator at 600 rpm for 24 hours. 1 mL of each culture is then centrifuged at 3,000×g for 5 min. Supernatant is then added to 20% sulfuric acid and incubated on ice for 5 min. The mixture is then centrifuged for 5 min at 3000×g and the supernatant was collected for HPLC analysis. The concentration of mevalonate in samples is determined by comparison to a standard curve of mevalonate (Sigma). The glucose concentration can additionally be measured by performing a glucose oxidase assay according to any method known in the art. Using HPLC, levels of mevalonate can be quantified by comparing the refractive index response of each sample versus a calibration curve generated by running various mevalonate containing solutions of known concentration.

The mvaS nucleic acid can be expressed in a host cell on a multicopy plasmid. The plasmid can be a high copy plasmid, a low copy plasmid, or a medium copy plasmid. Alternatively, the mvaS nucleic acid can be integrated into the host cell's chromosome. For both heterologous expression of an mvaS nucleic acid on a plasmid or as an integrated part of the host cell's chromosome, expression of the nucleic acid can be driven by either an inducible promoter or a constitutively expressing promoter. The promoter can be a strong driver of expression, it can be a weak driver of expression, or it can be a medium driver of expression of the mvaS nucleic acid.

Nucleic Acids Encoding Polypeptides of the Lower MVA Pathway

In some aspects of the invention, the cells described in any of the compositions or methods described herein further comprise one or more nucleic acids encoding a lower mevalonate (MVA) pathway polypeptide(s). In some aspects, the lower MVA pathway polypeptide is an endogenous polypeptide. In some aspects, the endogenous nucleic acid encoding a lower MVA pathway polypeptide is operably linked to a constitutive promoter. In some aspects, the endogenous nucleic acid encoding a lower MVA pathway polypeptide is operably linked to an inducible promoter. In some aspects, the endogenous nucleic acid encoding a lower MVA pathway polypeptide is operably linked to a strong promoter. In a particular aspect, the cells are engineered to over-express the endogenous lower MVA pathway polypeptide relative to wild-type cells. In some aspects, the endogenous nucleic acid encoding a lower MVA pathway polypeptide is operably linked to a weak promoter.

The lower mevalonate biosynthetic pathway comprises mevalonate kinase (MVK), phosphomevalonate kinase (PMK), and diphosphomevalonte decarboxylase (MVD). In some aspects, the lower MVA pathway can further comprise isopentenyl diphosphate isomerase (IDI). Cells provided herein can comprise at least one nucleic acid encoding isoprene synthase, one or more upper MVA pathway polypeptides, and/or one or more lower MVA pathway polypeptides. Polypeptides of the lower MVA pathway can be any enzyme (a) that phosphorylates mevalonate to mevalonate 5-phosphate; (b) that converts mevalonate 5-phosphate to mevalonate 5-pyrophosphate; and (c) that converts mevalonate 5-pyrophosphate to isopentenyl pyrophosphate. More particularly, the enzyme that phosphorylates mevalonate to mevalonate 5-phosphate can be from the group consisting of M. mazei mevalonate kinase, Lactobacillus mevalonate kinase polypeptide, Lactobacillus sakei mevalonate kinase polypeptide, yeast mevalonate kinase polypeptide, Saccharomyces cerevisiae mevalonate kinase polypeptide, Streptococcus mevalonate kinase polypeptide, Streptococcus pneumoniae mevalonate kinase polypeptide, Streptomyces mevalonate kinase polypeptide, Streptomyces CL190 mevalonate kinase polypeptide, and M. Burtonii mevalonate kinase polypeptide. In another aspect, the enzyme that phosphorylates mevalonate to mevalonate 5-phosphate is M. mazei mevalonate kinase.

In some aspects, the lower MVA pathway polypeptide is a heterologous polypeptide. In some aspects, the cells comprise more than one copy of a heterologous nucleic acid encoding a lower MVA pathway polypeptide. In some aspects, the heterologous nucleic acid encoding a lower MVA pathway polypeptide is operably linked to a constitutive promoter. In some aspects, the heterologous nucleic acid encoding a lower MVA pathway polypeptide is operably linked to an inducible promoter. In some aspects, the heterologous nucleic acid encoding a lower MVA pathway polypeptide is operably linked to a strong promoter. In some aspects, the heterologous nucleic acid encoding a lower MVA pathway polypeptide is operably linked to a weak promoter. In some aspects, the heterologous lower MVA pathway polypeptide is a polypeptide from Saccharomyces cerevisiae, Enterococcus faecalis, or Methanosarcina mazei.

The nucleic acids encoding a lower MVA pathway polypeptide(s) can be integrated into a genome of the cells or can be stably expressed in the cells. The nucleic acids encoding a lower MVA pathway polypeptide(s) can additionally be on a vector.

Exemplary lower MVA pathway polypeptides are also provided below: (i) mevalonate kinase (MVK); (ii) phosphomevalonate kinase (PMK); (iii) diphosphomevalonate decarboxylase (MVD); and (iv) isopentenyl diphosphate isomerase (IDI). In particular, the lower MVK polypeptide can be from the genus Methanosarcina and, more specifically, the lower MVK polypeptide can be from Methanosarcina mazei. Additional examples of lower MVA pathway polypeptides can be found in U.S. Patent Application Publication 2010/0086978 the contents of which are expressly incorporated herein by reference in their entirety with respect to lower MVK pathway polypeptides and lower MVK pathway polypeptide variants.

Any one of the cells described herein can comprise IDI nucleic acid(s) (e.g., endogenous or heterologous nucleic acid(s) encoding IDI). Isopentenyl diphosphate isomerase polypeptides (isopentenyl-diphosphate delta-isomerase or IDI) catalyzes the interconversion of isopentenyl diphosphate (IPP) and dimethylallyl diphosphate (DMAPP) (e.g., converting IPP into DMAPP and/or converting DMAPP into IPP). Exemplary IDI polypeptides include polypeptides, fragments of polypeptides, peptides, and fusions polypeptides that have at least one activity of an IDI polypeptide. Standard methods (such as those described herein) can be used to determine whether a polypeptide has IDI polypeptide activity by measuring the ability of the polypeptide to interconvert IPP and DMAPP in vitro, in a cell extract, or in vivo. Exemplary IDI nucleic acids include nucleic acids that encode a polypeptide, fragment of a polypeptide, peptide, or fusion polypeptide that has at least one activity of an IDI polypeptide. Exemplary IDI polypeptides and nucleic acids include naturally-occurring polypeptides and nucleic acids from any of the source organisms described herein as well as mutant polypeptides and nucleic acids derived from any of the source organisms described herein.

Lower MVA pathway polypeptides include polypeptides, fragments of polypeptides, peptides, and fusions polypeptides that have at least one activity of a lower MVA pathway polypeptide. Exemplary lower MVA pathway nucleic acids include nucleic acids that encode a polypeptide, fragment of a polypeptide, peptide, or fusion polypeptide that has at least one activity of a lower MVA pathway polypeptide. Exemplary lower MVA pathway polypeptides and nucleic acids include naturally-occurring polypeptides and nucleic acids from any of the source organisms described herein. In addition, variants of lower MVA pathway polypeptides that confer the result of better isoprene production can also be used as well.

In some aspects, the lower MVA pathway polypeptide is a polypeptide from Saccharomyces cerevisiae, Enterococcus faecalis, or Methanosarcina mazei. In some aspects, the MVK polypeptide is selected from the group consisting of Lactobacillus mevalonate kinase polypeptide, Lactobacillus sakei mevalonate kinase polypeptide, yeast mevalonate kinase polypeptide, Saccharomyces cerevisiae mevalonate kinase polypeptide, Streptococcus mevalonate kinase polypeptide, Streptococcus pneumoniae mevalonate kinase polypeptide, Streptomyces mevalonate kinase polypeptide, Streptomyces CL190 mevalonate kinase polypeptide, and Methanosarcina mazei mevalonate kinase polypeptide. Any one of the promoters described herein (e.g., promoters described herein and identified in the Examples of the present disclosure including inducible promoters and constitutive promoters) can be used to drive expression of any of the MVA polypeptides described herein.

Isoprene Synthase—Nucleic Acids and Polypeptides

In some aspects of the invention, the recombinant cells described in any of the compositions or methods described herein further comprise one or more nucleic acids encoding an isoprene synthase polypeptide or a polypeptide having isoprene synthase activity. In some aspects, the isoprene synthase polypeptide is an endogenous polypeptide. In some aspects, the endogenous nucleic acid encoding an isoprene synthase polypeptide is operably linked to a constitutive promoter. In some aspects, the endogenous nucleic acid encoding an isoprene synthase polypeptide is operably linked to an inducible promoter. In some aspects, the endogenous nucleic acid encoding an isoprene synthase polypeptide is operably linked to a strong promoter. In some aspects, more than one endogenous nucleic acid encoding an isoprene synthase polypeptide is used (e.g, 2, 3, 4, or more copies of an endogenous nucleic acid encoding an isoprene synthase polypeptide). In a particular aspect, the cells are engineered to overexpress the endogenous isoprene synthase pathway polypeptide relative to wild-type cells. In some aspects, the endogenous nucleic acid encoding an isoprene synthase polypeptide is operably linked to a weak promoter. In some aspects, the isoprene synthase polypeptide is a polypeptide from Pueraria or Populus or a hybrid such as Populus alba×Populus tremula.

In some aspects, the isoprene synthase polypeptide is a heterologous polypeptide. In some aspects, the cells comprise more than one copy of a heterologous nucleic acid encoding an isoprene synthase polypeptide. In some aspects, the heterologous nucleic acid encoding an isoprene synthase polypeptide is operably linked to a constitutive promoter. In some aspects, the heterologous nucleic acid encoding an isoprene synthase polypeptide is operably linked to an inducible promoter. In some aspects, the heterologous nucleic acid encoding an isoprene synthase polypeptide is operably linked to a strong promoter. In some aspects, the heterologous nucleic acid encoding an isoprene synthase polypeptide is operably linked to a weak promoter.

The nucleic acids encoding an isoprene synthase polypeptide(s) can be integrated into a genome of the host cells or can be stably expressed in the cells. The nucleic acids encoding an isoprene synthase polypeptide(s) can additionally be on a vector.

Exemplary isoprene synthase nucleic acids include nucleic acids that encode a polypeptide, fragment of a polypeptide, peptide, or fusion polypeptide that has at least one activity of an isoprene synthase polypeptide. Isoprene synthase polypeptides convert dimethylallyl diphosphate (DMAPP) into isoprene. Exemplary isoprene synthase polypeptides include polypeptides, fragments of polypeptides, peptides, and fusions polypeptides that have at least one activity of an isoprene synthase polypeptide. Exemplary isoprene synthase polypeptides and nucleic acids include naturally-occurring polypeptides and nucleic acids from any of the source organisms described herein. In addition, variants of isoprene synthase can possess improved activity such as improved enzymatic activity. In some aspects, an isoprene synthase variant has other improved properties, such as improved stability (e.g., thermo-stability), and/or improved solubility.

Standard methods can be used to determine whether a polypeptide has isoprene synthase polypeptide activity by measuring the ability of the polypeptide to convert DMAPP into isoprene in vitro, in a cell extract, or in vivo. Isoprene synthase polypeptide activity in the cell extract can be measured, for example, as described in Silver et al., J. Biol. Chem. 270:13010-13016, 1995. In one exemplary assay, DMAPP (Sigma) can be evaporated to dryness under a stream of nitrogen and rehydrated to a concentration of 100 mM in 100 mM potassium phosphate buffer pH 8.2 and stored at −20° C. To perform the assay, a solution of 5 μL of 1M MgCl₂, 1 mM (250 μg/ml) DMAPP, 65 μL of Plant Extract Buffer (PEB) (50 mM Tris-HCl, pH 8.0, 20 mM MgCl₂, 5% glycerol, and 2 mM DTT) can be added to 25 μL of cell extract in a 20 ml Headspace vial with a metal screw cap and teflon coated silicon septum (Agilent Technologies) and cultured at 37° C. for 15 minutes with shaking. The reaction can be quenched by adding 200 μL of 250 mM EDTA and quantified by GC/MS.

In some aspects, the isoprene synthase polypeptide is a plant isoprene synthase polypeptide or a variant thereof. In some aspects, the isoprene synthase polypeptide is an isoprene synthase from Pueraria or a variant thereof. In some aspects, the isoprene synthase polypeptide is an isoprene synthase from Populus or a variant thereof. In some aspects, the isoprene synthase polypeptide is a poplar isoprene synthase polypeptide or a variant thereof. In some aspects, the isoprene synthase polypeptide is a kudzu isoprene synthase polypeptide or a variant thereof. In some aspects, the isoprene synthase polypeptide is a polypeptide from Pueraria or Populus or a hybrid, Populus alba×Populus tremula, or a variant thereof.

In some aspects, the isoprene synthase polypeptide or nucleic acid is from the family Fabaceae, such as the Faboideae subfamily. In some aspects, the isoprene synthase polypeptide or nucleic acid is a polypeptide or nucleic acid from Pueraria montana (kudzu) (Sharkey et al., Plant Physiology 137: 700-712, 2005), Pueraria lobata, poplar (such as Populus alba, Populus nigra, Populus trichocarpa, or Populus alba×tremula (CAC35696) (Miller et al., Planta 213: 483-487, 2001), aspen (such as Populus tremuloides) (Silver et al., JBC 270(22): 13010-1316, 1995), English Oak (Quercus robur) (Zimmer et al., WO 98/02550), or a variant thereof. In some aspects, the isoprene synthase polypeptide is an isoprene synthase from Pueraria montana, Pueraria lobata, Populus tremuloides, Populus alba, Populus nigra, or Populus trichocarpa or a variant thereof. In some aspects, the isoprene synthase polypeptide is an isoprene synthase from Populus alba or a variant thereof. In some aspects, the nucleic acid encoding the isoprene synthase (e.g., isoprene synthase from Populus alba or a variant thereof) is codon optimized.

In some aspects, the isoprene synthase nucleic acid or polypeptide is a naturally-occurring polypeptide or nucleic acid (e.g., naturally-occurring polypeptide or nucleic acid from Populus). In some aspects, the isoprene synthase nucleic acid or polypeptide is not a wild-type or naturally-occurring polypeptide or nucleic acid. In some aspects, the isoprene synthase nucleic acid or polypeptide is a variant of a wild-type or naturally-occurring polypeptide or nucleic acid (e.g., a variant of a wild-type or naturally-occurring polypeptide or nucleic acid from Populus).

In some aspects, the isoprene synthase polypeptide is a variant. In some aspects, the isoprene synthase polypeptide is a variant of a wild-type or naturally occurring isoprene synthase. In some aspects, the variant has improved activity such as improved catalytic activity compared to the wild-type or naturally occurring isoprene synthase. The increase in activity (e.g., catalytic activity) can be at least about any of 10%, 20%, 30%, 40%, 50%, 60%, 70%, 80%, 90%, or 95%. In some aspects, the increase in activity such as catalytic activity is at least about any of 1 fold, 2 folds, 5 folds, 10 folds, 20 folds, 30 folds, 40 folds, 50 folds, 75 folds, or 100 folds. In some aspects, the increase in activity such as catalytic activity is about 10% to about 100 folds (e.g., about 20% to about 100 folds, about 50% to about 50 folds, about 1 fold to about 25 folds, about 2 folds to about 20 folds, or about 5 folds to about 20 folds). In some aspects, the variant has improved solubility compared to the wild-type or naturally occurring isoprene synthase. The increase in solubility can be at least about any of 10%, 20%, 30%, 40%, 50%, 60%, 70%, 80%, 90%, or 95%. The increase in solubility can be at least about any of 1 fold, 2 folds, 5 folds, 10 folds, 20 folds, 30 folds, 40 folds, 50 folds, 75 folds, or 100 folds. In some aspects, the increase in solubility is about 10% to about 100 folds (e.g., about 20% to about 100 folds, about 50% to about 50 folds, about 1 fold to about 25 folds, about 2 folds to about 20 folds, or about 5 folds to about 20 folds). In some aspects, the isoprene synthase polypeptide is a variant of naturally occurring isoprene synthase and has improved stability (such as thermo-stability) compared to the naturally occurring isoprene synthase.

In some aspects, the variant has at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200% of the activity of a wild-type or naturally occurring isoprene synthase. The variant can share sequence similarity with a wild-type or naturally occurring isoprene synthase. In some aspects, a variant of a wild-type or naturally occurring isoprene synthase can have at least about any of 40%, 50%, 60%, 70%, 75%, 80%, 90%, 91%, 92%, 93%, 94%, 95%, 96%, 97%, 98%, 99%, 99.5%, or 99.9% amino acid sequence identity as that of the wild-type or naturally occurring isoprene synthase. In some aspects, a variant of a wild-type or naturally occurring isoprene synthase has any of about 70% to about 99.9%, about 75% to about 99%, about 80% to about 98%, about 85% to about 97%, or about 90% to about 95% amino acid sequence identity as that of the wild-type or naturally occurring isoprene synthase.

In some aspects, the variant comprises a mutation in the wild-type or naturally occurring isoprene synthase. In some aspects, the variant has at least one amino acid substitution, at least one amino acid insertion, and/or at least one amino acid deletion. In some aspects, the variant has at least one amino acid substitution. In some aspects, the number of differing amino acid residues between the variant and wild-type or naturally occurring isoprene synthase can be one or more, e.g. 1, 2, 3, 4, 5, 10, 15, 20, 30, 40, 50, or more amino acid residues. Naturally occurring isoprene synthases can include any isoprene synthases from plants, for example, kudzu isoprene synthases, poplar isoprene synthases, English oak isoprene synthases, and willow isoprene synthases. In some aspects, the variant is a variant of isoprene synthase from Populus alba. In some aspects, the variant of isoprene synthase from Populus alba has at least one amino acid substitution, at least one amino acid insertion, and/or at least one amino acid deletion. In some aspects, the variant is a truncated Populus alba isoprene synthase. In some aspects, the nucleic acid encoding variant (e.g., variant of isoprene synthase from Populus alba) is codon optimized (for example, codon optimized based on host cells where the heterologous isoprene synthase is expressed).

The isoprene synthase polypeptide provided herein can be any of the isoprene synthases or isoprene synthase variants described in WO 2009/132220, WO 2010/124146, WO 2012/058494, and U.S. Patent Application Publication No.: 2010/0086978, the contents of which are expressly incorporated herein by reference in their entirety with respect to the isoprene synthases and isoprene synthase variants.

Any one of the promoters described herein (e.g., promoters described herein and identified in the Examples of the present disclosure including inducible promoters and constitutive promoters) can be used to drive expression of any of the isoprene synthases described herein.

Suitable isoprene synthases include, but are not limited to, those identified by Genbank Accession Nos. AY341431, AY316691, AY279379, AJ457070, and AY182241. Types of isoprene synthases which can be used in any one of the compositions or methods including methods of making microorganisms encoding isoprene synthase described herein are also described in International Patent Application Publication Nos. WO2009/076676, WO2010/003007, WO2009/132220, WO2010/031062, WO2010/031068, WO2010/031076, WO2010/013077, WO2010/031079, WO2010/148150, WO2010/124146, WO2010/078457, WO2010/148256 and WO 2012/058494, the contents of which are expressly incorporated herein by reference in their entirety with respect to the isoprene synthases and isoprene synthase variants.

Nucleic Acids Encoding DXP Pathway Polypeptides

In some aspects of the invention, the recombinant cells described in any of the compositions or methods described herein further comprise one or more heterologous nucleic acids encoding a DXS polypeptide or other DXP pathway polypeptides. In some aspects, the cells further comprise a chromosomal copy of an endogenous nucleic acid encoding a DXS polypeptide or other DXP pathway polypeptides. In some aspects, the E. coli cells further comprise one or more nucleic acids encoding an IDI polypeptide and a DXS polypeptide or other DXP pathway polypeptides. In some aspects, one nucleic acid encodes the isoprene synthase polypeptide, IDI polypeptide, and DXS polypeptide or other DXP pathway polypeptides. In some aspects, one plasmid encodes the isoprene synthase polypeptide, IDI polypeptide, and DXS polypeptide or other DXP pathway polypeptides. In some aspects, multiple plasmids encode the isoprene synthase polypeptide, IDI polypeptide, and DXS polypeptide or other DXP pathway polypeptides.

Exemplary DXS polypeptides include polypeptides, fragments of polypeptides, peptides, and fusions polypeptides that have at least one activity of a DXS polypeptide. Standard methods (such as those described herein) can be used to determine whether a polypeptide has DXS polypeptide activity by measuring the ability of the polypeptide to convert pyruvate and D-glyceraldehyde-3-phosphate into 1-deoxy-D-xylulose-5-phosphate in vitro, in a cell extract, or in vivo. Exemplary DXS polypeptides and nucleic acids and methods of measuring DXS activity are described in more detail in International Publication No. WO 2009/076676, U.S. patent application Ser. No. 12/335,071 (US Publ. No. 2009/0203102), WO 2010/003007, US Publ. No. 2010/0048964, WO 2009/132220, and US Publ. No. 2010/0003716.

Exemplary DXP pathways polypeptides include, but are not limited to any of the following polypeptides: DXS polypeptides, DXR polypeptides, MCT polypeptides, CMK polypeptides, MCS polypeptides, HDS polypeptides, HDR polypeptides, and polypeptides (e.g., fusion polypeptides) having an activity of one, two, or more of the DXP pathway polypeptides. In particular, DXP pathway polypeptides include polypeptides, fragments of polypeptides, peptides, and fusions polypeptides that have at least one activity of a DXP pathway polypeptide. Exemplary DXP pathway nucleic acids include nucleic acids that encode a polypeptide, fragment of a polypeptide, peptide, or fusion polypeptide that has at least one activity of a DXP pathway polypeptide. Exemplary DXP pathway polypeptides and nucleic acids include naturally-occurring polypeptides and nucleic acids from any of the source organisms described herein as well as mutant polypeptides and nucleic acids derived from any of the source organisms described herein. Exemplary DXP pathway polypeptides and nucleic acids and methods of measuring DXP pathway polypeptide activity are described in more detail in International Publication No.: WO 2010/148150

Exemplary DXS polypeptides include polypeptides, fragments of polypeptides, peptides, and fusions polypeptides that have at least one activity of a DXS polypeptide. Standard methods (such as those described herein) can be used to determine whether a polypeptide has DXS polypeptide activity by measuring the ability of the polypeptide to convert pyruvate and D-glyceraldehyde-3-phosphate into 1-deoxy-D-xylulose-5-phosphate in vitro, in a cell extract, or in vivo. Exemplary DXS polypeptides and nucleic acids and methods of measuring DXS activity are described in more detail in International Publication No. WO 2009/076676, U.S. patent application Ser. No. 12/335,071 (US Publ. No. 2009/0203102), WO 2010/003007, US Publ. No. 2010/0048964, WO 2009/132220, and US Publ. No. 2010/0003716.

In particular, DXS polypeptides convert pyruvate and D-glyceraldehyde 3-phosphate into 1-deoxy-d-xylulose 5-phosphate (DXP). Standard methods can be used to determine whether a polypeptide has DXS polypeptide activity by measuring the ability of the polypeptide to convert pyruvate and D-glyceraldehyde 3-phosphate in vitro, in a cell extract, or in vivo.

DXR polypeptides convert 1-deoxy-d-xylulose 5-phosphate (DXP) into 2-C-methyl-D-erythritol 4-phosphate (MEP). Standard methods can be used to determine whether a polypeptide has DXR polypeptides activity by measuring the ability of the polypeptide to convert DXP in vitro, in a cell extract, or in vivo.

MCT polypeptides convert 2-C-methyl-D-erythritol 4-phosphate (MEP) into 4-(cytidine 5′-diphospho)-2-methyl-D-erythritol (CDP-ME). Standard methods can be used to determine whether a polypeptide has MCT polypeptides activity by measuring the ability of the polypeptide to convert MEP in vitro, in a cell extract, or in vivo.

CMK polypeptides convert 4-(cytidine 5′-diphospho)-2-C-methyl-D-erythritol (CDP-ME) into 2-phospho-4-(cytidine 5′-diphospho)-2-C-methyl-D-erythritol (CDP-MEP). Standard methods can be used to determine whether a polypeptide has CMK polypeptides activity by measuring the ability of the polypeptide to convert CDP-ME in vitro, in a cell extract, or in vivo.

MCS polypeptides convert 2-phospho-4-(cytidine 5′-diphospho)-2-C-methyl-D-erythritol (CDP-MEP) into 2-C-methyl-D-erythritol 2,4-cyclodiphosphate (ME-CPP or cMEPP). Standard methods can be used to determine whether a polypeptide has MCS polypeptides activity by measuring the ability of the polypeptide to convert CDP-MEP in vitro, in a cell extract, or in vivo.

HDS polypeptides convert 2-C-methyl-D-erythritol 2,4-cyclodiphosphate into (E)-4-hydroxy-3-methylbut-2-en-1-yl diphosphate (HMBPP or HDMAPP). Standard methods can be used to determine whether a polypeptide has HDS polypeptides activity by measuring the ability of the polypeptide to convert ME-CPP in vitro, in a cell extract, or in vivo.

HDR polypeptides convert (E)-4-hydroxy-3-methylbut-2-en-1-yl diphosphate into isopentenyl diphosphate (IPP) and dimethylallyl diphosphate (DMAPP). Standard methods can be used to determine whether a polypeptide has HDR polypeptides activity by measuring the ability of the polypeptide to convert HMBPP in vitro, in a cell extract, or in vivo.

### Source Organisms for MVA Pathway, Isoprene Synthase, IDI, and DXP Pathway Polypeptides

Isoprene synthase, IDI, DXP pathway, and/or MVA pathway nucleic acids (excluding enzymes that condense two acetoacetyl-CoA molecules to acetyl-CoA, such as acetoacetyl-CoA thiolase or AACT), MVA pathway polypeptides (excluding enzymes that condense two acetoacetyl-CoA molecules to acetyl-CoA, such as AACT) can be obtained from any organism that naturally contains isoprene synthase, IDI, DXP pathway, and/or MVA pathway nucleic acids. Isoprene is formed naturally by a variety of organisms, such as bacteria, yeast, plants, and animals. Some organisms contain the MVA pathway for producing isoprene. Isoprene synthase nucleic acids can be obtained, e.g., from any organism that contains an isoprene synthase. MVA pathway nucleic acids can be obtained, e.g., from any organism that contains the MVA pathway. IDI and DXP pathway nucleic acids can be obtained, e.g., from any organism that contains the IDI and DXP pathway.

The nucleic acid sequence of the isoprene synthase, DXP pathway, IDI, and/or MVA pathway nucleic acids can be isolated from a bacterium, fungus, plant, algae, or cyanobacterium. Exemplary source organisms include, for example, yeasts, such as species of Saccharomyces (e.g., S. cerevisiae), bacteria, such as species of Escherichia (e.g., E. coli), or species of Methanosarcina (e.g., Methanosarcina mazei), plants, such as kudzu or poplar (e.g., Populus alba or Populus alba×tremula CAC35696) or aspen (e.g., Populus tremuloides). Exemplary sources for isoprene synthases, IDI, and/or MVA pathway polypeptides which can be used are also described in International Patent Application Publication Nos. WO2009/076676, WO2010/003007, WO2009/132220, WO2010/031062, WO2010/031068, WO2010/031076, WO2010/013077, WO2010/031079, WO2010/148150, WO2010/078457, and WO2010/148256.

In some aspects, the source organism is a yeast, such as Saccharomyces sp., Schizosaccharomyces sp., Pichia sp., or Candida sp.

In some aspects, the source organism is a bacterium, such as strains of Bacillus such as B. lichenformis or B. subtilis, strains of Pantoea such as P. citrea, strains of Pseudomonas such as P. alcaligenes, strains of Streptomyces such as S. lividans or S. rubiginosus, strains of Escherichia such as E. coli, strains of Enterobacter, strains of Streptococcus, or strains of Archaea such as Methanosarcina mazei.

As used herein, “the genus Bacillus” includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

In some aspects, the source organism is a gram-positive bacterium. Non-limiting examples include strains of Streptomyces (e.g., S. lividans, S. coelicolor, or S. griseus) and Bacillus. In some aspects, the source organism is a gram-negative bacterium, such as E. coli or Pseudomonas sp. In some aspects, the source organism is L. acidophilus.

In some aspects, the source organism is a plant, such as a plant from the family Fabaceae, such as the Faboideae subfamily. In some aspects, the source organism is kudzu, poplar (such as Populus alba×tremula CAC35696), aspen (such as Populus tremuloides), or Quercus robur.

In some aspects, the source organism is an algae, such as a green algae, red algae, glaucophytes, chlorarachniophytes, euglenids, chromista, or dinoflagellates.

In some aspects, the source organism is a cyanobacteria, such as cyanobacteria classified into any of the following groups based on morphology: Chroococcales, Pleurocapsales, Oscillatoriales, Nostocales, or Stigonematales.

Exemplary Host Cells

One of skill in the art will recognize that expression vectors are designed to contain certain components which optimize gene expression for certain host strains. Such optimization components include, but are not limited to origin of replication, promoters, and enhancers. The vectors and components referenced herein are described for exemplary purposes and are not meant to narrow the scope of the invention.

Any microorganism or progeny thereof can be used to express any of the genes (heterologous or endogenous) described herein. Bacteria cells, including gram positive or gram negative bacteria can be used to express any of the genes described herein. In particular, the genes described herein can be expressed in any one of the group consisting of Escherichia sp. (e.g., E. coli), L. acidophilus, P. citrea, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, Corynebacterium spp. (e.g., C. glutamicum), S. degradans 2-40, Alginovibrio aqualiticus, Alteromonas sr. strain KLIA, Asteromyces cruciatus, Beneckea pelagia, Enterobacter cloacae, Halmonas marina, Klebsiella pneumonia, Photobacterium spp. (ATCC 433367), Pseudoalteromonas elyakovii, Pseudomonas sp. (e.g., Pseudomonas alginovora, Pseudomonas aeruginosa, Pseudomonas maltophilia, Pseudomonas putida), Vibrio alginolyticus, Vibrio halioticol, and Vibrio harveyi, S. albus, S. lividans, S. coelicolor, S. griseus, and P. alcaligenes cells. In one aspect, the bacterial cell is an E. coli cell. In another aspect, the bacterial cell is an L. acidophilus cell. There are numerous types of anaerobic cells that can be used as host cells in the compositions and methods of the present invention. In one aspect of the invention, the cells described in any of the compositions or methods described herein are obligate anaerobic cells and progeny thereof. Obligate anaerobes typically do not grow well, if at all, in conditions where oxygen is present. It is to be understood that a small amount of oxygen may be present, that is, there is some tolerance level that obligate anaerobes have for a low level of oxygen. In one aspect, obligate anaerobes engineered to produce mevalonate, isoprene, isoprenoid precursors, and isoprenoids can serve as host cells for any of the methods and/or compositions described herein and are grown under substantially oxygen-free conditions, wherein the amount of oxygen present is not harmful to the growth, maintenance, and/or fermentation of the anaerobes.

In another aspect of the invention, the host cells described and/or used in any of the compositions or methods described herein are facultative anaerobic cells and progeny thereof. Facultative anaerobes can generate cellular ATP by aerobic respiration (e.g., utilization of the TCA cycle) if oxygen is present. However, facultative anaerobes can also grow in the absence of oxygen. This is in contrast to obligate anaerobes which die or grow poorly in the presence of greater amounts of oxygen. In one aspect, therefore, facultative anaerobes can serve as host cells for any of the compositions and/or methods provided herein and can be engineered to produce mevalonate, isoprene, isoprenoid precursors, and isoprenoids. Facultative anaerobic host cells can be grown under substantially oxygen-free conditions, wherein the amount of oxygen present is not harmful to the growth, maintenance, and/or fermentation of the anaerobes, or can be alternatively grown in the presence of greater amounts of oxygen.

The host cell can additionally be a filamentous fungal cell and progeny thereof. (See, e.g., Berka & Barnett, Biotechnology Advances, (1989), 7(2):127-154). In some aspects, the filamentous fungal cell can be any of Trichoderma longibrachiatum, T. viride, T. koningii, T. harzianum, Penicillium sp., Humicola insolens, H. lanuginose, H. grisea, Chrysosporium sp., C. lucknowense, Gliocladium sp., Aspergillus sp., such as A. oryzae, A. niger, A sojae, A. japonicus, A. nidulans, or A. awamori, Fusarium sp., such as F. roseum, F. graminum F. cerealis, F. oxysporuim, or F. venenatum, Neurospora sp., such as N. crassa, Hypocrea sp., Mucor sp., such as M. miehei, Rhizopus sp. or Emericella sp. In some aspects, the fungus is A. nidulans, A. awamori, A. oryzae, A. aculeatus, A. niger, A. japonicus, T. reesei, T. viride, F. oxysporum, or F. solani. In certain embodiments, plasmids or plasmid components for use herein include those described in U.S. patent pub. No. US 2011/0045563.

The host cell can also be a yeast, such as Saccharomyces sp., Schizosaccharomyces sp., Pichia sp., or Candida sp. In some aspects, the Saccharomyces sp. is Saccharomyces cerevisiae (See, e.g., Romanos et al., Yeast, (1992), 8(6):423-488). In certain embodiments, plasmids or plasmid components for use herein include those described in U.S. Pat. No. 7,659,097 and U.S. patent pub. No. US 2011/0045563.

The host cell can additionally be a species of algae, such as a green algae, red algae, glaucophytes, chlorarachniophytes, euglenids, chromista, or dinoflagellates. (See, e.g., Saunders & Warmbrodt, “Gene Expression in Algae and Fungi, Including Yeast,” (1993), National Agricultural Library, Beltsville, Md.). In certain embodiments, plasmids or plasmid components for use herein include those described in U.S. Patent Pub. No. US 2011/0045563. In some aspects, the host cell is a cyanobacterium, such as cyanobacterium classified into any of the following groups based on morphology: Chlorococcales, Pleurocapsales, Oscillatoriales, Nostocales, or Stigonematales (See, e.g., Lindberg et al., Metab. Eng., (2010) 12(1):70-79). In certain embodiments, plasmids or plasmid components for use herein include those described in U.S. patent pub. No. US 2010/0297749; US 2009/0282545 and Intl. Pat. Appl. No. WO 2011/034863.

In certain embodiments, E. coli host cells can be used to express any of the genes described herein. In one aspect, the host cell is a recombinant cell of an Escherichia coli (E. coli) strain, or progeny thereof, capable of producing mevalonate that expresses one or more nucleic acids encoding an acetoacetyl-CoA synthase. The E. coli host cells can produce isoprene, isoprenoid precursors (e.g., mevalonate), and/or isoprenoids in amounts, peak titers, and cell productivities greater than that of the same cells lacking one or more heterologously expressed nucleic acids encoding an acetoacetyl-CoA synthase. In addition, the one or more heterologously expressed nucleic acids encoding an acetoacetyl-CoA synthase in E. coli can be chromosomal copies (e.g., integrated into the E. coli chromosome). In other aspects, the E. coli cells are in culture.

### Transformation Methods

Nucleic acids encoding acetoacetyl-CoA synthase, an enzyme that produces acetoacetyl-CoA synthase from malonyl-CoA and acetyl-CoA, non-thiolase MVA pathway polypeptides, DXP pathway polypeptides, isoprene synthase, IDI, polyprenyl pyrophosphate synthases and any other enzyme needed to produce isoprene, isoprenoid precursors, and/or isoprenoids can be introduced into host cells (e.g., a plant cell, a fungal cell, a yeast cell, or a bacterial cell) by any technique known to one of the skill in the art.

Standard techniques for introduction of a DNA construct or vector into a host cell, such as transformation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion can be used. General transformation techniques are known in the art (See, e.g., Current Protocols in Molecular Biology (F. M. Ausubel et al. (eds.) Chapter 9, 1987; Sambrook et al., Molecular Cloning: A Laboratory Manual, 3r^{d} ed., Cold Spring Harbor, 2001; and Campbell et al., Curr. Genet. 16:53-56, 1989). The introduced nucleic acids can be integrated into chromosomal DNA or maintained as extrachromosomal replicating sequences. Transformants can be selected by any method known in the art. Suitable methods for selecting transformants are described in International Publication No. WO 2009/076676, U.S. patent application Ser. No. 12/335,071 (US Publ. No. 2009/0203102), WO 2010/003007, US Publ. No. 2010/0048964, WO 2009/132220, and US Publ. No. 2010/0003716.

In one embodiment, a bacterium such as Escherichia coli is used as a host. In this embodiment, an expression vector can be selected and/or engineered to be able to autonomously replicate in such bacterium. Promoters, a ribosome binding sequence, transcription termination sequence(s) can also be included in the expression vector, in addition to the genes listed herein. Optionally, an expression vector may contain a gene that controls promoter activity.

Any promoter may be used as long as it can be expressed in a host such as Escherichia coli. Examples of such promoter that can be used include a trp promoter, an lac promoter, a PL promoter, a PR promoter, and the like from Escherichia coli, and a T7 promoter from a phage. Further, an artificially designed or modified promoter such as a tac promoter may be used.

A method for introduction of an expression vector is not particularly limited as long as DNA is introduced into a bacterium thereby. Examples thereof include a method using calcium ions (Cohen, S, N., et al.: Proc. Natl. Acad. Sci., USA, 69:2110-2114 (1972)) and an electroporation method.

When a yeast is used as a host, Saccharomyces cerevisiae, Schizosaccharomyces pombe, Pichia pastoris, or the like can be used. In this case, a promoter is not particularly limited as long as it can be expressed in yeast. Examples thereof include a gall promoter, a gal10 promoter, a heat-shock protein promoter, an MF.alpha.1 promoter, a PHO5 promoter, a PGK promoter, a GAP promoter, an ADH promoter, and an AOX1 promoter.

A method for introducing a recombinant vector into yeast is not particularly limited as long as DNA is introduced into yeast thereby. Examples thereof include the electroporation method (Becker, D. M., et al. Methods. Enzymol., 194: 182-187 (1990)), the spheroplast method (Hinnen, A. et al.: Proc. Natl. Acad. Sci., USA, 75: 1929-1933 (1978)), and the lithium acetate method (Itoh, H.: J. Bacteriol., 153: 163-168 (1983)).

In particular, it is preferable to use, as a host microorganism, a microorganism with a relatively high malonyl-CoA content. Malonyl-CoA is a substance used for biosynthesis of fatty acid and is present in all microorganisms. The aforementioned acetoacetyl-CoA synthase synthesizes acetoacetyl-CoA from malonyl-CoA and acetyl-CoA. Therefore, the isoprene/isoprenoid productivity can be improved with the use of a host microorganism with a high malonyl-CoA content.

### Vectors

Suitable vectors can be used for any of the compositions and methods described herein. For example, suitable vectors can be used to optimize the expression of one or more copies of a gene encoding a HMG-CoA reductase, an isoprene synthase, a polyprenyl pyrophosphate synthase, and/or one or more non-thiolase MVA pathway polypeptides. In some aspects, the vector contains a selective marker. Examples of selectable markers include, but are not limited to, antibiotic resistance nucleic acids (e.g., kanamycin, ampicillin, carbenicillin, gentamicin, hygromycin, phleomycin, bleomycin, neomycin, or chloramphenicol) and/or nucleic acids that confer a metabolic advantage, such as a nutritional advantage on the host cell. In some aspects, one or more copies of HMG-CoA reductase, an isoprene synthase, a polyprenyl pyrophosphate synthase, and/or one or more non-thiolase MVA pathway polypeptides nucleic acid(s) integrate into the genome of host cells without a selective marker. Any one of the vectors characterized or used in the Examples of the present disclosure can be used.

### Host Cell Mutations

The invention is further directed to the use of host microorganisms having mutations that increase the intracellular pool of starting malonyl-CoA. These modified host strains provide increased substrate availability (e.g., malonyl-CoA) for acetoacetyl-CoA synthase which can result in increased production of acetoacetyl-CoA and its downstream products such as isoprene and/or isoprenoids. In certain embodiments, the host microorganism can comprise genetic manipulations which attenuate or delete the activity of the citric cycle genes cycle genes sdhCDAB and citE, the amino acid transporter brnQ, and the pyruvate consumer adhE. In other embodiments, the host microorganism can comprise genetic manipulations which result in the over-expression of one or more genes, including but not limited to, acetyl-CoA carboxylase (ACC), phosphoglycerate kinase (PGK), glyceraldehyde-3-phosphatedehydrogenase (GAPD) and/or pyruvate dehydrogenase complex (PDH) thereby leading to increased intracellular malonyl-CoA levels. See, for example Fowler, et al., Applied and Environmental Microbiology, Vol. 75, No. 18, pp. 5831-5839 (2009), Zha et al., Metabolic Engineering, 11: 192-198 (2009), Xu et al., Metabolic Engineering, (2011)doi:10.1016/j.ymben.2011.06.008, Okamura et al., PNAS 107: 11265-11270 (2010), and US 2010/0285549, the contents of which are expressly incorporated herein by reference in their entirety.

The invention also contemplates additional host cell mutations that increase carbon flux through the MVA pathway. By increasing the carbon flow, more isoprene, isoprenoid precursor and/or isoprenoid can be produced. The recombinant cells comprising acetoacetyl-CoA synthase as described herein can also be engineered for increased carbon flux towards mevalonate production wherein the activity of one or more enzymes from the group consisting of: (a) citrate synthase, (b) phosphotransacetylase; (c) acetate kinase; (d) lactate dehydrogenase; (e) NADP-dependent malic enzyme, and; (f) pyruvate dehydrogenase is modulated.

Citrate Synthase Pathway

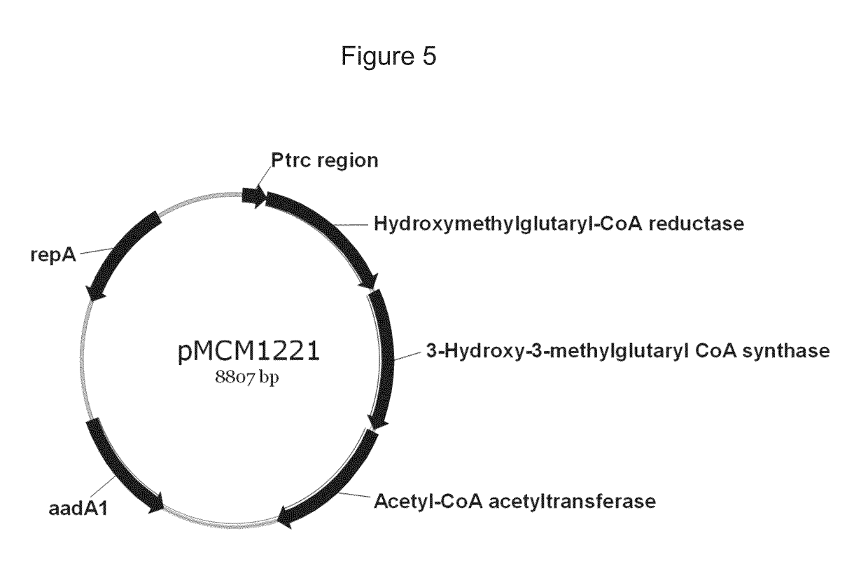

Citrate synthase catalyzes the condensation of oxaloacetate and acetyl-CoA to form citrate, a metabolite of the Tricarboxylic acid (TCA) cycle (Ner, S. et al. 1983. Biochemistry 22: 5243-5249; Bhayana, V. and Duckworth, H. 1984. Biochemistry 23: 2900-2905) (FIG. 5). In E. coli, this enzyme, encoded by gltA, behaves like a trimer of dimeric subunits. The hexameric form allows the enzyme to be allosterically regulated by NADH. This enzyme has been widely studied (Wiegand, G., and Remington, S. 1986. Annual Rev. Biophysics Biophys. Chem. 15: 97-117; Duckworth et al. 1987. Biochem Soc Symp. 54:83-92; Stockell, D. et al. 2003. J. Biol. Chem. 278: 35435-43; Maurus, R. et al. 2003. Biochemistry. 42:5555-5565). To avoid allosteric inhibition by NADH, replacement by or supplementation with the Bacillus subtilis NADH-insensitive citrate synthase has been considered (Underwood et al. 2002. Appl. Environ. Microbiol. 68:1071-1081; Sanchez et al. 2005. Met. Eng. 7:229-239).

The reaction catalyzed by citrate synthase is directly competing with the thiolase catalyzing the first step of the mevalonate pathway, as they both have acetyl-CoA as a substrate (Hedl et al. 2002. J. Bact. 184:2116-2122). Therefore, one of skill in the art can modulate citrate synthase expression (e.g., decrease enzyme activity) to allow more carbon to flux into the mevalonate pathway, thereby increasing the eventual production of mevalonate, isoprene and isoprenoids. Decrease of citrate synthase activity can be any amount of reduction of specific activity or total activity as compared to when no manipulation has been effectuated. In some instances, the decrease of enzyme activity is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%. In some aspects, the activity of citrate synthase is modulated by decreasing the activity of an endogenous citrate synthase gene. This can be accomplished by chromosomal replacement of an endogenous citrate synthase gene with a transgene encoding an NADH-insensitive citrate synthase or by using a transgene encoding an NADH-insensitive citrate synthase that is derived from Bacillus subtilis. The activity of citrate synthase can also be modulated (e.g., decreased) by replacing the endogenous citrate synthase gene promoter with a synthetic constitutively low expressing promoter. The decrease of the activity of citrate synthase can result in more carbon flux into the mevalonate dependent biosynthetic pathway in comparison to microorganisms that do not have decreased expression of citrate synthase.

Pathways Involving Phosphotransacetylase and/or Acetate Kinase

Phosphotransacetylase (pta) (Shimizu et al. 1969. Biochim. Biophys. Acta 191: 550-558) catalyzes the reversible conversion between acetyl-CoA and acetylphosphate (acetyl-P), while acetate kinase (ackA) (Kakuda, H. et al. 1994. J. Biochem. 11:916-922) uses acetyl-P to form acetate. These genes can be transcribed as an operon in E. coli. Together, they catalyze the dissimilation of acetate, with the release of ATP. Thus, one of skill in the art can increase the amount of available acetyl Co-A by attenuating the activity of phosphotransacetylase gene (e.g., the endogenous phosphotransacetylase gene) and/or an acetate kinase gene (e.g., the endogenous acetate kinase gene). One way of achieving attenuation is by deleting phosphotransacetylase (pta) and/or acetate kinase (ackA). This can be accomplished by replacing one or both genes with a chloramphenicol cassette followed by looping out of the cassette. Acetate is produced by E. coli for a variety of reasons (Wolfe, A. 2005. Microb. Mol. Biol. Rev. 69:12-50). Without being bound by theory, since ackA-pta use acetyl-CoA, deleting those genes might allow carbon not to be diverted into acetate and to increase the yield of mevalonate, isoprene or isoprenoids.

In some aspects, the recombinant microorganism produces decreased amounts of acetate in comparison to microorganisms that do not have attenuated endogenous phosphotransacetylase gene and/or endogenous acetate kinase gene expression. Decrease in the amount of acetate produced can be measured by routine assays known to one of skill in the art. The amount of acetate reduction is at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99% as compared when no molecular manipulations are done.

The activity of phosphotransacetylase (pta) and/or acetate kinase (ackA) can also be decreased by other molecular manipulation of the enzymes. The decrease of enzyme activity can be any amount of reduction of specific activity or total activity as compared to when no manipulation has been effectuated. In some instances, the decrease of enzyme activity is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%.

In some cases, attenuating the activity of the endogenous phosphotransacetylase gene and/or the endogenous acetate kinase gene results in more carbon flux into the mevalonate dependent biosynthetic pathway in comparison to microorganisms that do not have attenuated endogenous phosphotransacetylase gene and/or endogenous acetate kinase gene expression.

Pathways Involving Lactate Dehydrogenase

In E. coli, D-Lactate is produced from pyruvate through the enzyme lactate dehydrogenase (ldhA—FIG. 5) (Bunch, P. et al. 1997. Microbiol. 143:187-195). Production of lactate is accompanied with oxidation of NADH, hence lactate is produced when oxygen is limited and cannot accommodate all the reducing equivalents. Thus, production of lactate could be a source for carbon consumption. As such, to improve carbon flow through to mevolnate production (and isoprene, isoprenoid precursor and isoprenoids production, if desired), one of skill in the art can modulate the activity of lactate dehydrogenase, such as by decreasing the activity of the enzyme.

Accordingly, in one aspect, the activity of lactate dehydrogenase can be modulated by attenuating the activity of an endogenous lactate dehydrogenase gene. Such attenuation can be achieved by deletion of the endogenous lactate dehydrogenase gene. Other ways of attenuating the activity of lactate dehydrogenase gene known to one of skill in the art may also be used. By manipulating the pathway that involves lactate dehydrogenase, the recombinant microorganism produces decreased amounts of lactate in comparison to microorganisms that do not have attenuated endogenous lactate dehydrogenase gene expression. Decrease in the amount of lactate produced can be measured by routine assays known to one of skill in the art. The amount of lactate reduction is at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99% as compared when no molecular manipulations are done.

The activity of lactate dehydrogenase can also be decreased by other molecular manipulations of the enzyme. The decrease of enzyme activity can be any amount of reduction of specific activity or total activity as compared to when no manipulation has been effectuated. In some instances, the decrease of enzyme activity is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%.

Accordingly, in some cases, attenuation of the activity of the endogenous lactate dehydrogenase gene results in more carbon flux into the mevalonate dependent biosynthetic pathway in comparison to microorganisms that do not have attenuated endogenous lactate dehydrogenase gene expression.

Pathways Involving Malic Enzyme

Malic enzyme (in E. coli sfcA and maeB) is an anaplerotic enzyme that catalyzes the conversion of malate into pyruvate (using NAD+ or NADP+) by the equation below:

(S)-malate+NAD(P)⁺ [symbol] pyruvate+CO₂+NAD(P)H

Thus, the two substrates of this enzyme are (S)-malate and NAD(P)⁺, whereas its 3 products are pyruvate, CO₂, and NADPH.

Expression of the NADP-dependent malic enzyme (maeB—FIG. 5) (Iwikura, M. et al. 1979. J. Biochem. 85: 1355-1365) can help increase mevalonate, isoprene, isoprenoid precursors and isoprenoids yield by 1) bringing carbon from the TCA cycle back to pyruvate, direct precursor of acetyl-CoA, itself direct precursor of the mevalonate pathway and 2) producing extra NADPH which could be used in the HMG-CoA reductase reaction (Oh, M K et al. (2002) J. Biol. Chem. 277: 13175-13183; Bologna, F. et al. (2007) J. Bact. 189:5937-5946).

As such, more starting substrate (pyruvate or acetyl-CoA) for the downstream production of mevalonate, isoprene, isoprenoid precursors and isoprenoids can be achieved by modulating, such as increasing, the activity and/or expression of malic enzyme. The NADP-dependent malic enzyme gene can be an endogenous gene. One non-limiting way to accomplish this is by replacing the endogenous NADP-dependent malic enzyme gene promoter with a synthetic constitutively expressing promoter. Another non-limiting way to increase enzyme activity is by using one or more heterologous nucleic acids encoding an NADP-dependent malic enzyme polypeptide. One of skill in the art can monitor the expression of maeB RNA during fermentation or culturing using readily available molecular biology techniques.

Accordingly, in some embodiments, the recombinant microorganism produces increased amounts of pyruvate in comparison to microorganisms that do not have increased expression of an NADP-dependent malic enzyme gene. In some aspects, increasing the activity of an NADP-dependent malic enzyme gene results in more carbon flux into the mevalonate dependent biosynthetic pathway in comparison to microorganisms that do not have increased NADP-dependent malic enzyme gene expression.

Increase in the amount of pyruvate produced can be measured by routine assays known to one of skill in the art. The amount of pyruvate increase can be at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99% as compared when no molecular manipulations are done.

The activity of malic enzyme can also be increased by other molecular manipulations of the enzyme. The increase of enzyme activity can be any amount of increase of specific activity or total activity as compared to when no manipulation has been effectuated. In some instances, the increase of enzyme activity is at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%.

Pathways Involving Pyruvate Dehydrogenase Complex

The pyruvate dehydrogenase complex, which catalyzes the decarboxylation of pyruvate into acetyl-CoA, is composed of the proteins encoded by the genes aceE, aceF and lpdA. Transcription of those genes is regulated by several regulators. Thus, one of skill in the art can increase acetyl-CoA by modulating the activity of the pyruvate dehydrogenase complex. Modulation can be to increase the activity and/or expression (e.g., constant expression) of the pyruvate dehydrogenase complex. This can be accomplished by different ways, for example, by placing a strong constitutive promoter, like PL.6 (aattcatataaaaaacatacagataaccatctgcggtgataaattatctctggcggtgttgacataaataccactggcggtgatactgagcacatc agcaggacgcactgaccaccatgaaggtg-lambda promoter, GenBank NC_{—}001416; SEQ ID NO:2), in front of the operon or using one or more synthetic constitutively expressing promoters.

Accordingly, in one aspect, the activity of pyruvate dehydrogenase is modulated by increasing the activity of one or more genes of the pyruvate dehydrogenase complex consisting of (a) pyruvate dehydrogenase (E1), (b) dihydrolipoyl transacetylase, and (c) dihydrolipoyl dehydrogenase. It is understood that any one, two or three of these genes can be manipulated for increasing activity of pyruvate dehydrogenase. In another aspect, the activity of the pyruvate dehydrogenase complex can be modulated by attenuating the activity of an endogenous pyruvate dehydrogenase complex repressor gene, further detailed below. The activity of an endogenous pyruvate dehydrogenase complex repressor can be attenuated by deletion of the endogenous pyruvate dehydrogenase complex repressor gene.

In some cases, one or more genes of the pyruvate dehydrogenase complex are endogenous genes. Another way to increase the activity of the pyruvate dehydrogenase complex is by introducing into the microorganism one or more heterologous nucleic acids encoding one or more polypeptides from the group consisting of (a) pyruvate dehydrogenase (E1), (b) dihydrolipoyl transacetylase, and (c) dihydrolipoyl dehydrogenase.

By using any of these methods, the recombinant microorganism can produce increased amounts of acetyl Co-A in comparison to microorganisms wherein the activity of pyruvate dehydrogenase is not modulated. Modulating the activity of pyruvate dehydrogenase can result in more carbon flux into the mevalonate dependent biosynthetic pathway in comparison to microorganisms that do not have modulated pyruvate dehydrogenase expression.

Combinations of Mutations

It is understood that for any of the enzymes and/or enzyme pathways described herein, molecular manipulations that modulate any combination (two, three, four, five or six) of the enzymes and/or enzyme pathways described herein is expressly contemplated. For ease of the recitation of the combinations, citrate synthase (gltA) is designated as A, phosphotransacetylase (ptaB) is designated as B, acetate kinase (ackA) is designated as C, lactate dehydrogenase (ldhA) is designated as D, malic enzyme (sfcA or maeB) is designated as E, and pyruvate decarboxylase (aceE, aceF, and/or lpdA) is designated as F. As discussed above, aceE, aceF, and/or lpdA enzymes of the pyruvate decarboxylase complex can be used singly, or two of three enzymes, or three of three enzymes for increasing pyruvate decarboxylase activity.

Accordingly, for combinations of any two of the enzymes A-F, non-limiting combinations that can be used are: AB, AC, AD, AE, AF, BC, BD, BE, BF, CD, CE, CF, DE, DF and EF. For combinations of any three of the enzymes A-F, non-limiting combinations that can be used are: ABC, ABD, ABE, ABF, BCD, BCE, BCF, CDE, CDF, DEF, ACD, ACE, ACF, ADE, ADF, AEF, BDE, BDF, BEF, and CEF. For combinations of any four of the enzymes A-F, non-limiting combinations that can be used are: ABCD, ABCE, ABCF, ABDE, ABDF, ABEF, BCDE, BCDF, CDEF, ACDE, ACDF, ACEF, BCEF, BDEF, and ADEF. For combinations of any five of the enzymes A-F, non-limiting combinations that can be used are: ABCDE, ABCDF, ABDEF, BCDEF, ACDEF, and ABCEF. In another aspect, all six enzyme combinations are used: ABCDEF.

Accordingly, the recombinant microorganism as described herein can achieve increased mevalonate production that is increased compared to microorganisms that are not grown under conditions of tri-carboxylic acid (TCA) cycle activity, wherein metabolic carbon flux in the recombinant microorganism is directed towards mevalonate production by modulating the activity of one or more enzymes from the group consisting of (a) citrate synthase, (b) phosphotransacetylase and/or acetate kinase, (c) lactate dehydrogenase, (d) malic enzyme, and (e) pyruvate decarboxylase complex.

Other Regulators and Factors for Increased Production

Other molecular manipulations can be used to increase the flow of carbon towards mevalonate production. One method is to reduce, decrease or eliminate the effects of negative regulators for pathways that feed into the mevalonate pathway. For example, in some cases, the genes aceEF-lpdA are in an operon, with a fourth gene upstream pdhR. pdhR is a negative regulator of the transcription of its operon. In the absence of pyruvate, it binds its target promoter and represses transcription. It also regulates ndh and cyoABCD in the same way (Ogasawara, H. et al. 2007. J. Bact. 189:5534-5541). In one aspect, deletion of pdhR regulator can improve the supply of pyruvate, and hence the production of mevalonate, isoprene, isoprenoid precursors, and isoprenoids.

In other aspects, the introduction of 6-phosphogluconolactonase (PGL) into microorganisms (such as various E. coli strains) which lack PGL can be used to improve production of mevalonate, isoprene, isoprenoid precursors, and isoprenoids. PGL may be introduced using chromosomal integration or extra-chromosomal vehicles, such as plasmids. In certain embodiments, PGL may be deleted from the genome of microorganisms (such as various E. coli strains) which express an endogenous PGL to improve production of mevalonate and/or isoprene.

In some aspects of the invention, the recombinant cells described in any of the compositions or methods described herein can further comprise one or more nucleic acids encoding a phosphoketolase polypeptide or a polypeptide having phosphoketolase activity. In some aspects, the phosphoketolase polypeptide is an endogenous polypeptide. In some aspects, the endogenous nucleic acid encoding a phosphoketolase polypeptide is operably linked to a constitutive promoter. In some aspects, the endogenous nucleic acid encoding a phosphoketolase polypeptide is operably linked to an inducible promoter. In some aspects, the endogenous nucleic acid encoding a phosphoketolase polypeptide is operably linked to a strong promoter. In some aspects, more than one endogenous nucleic acid encoding a phosphoketolase polypeptide is used (e.g, 2, 3, 4, or more copies of an endogenous nucleic acid encoding a phosphoketolase polypeptide). In a particular aspect, the cells are engineered to overexpress the endogenous phosphoketolase polypeptide relative to wild-type cells. In some aspects, the endogenous nucleic acid encoding a phosphoketolase polypeptide is operably linked to a weak promoter.

Phosphoketolase enzymes catalyze the conversion of xylulose 5-phosphate to glyceraldehyde 3-phosphate and acetyl phosphate and/or the conversion of fructose 6-phosphate to erythrose 4-phosphate and acetyl phosphate. In certain embodiments, the phosphoketolase enzyme is capable of catalyzing the conversion of xylulose 5-phosphate to glyceraldehyde 3-phosphate and acetyl phosphate. In other embodiments, the phosphoketolase enzyme is capable of catalyzing the conversion of fructose 6-phosphate to erythrose 4-phosphate and acetyl phosphate. Thus, without being bound by theory, the expression of phosphoketolase as set forth herein can result in an increase in the amount of acetyl phosphate produced from a carbohydrate source. This acetyl phosphate can be converted into acetyl-CoA which can then be utilized by the enzymatic activities of the MVA pathway to produces mevalonate, isoprenoid precursor molecules, isoprene and/or isoprenoids. Thus the amount of these compounds produced from a carbohydrate substrate may be increased. Alternatively, production of Acetyl-P and AcCoA can be increased without the increase being reflected in higher intracellular concentration. In certain embodiments, intracellular acetyl-P or acetyl-CoA concentrations will remain unchanged or even decrease, even though the phosphoketolase reaction is taking place.

Exemplary phosphoketolase nucleic acids include nucleic acids that encode a polypeptide, fragment of a polypeptide, peptide, or fusion polypeptide that has at least one activity of a phosphoketolase polypeptide. Exemplary phosphoketolase polypeptides and nucleic acids include naturally-occurring polypeptides and nucleic acids from any of the source organisms described herein as well as mutant polypeptides and nucleic acids derived from any of the source organisms described herein. In some aspects, the phosphoketolase nucleic acid is a heterologous nucleic acid encoding a phosphoketolase polypeptide.

Standard methods can be used to determine whether a polypeptide has phosphoketolase peptide activity by measuring the ability of the peptide to convert D-fructose 6-phosphate or D-xylulose 5-phosphate into acetyl-P. Acetyl-P can then be converted into ferryl acetyl hydroxamate, which can be detected spectrophotometrically (Meile et al., J. Bact. 183:2929-2936, 2001). Any polypeptide identified as having phosphoketolase peptide activity as described herein is suitable for use in the present invention.

In other aspects, exemplary phosphoketolase nucleic acids include, for example, a phosphoketolase isolated from Lactobacillus reuteri, Bifidobacterium longum, Ferrimonas balearica, Pedobactor saltans, Streptomyces griseus, and/or Nocardiopsis dassonvillei. Additional examples of phosphoketolase enzymes which can be used herein are described in U.S. Pat. No. 7,785,858, which is incorporated by reference herein.

### Recombinant Cells Capable of Increased Production of Isoprene

Isoprene (2-methyl-1,3-butadiene) is an important organic compound used in a wide array of applications. For instance, isoprene is employed as an intermediate or a starting material in the synthesis of numerous chemical compositions and polymers, including in the production of synthetic rubber. Isoprene is also an important biological material that is synthesized naturally by many plants and animals.

Isoprene is produced from DMAPP by the enzymatic action of isoprene synthase. Therefore, without being bound to theory, it is thought that increasing the cellular production of mevalonate in host cells by any of the compositions and methods described above will similarly result in the production of higher amounts of isoprene. Increasing the molar yield of mevalonate production from glucose translates into higher molar yields of isoprenoid precursors and isoprenoids, including isoprene, produced from glucose when combined with appropriate enzymatic activity levels of mevalonate kinase, phosphomevalonate kinase, diphosphomevalonate decarboxylase, isopentenyl diphosphate isomerase and other appropriate enzymes for isoprene and isoprenoid production.

Production of isoprene can be made by using any of the recombinant host cells described here where acetoacetyl-CoA synthase is used to make acetoacetyl-CoA for downstream use in the MVA pathway. The use of acetoacetyl-CoA synthase can increase mevalonate production, which in turn, can be used to produce isoprene. Any of the recombinant host cells expressing one or more copies of a heterologous nucleic acid encoding upper MVA pathway polypeptides including, but not limited to, a HMG-CoA reductase and HMG-CoA synthase (e.g., an mvaS polypeptide from L. grayi, E. faecium, E. gallinarum, E. casseliflavus and/or E. faecalis) capable of increased production of mevalonate described above can also be capable of increased production of isoprene. In some aspects, these cells further comprise one or more heterologous nucleic acids encoding polypeptides of the lower MVA pathway and a heterologous nucleic acid encoding an isoprene synthase polypeptide.

Compositions of recombinant cells as described herein are contemplated within the scope of the invention as well. It is understood that recombinant cells also encompass progeny cells as well.

Exemplary Cell Culture Media

As used herein, the terms “minimal medium” or “minimal media” refer to growth medium containing the minimum nutrients possible for cell growth, generally, but not always, without the presence of one or more amino acids (e.g., 1, 2, 3, 4, 5, 6, 7, 8, 9, 10, or more amino acids). Minimal medium typically contains: (1) a carbon source for host cell growth; (2) various salts, which can vary among host cell species and growing conditions; and (3) water. The carbon source can vary significantly, from simple sugars like glucose to more complex hydrolysates of other biomass, such as yeast extract, as discussed in more detail below. The salts generally provide essential elements such as magnesium, nitrogen, phosphorus, and sulfur to allow the cells to synthesize proteins and nucleic acids. Minimal medium can also be supplemented with selective agents, such as antibiotics, to select for the maintenance of certain plasmids and the like. For example, if a microorganism is resistant to a certain antibiotic, such as ampicillin or tetracycline, then that antibiotic can be added to the medium in order to prevent cells lacking the resistance from growing. Medium can be supplemented with other compounds as necessary to select for desired physiological or biochemical characteristics, such as particular amino acids and the like.

Any minimal medium formulation can be used to cultivate the host cells. Exemplary minimal medium formulations include, for example, M9 minimal medium and TM3 minimal medium. Each liter of M9 minimal medium contains (1) 200 ml sterile M9 salts (64 g Na₂HPO₄-7H₂O, 15 g KH₂PO₄, 2.5 g NaCl, and 5.0 g NH₄Cl per liter); (2) 2 ml of 1 M MgSO₄ (sterile); (3) 20 ml of 20% (w/v) glucose (or other carbon source); and (4) 100 μl of 1 M CaCl₂ (sterile). Each liter of TM3 minimal medium contains (1) 13.6 g K₂HPO₄; (2) 13.6 g KH₂PO₄; (3) 2 g MgSO₄*7H₂O; (4) 2 g Citric Acid Monohydrate; (5) 0.3 g Ferric Ammonium Citrate; (6) 3.2 g (NH₄)₂SO₄; (7) 0.2 g yeast extract; and (8) 1 ml of 1000× Trace Elements solution; pH is adjusted to ˜6.8 and the solution is filter sterilized. Each liter of 1000× Trace Elements contains: (1) 40 g Citric Acid Monohydrate; (2) 30 g MnSO₄*H₂O; (3) 10 g NaCl; (4) 1 g FeSO₄*7H₂O; (4) 1 g CoCl₂*6H₂O; (5) 1 g ZnSO₄*7H₂O; (6) 100 mg CuSO₄*5H₂O; (7) 100 mg H₃BO₃; and (8) 100 mg NaMoO₄*2H₂O; pH is adjusted to ˜3.0.

An additional exemplary minimal media includes (1) potassium phosphate K₂HPO₄, (2) Magnesium Sulfate MgSO₄*7H₂O, (3) citric acid monohydrate C₆H₈O₇*H₂O, (4) ferric ammonium citrate NH₄FeC₆H₅O₇, (5) yeast extract (from biospringer), (6) 1000× Modified Trace Metal Solution, (7) sulfuric acid 50% w/v, (8) foamblast 882 (Emerald Performance Materials), and (9) Macro Salts Solution 3.36 ml All of the components are added together and dissolved in deionized H₂O and then heat sterilized. Following cooling to room temperature, the pH is adjusted to 7.0 with ammonium hydroxide (28%) and q.s. to volume. Vitamin Solution and spectinomycin are added after sterilization and pH adjustment.

Any carbon source can be used to cultivate the host cells. The term “carbon source” refers to one or more carbon-containing compounds capable of being metabolized by a host cell or organism. For example, the cell medium used to cultivate the host cells can include any carbon source suitable for maintaining the viability or growing the host cells. In some aspects, the carbon source is a carbohydrate (such as monosaccharide, disaccharide, oligosaccharide, or polysaccharides), or invert sugar (e.g., enzymatically treated sucrose syrup).

In some aspects, the carbon source includes yeast extract or one or more components of yeast extract. In some aspects, the concentration of yeast extract is 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. In some aspects, the carbon source includes both yeast extract (or one or more components thereof) and another carbon source, such as glucose.

Exemplary monosaccharides include glucose and fructose; exemplary oligosaccharides include lactose and sucrose, and exemplary polysaccharides include starch and cellulose. Exemplary carbohydrates include C6 sugars (e.g., fructose, mannose, galactose, or glucose) and C5 sugars (e.g., xylose or arabinose).

Exemplary Cell Culture Conditions

Materials and methods suitable for the maintenance and growth of the recombinant cells of the invention are described infra, e.g., in the Examples section. Other materials and methods suitable for the maintenance and growth of cell cultures are well known in the art. Exemplary techniques can be found in International Publication No. WO 2009/076676, U.S. patent application Ser. No. 12/335,071 (U.S. Publ. No. 2009/0203102), WO 2010/003007, US Publ. No. 2010/0048964, WO 2009/132220, US Publ. No. 2010/0003716, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass. In some aspects, the cells are cultured in a culture medium under conditions permitting the expression of one or more HMG-CoA reductase, HMG-CoA synthase, isoprene synthase, DXP pathway (e.g., DXS), IDI, lower MVA pathway polypeptides, or PGL polypeptides encoded by a nucleic acid inserted into the host cells.

Standard cell culture conditions can be used to culture the cells (see, for example, WO 2004/033646 and references cited therein). In some aspects, cells are grown and maintained at an appropriate temperature, gas mixture, and pH (such as at about 20° C. to about 37° C., at about 6% to about 84% CO₂, and at a pH between about 5 to about 9). In some aspects, cells are grown at 35° C. in an appropriate cell medium. In some aspects, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the host cells. In addition, more specific cell culture conditions can be used to culture the cells. For example, in some embodiments, the bacterial cells (such as E. coli cells) express one or more heterologous nucleic acids encoding HMG-CoA reductase under the control of a strong promoter in a low to medium copy plasmid and are cultured at 34° C.

Standard culture conditions and modes of fermentation, such as batch, fed-batch, or continuous fermentation that can be used are described in International Publication No. WO 2009/076676, U.S. patent application Ser. No. 12/335,071 (U.S. Publ. No. 2009/0203102), WO 2010/003007, US Publ. No. 2010/0048964, WO 2009/132220, US Publ. No. 2010/0003716. Batch and Fed-Batch fermentations are common and well known in the art and examples can be found in Brock, Biotechnology: A Textbook of Industrial Microbiology, Second Edition (1989) Sinauer Associates, Inc.

In some aspects, the cells are cultured under limited glucose conditions. By “limited glucose conditions” is meant that the amount of glucose that is added is less than or about 105% (such as about 100%, 90%, 80%, 70%, 60%, 50%, 40%, 30%, 20%, or 10%) of the amount of glucose that is consumed by the cells. In particular aspects, the amount of glucose that is added to the culture medium is approximately the same as the amount of glucose that is consumed by the cells during a specific period of time. In some aspects, the rate of cell growth is controlled by limiting the amount of added glucose such that the cells grow at the rate that can be supported by the amount of glucose in the cell medium. In some aspects, glucose does not accumulate during the time the cells are cultured. In various aspects, the cells are cultured under limited glucose conditions for greater than or about 1, 2, 3, 5, 10, 15, 20, 25, 30, 35, 40, 50, 60, or 70 hours. In various aspects, the cells are cultured under limited glucose conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited glucose conditions can allow more favorable regulation of the cells.

In some aspects, the host cells are grown in batch culture. The host cells can also be grown in fed-batch culture or in continuous culture. Additionally, the host cells can be cultured in minimal medium, including, but not limited to, any of the minimal media described above. The minimal medium can be further supplemented with 1.0% (w/v) glucose, or any other six carbon sugar, or less. Specifically, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose. Additionally, the minimal medium can be supplemented 0.1% (w/v) or less yeast extract. Specifically, the minimal medium can be supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract.

Methods of Using the Recombinant Cells to Produce Isoprene

Also provided herein are methods of producing isoprene comprising culturing any of the recombinant microorganisms described herein. In one aspect, isoprene can be produced by culturing recombinant host cells (e.g., bacterial cells) comprising one or more nucleic acids encoding a polypeptide capable of synthesizing acetoacetyl-CoA from malonyl-CoA and acetyl-CoA (e.g., acetoacetyl-CoA synthase) and one or more nucleic acids encoding: (a) an isoprene synthase polypeptide, wherein the isoprene synthase polypeptide is encoded by a heterologous nucleic acid; and (b) one or more mevalonate (MVA) pathway polypeptides. In one aspect, one or more heterologous nucleic acids encoding a HMG-CoA reductase, a lower MVA pathway polypeptide, and an isoprene synthase polypeptide can be used. In another aspect, isoprene can be produced by culturing recombinant host cells (e.g., bacterial cells) comprising one or more heterologous nucleic acids encoding a HMG-CoA reductase and HMG-CoA synthase, a lower MVA pathway polypeptide, and an isoprene synthase polypeptide. The isoprene can be produced from any of the cells described herein and according to any of the methods described herein. Any of the cells can be used for the purpose of producing isoprene from carbohydrates, including six carbon sugars such as glucose.

The cells can further comprise one or more nucleic acid molecules encoding the lower MVA pathway polypeptide(s) described above (e.g., MVK, PMK, MVD, and/or IDI) and any of the isoprene synthase polypeptide(s) described above (e.g. P. alba isoprene synthase). In some aspects, the host cells can be any of the cells described herein. Any of the isoprene synthases or variants thereof described herein, any of the host cell strains (e.g., bacterial strains) described herein, any of the promoters described herein, and/or any of the vectors described herein can also be used to produce isoprene using any of the energy sources (e.g. glucose or any other six carbon sugar) described herein. In some aspects, the method of producing isoprene further comprises a step of recovering the isoprene.

In some aspects, the amount of isoprene produced is measured at a productivity time point. In some aspects, the productivity for the cells is about any of the amounts of isoprene disclosed herein. In some aspects, the cumulative, total amount of isoprene produced is measured. In some aspects, the cumulative total productivity for the cells is about any of the amounts of isoprene disclosed herein.

In some aspects, any of the cells described herein (for examples the cells in culture) produce isoprene at greater than about any of or about any of 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, or more nmole of isoprene/gram of cells for the wet weight of the cells/hour (nmole/g_{wcm}/hr). In some aspects, the amount of isoprene is between about 2 to about 5,000 nmole/g_{wcm}/hr, such as between about 2 to about 100 nmole/g_{wcm}/hr, about 100 to about 500 nmole/g_{wcm}/hr, about 150 to about 500 nmole/g_{wcm}/hr, about 500 to about 1,000 nmole/g_{wcm}/hr, about 1,000 to about 2,000 nmole/g_{wcm}/hr, or about 2,000 to about 5,000 nmole/g_{wcm}/hr. In some aspects, the amount of isoprene is between about 20 to about 5,000 nmole/g_{wcm}/hr, about 100 to about 5,000 nmole/g_{wcm}/hr, about 200 to about 2,000 nmole/g_{wcm}/hr, about 200 to about 1,000 nmole/g_{wcm}/hr, about 300 to about 1,000 nmole/g_{wcm}/hr, or about 400 to about 1,000 nmole/g_{wcm}/hr.

In some aspects, the cells in culture produce isoprene at greater than or about 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, 10,000, 100,000, or more ng of isoprene/gram of cells for the wet weight of the cells/hr (ng/g_{wcm}/h). In some aspects, the amount of isoprene is between about 2 to about 5,000 ng/g_{wcm}/h, such as between about 2 to about 100 ng/g_{wcm}/h, about 100 to about 500 ng/g_{wcm}/h, about 500 to about 1,000 ng/g_{wcm}/h, about 1,000 to about 2,000 ng/g_{wcm}/h, or about 2,000 to about 5,000 ng/g_{wcm}/h. In some aspects, the amount of isoprene is between about 20 to about 5,000 ng/g_{wcm}/h, about 100 to about 5,000 ng/g_{wcm}/h, about 200 to about 2,000 ng/g_{wcm}/h, about 200 to about 1,000 ng/g_{wcm}/h, about 300 to about 1,000 ng/g_{wcm}/h, or about 400 to about 1,000 ng/g_{wcm}/h.

In some aspects, the cells in culture produce a cumulative titer (total amount) of isoprene at greater than about any of or about any of 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, 10,000, 50,000, 100,000, or more mg of isoprene/L of broth (mg/L_{broth}, wherein the volume of broth includes the volume of the cells and the cell medium). In some aspects, the amount of isoprene is between about 2 to about 5,000 mg/L_{broth}, such as between about 2 to about 100 mg/L_{broth}, about 100 to about 500 mg/L_{broth}, about 500 to about 1,000 mg/L_{broth}, about 1,000 to about 2,000 mg/L_{broth}, or about 2,000 to about 5,000 mg/L_{broth}. In some aspects, the amount of isoprene is between about 20 to about 5,000 mg/L_{broth}, about 100 to about 5,000 mg/L_{broth}, about 200 to about 2,000 mg/L_{broth}, about 200 to about 1,000 mg/L_{broth}, about 300 to about 1,000 mg/L_{broth}, or about 400 to about 1,000 mg/L_{broth}.

In some aspects, the isoprene produced by the cells in culture comprises at least about 1, 2, 5, 10, 15, 20, or 25% by volume of the fermentation offgas. In some aspects, the isoprene comprises between about 1 to about 25% by volume of the offgas, such as between about 5 to about 15%, about 15 to about 25%, about 10 to about 20%, or about 1 to about 10%.

Recombinant Cells Capable of Increased Production of Isoprenoid Precursors and/or Isoprenoids

Isoprenoids can be produced in many organisms from the synthesis of the isoprenoid precursor molecules which are the end products of the MVA pathway. As stated above, isoprenoids represent an important class of compounds and include, for example, food and feed supplements, flavor and odor compounds, and anticancer, antimalarial, antifungal, and antibacterial compounds.

As a class of molecules, isoprenoids are classified based on the number of isoprene units comprised in the compound. Monoterpenes comprise ten carbons or two isoprene units, sesquiterpenes comprise 15 carbons or three isoprene units, diterpenes comprise 20 carbons or four isoprene units, sesterterpenes comprise 25 carbons or five isoprene units, and so forth. Steroids (generally comprising about 27 carbons) are the products of cleaved or rearranged isoprenoids.

Isoprenoids can be produced from the isoprenoid precursor molecules IPP and DMAPP. These diverse compounds are derived from these rather simple universal precursors and are synthesized by groups of conserved polyprenyl pyrophosphate synthases (Hsieh et al., Plant Physiol. 2011 March; 155(3):1079-90). The various chain lengths of these linear prenyl pyrophosphates, reflecting their distinctive physiological functions, in general are determined by the highly developed active sites of polyprenyl pyrophosphate synthases via condensation reactions of allylic substrates (dimethylallyl diphosphate (C₅-DMAPP), geranyl pyrophosphate (C₁₀-GPP), farnesyl pyrophosphate (C₁₅-FPP), geranylgeranyl pyrophosphate (C₂₀-GGPP)) with corresponding number of isopentenyl pyrophosphates (C₅-IPP) (Hsieh et al., Plant Physiol. 2011 March; 155(3): 1079-90).

Production of isoprenoid precursors and/or isoprenoid can be made by using any of the recombinant host cells that comprise acetoacetyl-CoA synthase. In addition, these cells can express one or more copies of a heterologous nucleic acid encoding a HMG-CoA reductase and HMG-CoA synthase for increased production of mevalonate, isoprene, isoprenoid precursors and/or isoprenoids. Any of the recombinant host cells expressing one or more copies of a heterologous nucleic acid encoding a HMG-CoA reductase and HMG-CoA synthase capable of increased production of mevalonate or isoprene described above can also be capable of increased production of isoprenoid precursors and/or isoprenoids. In some aspects, these cells further comprise one or more heterologous nucleic acids encoding polypeptides of the lower MVA pathway, IDI, and/or the DXP pathway, as described above, and a heterologous nucleic acid encoding a polyprenyl pyrophosphate synthase polypeptide. Without being bound to theory, it is thought that increasing the cellular production of mevalonate in host cells by any of the compositions and methods described above will similarly result in the production of higher amounts of isoprenoid precursor molecules and/or isoprenoids. Increasing the molar yield of mevalonate production from glucose translates into higher molar yields of isoprenoid precursor molecules and/or isoprenoids, including isoprene, produced from glucose when combined with appropriate enzymatic activity levels of mevalonate kinase, phosphomevalonate kinase, diphosphomevalonate decarboxylase, isopentenyl diphosphate isomerase and other appropriate enzymes for isoprene and isoprenoid production.

Types of Isoprenoids

The recombinant microorganisms of the present invention are capable of increased production of isoprenoids and the isoprenoid precursor molecules DMAPP and IPP. Examples of isoprenoids include, without limitation, hemiterpenoids, monoterpenoids, sesquiterpenoids, diterpenoids, sesterterpenoids, triterpenoids, tetraterpenoids, and higher polyterpenoids. In some aspects, the hemiterpenoid is prenol (i.e., 3-methyl-2-buten-1-ol), isoprenol (i.e., 3-methyl-3-buten-1-ol), 2-methyl-3-buten-2-ol, or isovaleric acid. In some aspects, the monoterpenoid can be, without limitation, geranyl pyrophosphate, eucalyptol, limonene, or pinene. In some aspects, the sesquiterpenoid is farnesyl pyrophosphate, artemisinin, or bisabolol. In some aspects, the diterpenoid can be, without limitation, geranylgeranyl pyrophosphate, retinol, retinal, phytol, taxol, forskolin, or aphidicolin. In some aspects, the triterpenoid can be, without limitation, squalene or lanosterol. The isoprenoid can also be selected from the group consisting of abietadiene, amorphadiene, carene, farnesene, α-farnesene, β-farnesene, farnesol, geraniol, geranylgeraniol, linalool, limonene, myrcene, nerolidol, ocimene, patchoulol, β-pinene, sabinene, γ-terpinene, terpindene and valencene.

In some aspects, the tetraterpenoid is lycopene or carotene (a carotenoid). As used herein, the term “carotenoid” refers to a group of naturally-occurring organic pigments produced in the chloroplasts and chromoplasts of plants, of some other photosynthetic organisms, such as algae, in some types of fungus, and in some bacteria. Carotenoids include the oxygen-containing xanthophylls and the non-oxygen-containing carotenes. In some aspects, the carotenoids are selected from the group consisting of xanthophylls and carotenes. In some aspects, the xanthophyll is lutein or zeaxanthin. In some aspects, the carotenoid is α-carotene, β-carotene, γ-carotene, β-cryptoxanthin or lycopene.

Heterologous Nucleic Acids Encoding Polyprenyl Pyrophosphate Synthases Polypeptides

In some aspects of the invention, the recombinant cells described in any of the compositions or methods herein comprising acetoacetyl-CoA synthase further comprise one or more nucleic acids encoding a non-thiolase MVA pathway polypeptide(s), as described above, as well as one or more nucleic acids encoding a polyprenyl pyrophosphate synthase polypeptides(s). The polyprenyl pyrophosphate synthase polypeptide can be an endogenous polypeptide. The endogenous nucleic acid encoding a polyprenyl pyrophosphate synthase polypeptide can be operably linked to a constitutive promoter or can similarly be operably linked to an inducible promoter. The endogenous nucleic acid encoding a polyprenyl pyrophosphate synthase polypeptide can additionally be operably linked to a strong promoter. Alternatively, the endogenous nucleic acid encoding a polyprenyl pyrophosphate synthase polypeptide can be operably linked to a weak promoter. In particular, the cells can be engineered to over-express the endogenous polyprenyl pyrophosphate synthase polypeptide relative to wild-type cells.

In some aspects, the polyprenyl pyrophosphate synthase polypeptide is a heterologous polypeptide. The cells of the present invention can comprise more than one copy of a heterologous nucleic acid encoding a polyprenyl pyrophosphate synthase polypeptide. In some aspects, the heterologous nucleic acid encoding a polyprenyl pyrophosphate synthase polypeptide is operably linked to a constitutive promoter. In some aspects, the heterologous nucleic acid encoding a polyprenyl pyrophosphate synthase polypeptide is operably linked to an inducible promoter. In some aspects, the heterologous nucleic acid encoding a polyprenyl pyrophosphate synthase polypeptide is operably linked to a strong promoter. In some aspects, the heterologous nucleic acid encoding a polyprenyl pyrophosphate synthase polypeptide is operably linked to a weak promoter.

The nucleic acids encoding a polyprenyl pyrophosphate synthase polypeptide(s) can be integrated into a genome of the host cells or can be stably expressed in the cells. The nucleic acids encoding a polyprenyl pyrophosphate synthase polypeptide(s) can additionally be on a vector.

Exemplary polyprenyl pyrophosphate synthase nucleic acids include nucleic acids that encode a polypeptide, fragment of a polypeptide, peptide, or fusion polypeptide that has at least one activity of a polyprenyl pyrophosphate synthase. Polyprenyl pyrophosphate synthase polypeptides convert isoprenoid precursor molecules into more complex isoprenoid compounds. Exemplary polyprenyl pyrophosphate synthase polypeptides include polypeptides, fragments of polypeptides, peptides, and fusions polypeptides that have at least one activity of an isoprene synthase polypeptide. Exemplary polyprenyl pyrophosphate synthase polypeptides and nucleic acids include naturally-occurring polypeptides and nucleic acids from any of the source organisms described herein. In addition, variants of polyprenyl pyrophosphate synthase can possess improved activity such as improved enzymatic activity. In some aspects, a polyprenyl pyrophosphate synthase variant has other improved properties, such as improved stability (e.g., thermo-stability), and/or improved solubility. Exemplary polyprenyl pyrophosphate synthase nucleic acids can include nucleic acids which encode polyprenyl pyrophosphate synthase polypeptides such as, without limitation, geranyl diphosphosphate (GPP) synthase, farnesyl pyrophosphate (FPP) synthase, and geranylgeranyl pyrophosphate (GGPP) synthase, or any other known polyprenyl pyrophosphate synthase polypeptide.

In some aspects of the invention, the cells described in any of the compositions or methods herein further comprise one or more nucleic acids encoding a farnesyl pyrophosphate (FPP) synthase. The FPP synthase polypeptide can be an endogenous polypeptide encoded by an endogenous gene. In some aspects, the FPP synthase polypeptide is encoded by an endogenous ispA gene in E. coli. The endogenous nucleic acid encoding an FPP synthase polypeptide can be operably linked to a constitutive promoter or can similarly be operably linked to an inducible promoter. The endogenous nucleic acid encoding an FPP synthase polypeptide can additionally be operably linked to a strong promoter. In particular, the cells can be engineered to over-express the endogenous FPP synthase polypeptide relative to wild-type cells.

In some aspects, the FPP synthase polypeptide is a heterologous polypeptide. The cells of the present invention can comprise more than one copy of a heterologous nucleic acid encoding a FPP synthase polypeptide. In some aspects, the heterologous nucleic acid encoding a FPP synthase polypeptide is operably linked to a constitutive promoter. In some aspects, the heterologous nucleic acid encoding a FPP synthase polypeptide is operably linked to an inducible promoter. In some aspects, the heterologous nucleic acid encoding a polyprenyl pyrophosphate synthase polypeptide is operably linked to a strong promoter.

The nucleic acids encoding an FPP synthase polypeptide can be integrated into a genome of the host cells or can be stably expressed in the cells. The nucleic acids encoding an FPP synthase can additionally be on a vector.

Standard methods can be used to determine whether a polypeptide has polyprenyl pyrophosphate synthase polypeptide activity by measuring the ability of the polypeptide to convert IPP into higher order isoprenoids in vitro, in a cell extract, or in vivo. These methods are well known in the art and are described, for example, in U.S. Pat. No. 7,915,026; Hsieh et al., Plant Physiol. 2011 March; 155(3):1079-90; Danner et al., Phytochemistry. 2011 Apr. 12 [Epub ahead of print]; Jones et al., J Biol Chem. 2011 Mar. 24 [Epub ahead of print]; Keeling et al., BMC Plant Biol. 2011 Mar. 7; 11:43; Martin et al., BMC Plant Biol. 2010 Oct. 21; 10:226; Kumeta & Ito, Plant Physiol. 2010 December; 154(4):1998-2007; and Köllner & Boland, J Org Chem. 2010 Aug. 20; 75(16):5590-600.

Methods of Using the Recombinant Cells to Produce Isoprenoids and/or Isoprenoid Precursor Molecules

Also provided herein are methods of producing isoprenoid precursor molecules and/or isoprenoids comprising culturing recombinant microorganisms (e.g., recombinant bacterial cells) that comprise acetoacetyl-CoA synthase, a polyprenyl pyrophosphate synthase polypeptide, and one or more nucleic acids encoding a MVA pathway polypeptide including, but not limited to, HMG-CoA reductase, 3-hydroxy-3-methylglutaryl-CoA synthase (HMG-CoA synthase) polypeptides, 3-hydroxy-3-methylglutaryl-CoA reductase (HMG-CoA reductase) polypeptides, mevalonate kinase (MVK) polypeptides, phosphomevalonate kinase (PMK) polypeptides, diphosphomevalonte decarboxylase (MVD) polypeptides, phosphomevalonate decarboxylase (PMDC) polypeptides, isopentenyl phosphate kinase (IPK) polypeptides, IPP isomerase polypeptides, IDI polypeptides, and polypeptides (e.g., fusion polypeptides) having an activity of two or more MVA pathway polypeptides.

The isoprenoid precursor molecules and/or isoprenoids can be produced from any of the cells described herein and according to any of the methods described herein. Any of the cells can be used for the purpose of producing isoprenoid precursor molecules and/or isoprenoids from carbohydrates, including six carbon sugars such as glucose.

Thus, provided herein are methods of making isoprenoid precursor molecules and/or isoprenoids comprising culturing recombinant host cells comprising acetoacetyl-CoA synthase, a polyprenyl pyrophosphate synthase polypeptide, and one or more heterologous nucleic acids encoding a HMG-CoA reductase and HMG-CoA synthase, in a suitable condition for producing isoprene and producing isoprenoid precursor molecules and/or isoprenoids. The cells can further comprise one or more nucleic acid molecules encoding the lower MVA pathway polypeptide(s) described above (e.g., MVK, PMK, MVD, and/or IDI) and any of the polyprenyl pyrophosphate synthase polypeptide(s) described above. In some aspects, the host cells can be any of the cells described herein. Any of the polyprenyl pyrophosphate synthase or variants thereof described herein, any of the host strains described herein, any of the promoters described herein, and/or any of the vectors described herein can also be used to produce isoprenoid precursor molecules and/or isoprenoids using any of the energy sources (e.g. glucose or any other six carbon sugar) described herein. In some aspects, the method of producing isoprenoid precursor molecules and/or isoprenoids further comprises a step of recovering the isoprenoid precursor molecules and/or isoprenoids.

The method of producing isoprenoid precursor molecules and/or isoprenoids can similarly comprise the steps of: (a) culturing host cells (e.g., bacterial cells including, but not limited to, E. coli cells) that do not endogenously have a HMG-CoA reductase and HMG-CoA synthase, wherein the host cells heterologously express one or more copies of a gene encoding a HMG-CoA reductase and HMG-CoA synthase; and (b) producing isoprenoid precursor molecules and/or isoprenoids, wherein the host cells produce greater amounts of isoprenoid precursors and/or isoprenoids when compared to isoprenoids and/or isoprenoid precursor-producing host cells that do not comprise the HMG-CoA reductase and HMG-CoA synthase. In certain embodiment, the host cell is a bacterial cell, an algal cell, a fungal cell (including filamentous fungi), or a yeast cell.

The instant methods for the production of isoprenoid precursor molecules and/or isoprenoids can produce at least 5% greater amounts of isoprenoid precursors and/or isoprenoids when compared to isoprenoids and/or isoprenoid precursor-producing host cells that do not comprise the HMG-CoA reductase and HMG-CoA synthase and which have not been engineered for increased carbon flux to mevalonate production. Alternatively, the host cells can produce greater than about 1%, 2%, 3%, 4%, 5%, 6%, 7%, 8%, 9%, 10%, 11%, 12%, 13%, 14%, or 15% of isoprenoid precursors and/or isoprenoids, inclusive. In some aspects, the method of producing isoprenoid precursor molecules and/or isoprenoids further comprises a step of recovering the isoprenoid precursor molecules and/or isoprenoids.

Provided herein are methods of using any of the cells described above for enhanced isoprenoid and/or isoprenoid precursor molecule production. The production of isoprenoid precursor molecules and/or isoprenoids by the cells can be enhanced by the expression of acetoacetyl-CoA synthase and one or more heterologous nucleic acids encoding HMG-CoA reductase and HMG-CoA synthase, one or more heterologous nucleic acids encoding a lower MVA pathway polypeptide, and one or more heterologous nucleic acids encoding a polyprenyl pyrophosphate synthase polypeptide. As used herein, “enhanced” isoprenoid precursor and/or isoprenoid production refers to an increased cell productivity index (CPI) for isoprenoid precursor and/or isoprenoid production, an increased titer of isoprenoid precursors and/or isoprenoids, an increased mass yield of isoprenoid precursors and/or isoprenoids, and/or an increased specific productivity of isoprenoid precursors and/or isoprenoids by the cells described by any of the compositions and methods described herein compared to cells which do not have one or more heterologous nucleic acids encoding a polyprenyl pyrophosphate synthase polypeptide, a lower MVA pathway polypeptide(s), a DXP pathway polypeptide(s), and/or the HMG-CoA reductase and HMG-CoA synthase and which have not been engineered for increased carbon flux to mevalonate production. The production of isoprenoid precursor molecules and/or isoprenoids can be enhanced by about 5% to about 1,000,000 folds. The production of isoprenoid precursor molecules and/or isoprenoids can be enhanced by about 10% to about 1,000,000 folds (e.g., about 1 to about 500,000 folds, about 1 to about 50,000 folds, about 1 to about 5,000 folds, about 1 to about 1,000 folds, about 1 to about 500 folds, about 1 to about 100 folds, about 1 to about 50 folds, about 5 to about 100,000 folds, about 5 to about 10,000 folds, about 5 to about 1,000 folds, about 5 to about 500 folds, about 5 to about 100 folds, about 10 to about 50,000 folds, about 50 to about 10,000 folds, about 100 to about 5,000 folds, about 200 to about 1,000 folds, about 50 to about 500 folds, or about 50 to about 200 folds) compared to the production of isoprenoid precursor molecules and/or isoprenoids by cells without the expression of one or more heterologous nucleic acids encoding HMG-CoA reductase and HMG-CoA synthase and which have not been engineered for increased carbon flux to mevalonate production.

The production of isoprenoid precursor molecules and/or isoprenoids can also enhanced by at least about any of 10%, 20%, 30%, 40%, 50%, 60%, 70%, 80%, 90%, 1 fold, 2 folds, 5 folds, 10 folds, 20 folds, 50 folds, 100 folds, 200 folds, 500 folds, 1000 folds, 2000 folds, 5000 folds, 10,000 folds, 20,000 folds, 50,000 folds, 100,000 folds, 200,000 folds, 500,000 folds, or 1,000,000 folds compared to the production of isoprenoid precursor molecules and/or isoprenoids by cells without the expression of one or more heterologous nucleic acids encoding HMG-CoA reductase and HMG-CoA synthase and which have not been engineered for increased carbon flux to mevalonate production.

In addition, more specific cell culture conditions can be used to culture the cells in the methods described herein. For example, in some aspects, the method for the production of isoprenoid precursor molecules and/or isoprenoids comprises the steps of (a) culturing host cells (including bacterial cells including, but not limited to, E. coli cells) that do not endogenously have a HMG-CoA reductase and HMG-CoA synthase at 34° C., wherein the host cells heterologously express one or more copies of a gene encoding a HMG-CoA reductase and HMG-CoA synthase on a low to medium copy plasmid and under the control of a strong promoter; and (b) producing mevalonate. In some aspects, the method of producing mevalonate further comprises a step of recovering the isoprenoid precursor molecules and/or isoprenoids. In certain embodiment, the host cell is a bacterial cell, an algal cell, a fungal cell (including filamentous fungi), or a yeast cell.

### Exemplary Purification Methods

In some aspects, any of the methods described herein further include a step of recovering the compounds produced. In some aspects, any of the methods described herein further include a step of recovering the isoprene. In some aspects, the isoprene is recovered by absorption stripping (See, e.g., U.S. application Ser. No. 12/969,440). In some aspects, any of the methods described herein further include a step of recovering the heterologous polypeptide. In some aspects, any of the methods described herein further include a step of recovering the terpenoid or carotenoid.

Suitable purification methods are described in more detail in U.S. Patent Application Publication US2010/0196977 A1.

The invention can be further understood by reference to the following examples, which are provided by way of illustration and are not meant to be limiting.

### EXAMPLES

### Example 1

### Construction of Plasmids Encoding the Upper MVA Pathway for the Production of MVA via Acetoacetyl-CoA Synthase (NphT7)

An expression plasmid was generated to encode the nphT7 gene, mvaS gene, and mvaR gene that express Acetoacetyl-CoA synthase, HMG-CoA synthase, and HMG-CoA reductase, respectively. Briefly, forward and reverse primers were synthesized to amplify the mvaS gene (MCM489 and MCM490), mvaR gene (MCM491 and MCM492), and nphT7 gene (MCM495 and MCM496) from synthetic genes encoding Streptomyces proteins (Table 1). The MCM485 forward primer and MCM486 reverse primer were used to amplify the expression vector. The DNA template for amplification of the vector is pMCM1225 (Table 2). The DNA template for amplification of mvaS and mvaR from Streptomyces is StrepCL190 (DNA2.0) which contains a synthetic operon encoding mvaS and mvaR, also encodes Acetyl-CoA acetyltransferase (atoB). The pMCM1187 template which includes a synthetic gene encoding a His-tagged NphT7 is used for amplification of the gene encoding NphT7 (Genbank BAJ10048).

TABLE 1
Primers used for construction of pMCM1320 and pMCM1321

| Primer | | |
|---|---|---|
| Name | Description | Primer Sequence |
| MCM485 | pMCM82 USER 2 (for) | AGCTGG/IDEOXYU/ACCATA/ideoxy |
| | | U/GGGAAT/IDEOXYU/C |
| | | (SEQ ID NO: 3) |
| MCM486 | pMCM82 USER 1 (rev) | ATTTAA/ideoxyU/CGATACA/ideoxyU/ |
| | | TAATA/ideoxyU/ATACCTC |
| | | (SEQ ID NO: 4) |
| MCM489 | StrepCL190_mvaS USER 3 | ATGAGCA/ideoxyU/TTCTA/ideoxyU/C |
| | (for) | GGTA/ideoxyU/CCATGATCTT |
| | | (SEQ ID NO: 5) |
| MCM490 | StrepCL190_mvaS USER 4 | AACGTGC/ideoxyU/TCATAGA/ideoxy |
| | (rev) | U/ACGT/ideoxyU/TATGGTCGTT |
| | | (SEQ ID NO: 6) |
| MCM491 | StrepCL190_mvaR USER 1 | ATAT/ideoxyU/AATGTA/ideoxyU/CGA |
| | (for) | TTAAA/ideoxyU/AAGGAGGAATAAA |
| | | CCATGACCGAAACTCATGCAATTGC |
| | | TG |
| | | (SEQ ID NO: 7) |
| MCM492 | StrepCL190_mvaR USER 3 | ATACCGA/ideoxyU/AGAAA/ideoxyU/ |
| | (rev) | GCTCA/ideoxyU/TGGTATATCCTCCT |
| | | AGTGCGTCTAGATTACGCACCAACT |
| | | TTCGCGGTTTTGTTATCACGTTC |
| | | (SEQ ID NO: 8) |
| MCM495 | StrepCL190_nphT7 USER 4 | AACG/ideoxyU/ATCTA/ideoxyU/GAAG |
| | (for) | CACGT/ideoxyU/AAAGATCTCGCACT |
| | | AGGAGGATATACCAATGACCGACG |
| | | TGCGCTTTCGGAT |
| | | (SEQ ID NO: 9) |
| MCM496 | StrepCL190_nphT7 USER 2 | AATTCCCA/ideoxyU/ATGG/ideoxyU/A |
| | (rev) | CCAGC/ideoxyU/GCAGTCACCATTCA |
| | | ATCAACGCGAAGG |
| | | (SEQ ID NO: 10) |

Note:
/ideoxyU/ indicates an internal deoxyuridine nucleotide in the primer (Bitinaite J et al., Curr. Protoc. Mol. Biol. 86: 3.21.1-3.21.16, 2009).

TABLE 2
DNA Templates for construction of pMCM1320 and pMCM1321

| Plasmid Name | Description | Genes of Interest |
|---|---|---|
| pMCM1225 | pCL-Ptrc-Upper_E. gallinarum | vector |
| Streptomyces | Strep CL190 Upper MVA | Streptomyces mvaS, |
| CL190 Upper | pTrcHis2A | mvaR |
| pMCM1187 | pET15b-NphT7 (GeneOracle | Streptomyces nphT7 |
| | GcMM134) | |

Templates were amplified according the manufacturer's protocol for Agilent PfuTurbo Cx Hotstart DNA Polymerase (cat #600410). Reactions contain 5 μL buffer, 1 μL each 10 μM primer, 1 μL template plasmid (50-200 μg/uL), 1 μL 10 mM dNTPs, 40 μL ddH2O, 1 μL PfuCx (Table 3). Reactions were subsequently cycled as follows: one cycle at 95° C. for 2 minutes, thirty heating and cooling cycles (95° C. for 30 seconds, 55° C. for 30 seconds, 72° C. for 5 minutes and 30 seconds), and one cycle at 72° C. for 10 minutes. Reactions were held overnight at 4° C.

TABLE 3
Polymerase Chain Reaction (PCR) Reactions

| Amplicon | Primers | Template |
|---|---|---|
| Vector | MCM485/MCM486 | pMCM1225 |
| mvaS | MCM489/MCM490 | Streptomyces CL190 Upper |
| mvaR | MCM491/MCM492 | Streptomyces CL190 Upper |
| nphT7 | MCM495/MCM496 | pMCM1187 |

Following the amplification by PCR, 10 μL of the PCR reaction are mixed with 1 μL USER Enzyme (New England Biolabs # M5505S) and 1 μL DpnI (Roche) and then incubated at 37° C. for 2 hours. To generate an expression plasmid encoding the upper MVA pathway including the mvaR, mvaS, and NphT7 genes, ligation reactions were assembled from 2 μL of each USER reaction plus 8 μL of Buffer 1 and 1 μL ligase from the Roche Rapid Ligation Kit (11635379001). Reactions proceeded at room temperature for 1 hour and were stored at −20° C. overnight. To recover the ligated plasmid, 3 μL of the ligation was used to transform chemically competent TOP10 cells (Invitrogen #C404003) according to manufacturer instructions. Following recovery in 250 μL LB for 1 hr at 37° C., transformants were selected on LB/spec50 plates at 37° C. overnight. Single colonies were cultured in 5 mL LB/spec50 and stored at −80° C. DNA was extracted from the isolated colonies and successful generation of constructs encoding the upper MVA pathway including mvaR, mvaS, and NphT7 was confirmed by DNA sequencing (Table 4). Plasmids pMCM1320 and pMCM1321 were isolated from these strains.

TABLE 4
Isolated Strains Containing Plasmids Encoding
the Upper MVA Pathway

| Strain | Plasmid Description | Plasmid Name |
|---|---|---|
| MCM1320 | pCL-Ptrc-mvaR-mvaS- | pMCM1310 |
| | nphT7_StrepCL190_clone3-1 | |
| MCM1321 | pCL-Ptrc-mvaR-mvaS- | pMCM1321 |
| | nphT7_StrepCL190_clone3-2 | |

### Example 2

### Construction of Plasmid Encoding Isoprene Synthase and MVK for the Production of Isoprene

An expression plasmid for isoprene synthase and mevalonate kinase (MVK) with a bla gene encoding beta-lactamase was generated. Briefly, the bla gene from pUC19 DNA (Invitrogen) was amplified with primers MCM694 and MCM695 (Table 5). The expression plasmid pDu65 was amplified, not including the cmR marker gene, with primers MCM696 and MCM697. Amplicons were fused using the Invitrogen GENEART® Seamless Cloning and Assembly Kit (#A13288) according to the manufacturer's protocol and the product was subsequently transformed into chemically competent MD09-314 cells. Fused plasmid was selected on LB/carb50 plates at 37° C. overnight. A single colony was picked, grown in 5 mL LB/carb50 at 37° C., and stored at −80° C.

TABLE 5
Primers for Construction of pMCM1623

| Primer | | |
|---|---|---|
| Name | Description | Primer Sequence |
| MCM694 | bla - pDu65 - assemble 1 | CGGTGAACGCTCTCCTGAGTAGCATGAGATTAT |
| | | CAAAAAGGATCTTCACC |
| | | (SEQ ID NO: 11) |
| MCM695 | bla - pDu65 - assemble 2 | GGGACAGCTGATAGAAACAGAAGCCAAATATGT |
| | | ATCCGCTCATGAGACAA |
| | | (SEQ ID NO: 12) |
| MCM696 | bla - pDu65 - assemble 3 | TTGTCTCATGAGCGGATACATATTTGGCTTCTGT |
| | | TTCTATCAGCTGTCCC |
| | | (SEQ ID NO: 13) |
| MCM697 | bla - pDu65 - assemble 4 | GGTGAAGATCCTTTTTGATAATCTCATGCTACTC |
| | | AGGAGAGCGTTCACCG |
| | | (SEQ ID NO: 14) |

### Example 3

### Construction of Thiolase Deficient E. coli strain CMP861

An acetyl-CoA acetyltransferase (atoB) deficient strain was generated. Briefly, a DNA fragment containing the atoB gene interrupted by a kanamycin marker was amplified by PCR using strain JW2218 from the Keio collection (Baba et al. 2006. Mol. Syst. Biol. 2: 2006.0008) as a template, and primers atoBrecF (5′-GCAATTCCCCTTCTACGCTGGG-3′(SEQ ID NO:15)) and atoBrecR (5′-CTCGACCTTCACGTTGTTACGCC-3′ (SEQ ID NO:16)). The polymerase Herculase II Fusion (Agilent, Santa Clara, Calif.) was used according to the manufacturer's instructions. The PCR product obtained was used in a Recombineering Reaction (Gene Bridges, Heidelberg, Germany) as recommended by the manufacturer to integrate the PCR product at the atoB locus in strain CMP451. CMP451 is CMP258 (See U.S. patent application Ser. No. 12/978,324) with two modifications. Briefly, the promoter in front of the citrate synthase gene (gltA) in CMP258 was replaced by GI1.2 (U.S. Pat. No. 7,371,558). Two wild-type promoters have been described for gltA (Wilde, R, and J. Guest. 1986. J. Gen. Microbiol. 132:3239-3251) and the synthetic promoter was inserted just after the −35 region of the distal promoter. A PCR product was obtained using primers UpgltACm-F (5′-TATTTAATTTTTAATCATCTAATTTGACAATCATTCAACAAAGTTGTTACAATTAACCCT CACTAAAGGGCGG-3′(SEQ ID NO:17)) and DngltA1.xgiCm-R (5′-TCAACAGCTGTATCCCCGTTGAGGGTGAGTTTTGCTTTTGTATCAGCCATATATTCCACC AGCTATTTGTTAGTGAATAAAAGTGGTTGAATTATTTGCTCAGGATGTGGCATHGTCAA GGGCTAATACGACTCACTATAGGGCTCG-3′(SEQ ID NO:18)), and FRT-gb2-Cm-FRT template DNA from Gene Bridges (Heidelberg, Germany) as a template. The PCR product was purified and used in a lambda red-mediated recombination as described by the manufacturer (Gene Bridges, Heidelberg, Germany). Several colonies were selected for further characterization. The promoter region was PCR-amplified using primers gltAPromSeqF (5′-GGCAGTATAGGCTGTTCACAAAATC-3′(SEQ ID NO:19)) and gltApromSeqR (5′-CTTGACCCAGCGTGCCTTTCAGC-3′(SEQ ID NO:20)) and, as a template, DNA extracted by resuspending a colony in 30 μL H2O, heating at 95° C. for 4 min, spinning down, and using 2 μL of that material as a template in a 50 μL reaction. After observing the sequencing results of the PCR products obtained, a colony harboring the promoter GI1.2 (U.S. Pat. No. 7,371,558) was saved for further use and named CMP141. Strain MD09-313 was built by transducing CMP258 (See U.S. patent application Ser. No. 12/978,324) with a P1 lysate from strain MCM521 (See U.S. patent application Ser. No. 12/978,324) and selecting for colonies on Luria-Bertani plates containing 20 μg/ml kanamycin. P1 lysates were prepared according to the method described in Ausubel, et al., Current Protocols in Molecular Biology, John Wiley and Sons, Inc. The kanamycin marker was removed using the protocol recommended by the manufacturer (Gene Bridges, Heidelberg, Germany) to form strain MD09-314. A P1 lysate was made from strain CMP141 and was used to transduce strain MD09-314 to form CMP440. The chloramphenicol marker was removed using the protocol recommended by the manufacturer (Gene Bridges, Heidelberg, Germany) to form strain CMP451.

To generate the CMP861 strain, CMP451 underwent a recombineering reaction with the atoB:FRT-Kan-FRT PCR product and colonies were selected on LB+20 μg/ml of kanamycin. A single colony was picked and this strain was named CMP856. The kanamycin marker was subsequently removed from CMP856 by FRT recombination (Datsenko and Wanner. 2000. PNAS 97:6640-5), using plasmid pCP20. Once the transformants were selected on LA+50 μg/ml carbenicillin at 30° C., two colonies were re-streaked on a LB plate and incubated at 42° C. A kanamycin-sensitive colony was selected from those plates and named CMP861. The mutation was verified using primers atoBrecR and atoBcheckF (5′-GCTTATATGCGTGCTATCAGCG-3′ (SEQ ID NO:21)).

### Example 4

### Construction of Strains Encoding Pathways for the Production of MVA Via Acetoacetyl-CoA Synthase (NphT7)

Strains MCM1331, MCM1681, MCM1684, MCM1685, and MCM1686 were constructed by electroporating the indicated plasmid into the indicated parent strain (Table 6). Parent cells were grown in 5 mL LB supplemented with the indicated antibiotic from freezer vial scraping at 37° C. with shaking at 250 rpm. When the cell density reached an OD 0.5-0.8, the culture was placed on ice until cold and a 3 mL sample of the culture was washed in iced double distilled H2O three times before resuspension in 200 μL iced double distilled H2O. A 100 μL cell suspension sample was mixed with 1 to 3 μL DNA in an eppendorf tube and then transferred to a 2 mm electroporation cuvette for electroporation at 25 uFD, 200 ohms, 2.5 kV, and immediately quenched with 500 μL LB. Cells were recovered with shaking at 37° C. for 1 hr and transformants were selected overnight on LB plates with the indicated antibiotics at 37° C. A single colony was picked into 5 mL LB+antibiotics, grown at 37° C. and stored in 16.5% glycerol at −80° C. Strain MCM1686 contains the pCL1920 empty plasmid and therefore does not express the upper MVA pathway and directs isoprene production via the DXP pathway.

TABLE 6
Engineered Strains Encoding Pathways for the Production of MVA

| Strain | Genotype | Parent | Plasmid | Antibiotics |
|---|---|---|---|---|
| MCM1331 | atoB (Keio) + pCL-Ptrc-mvaR-mvaS- | Keio atoB | pMCM1321 | Spec50 |
| | nphT7_StrepCL190_clone3-2 | | | |
| MCM1681 | HMB gi1.2-gltA atoB::FRT pACYC-pTrcAlba- | CMP861 | pMCM1623 | Carb50 |
| | mMVK CARB | | | |
| MCM1684 | HMB gi1.2-gltA atoB::FRT pACYC-pTrcAlba- | MCM1681 | pMCM1320 | Carb50 |
| | mMVK CARB + pCL-Ptrc-mvaR-mvaS- | | | spec50 |
| | nphT7_StrepCL190_clone3-1 | | | |
| MCM1685 | HMB gi1.2-gltA atoB::FRT pACYC-pTrcAlba- | MCM1681 | pMCM1321 | Carb50 |
| | mMVK CARB + pCL-Ptrc-mvaR-mvaS- | | | spec50 |
| | nphT7_StrepCL190_clone3-2 | | | |
| MCM1686 | HMB gi1.2-gltA atoB::FRT pACYC-pTrcAlba- | MCM1681 | pCL1920 | Carb50 |
| | mMVK CARB + pCL1920 | | vector | spec50 |

### Example 5

### Isoprene Production via Acetoacetyl-CoA Synthase (NphT7) Using Engineered Strains

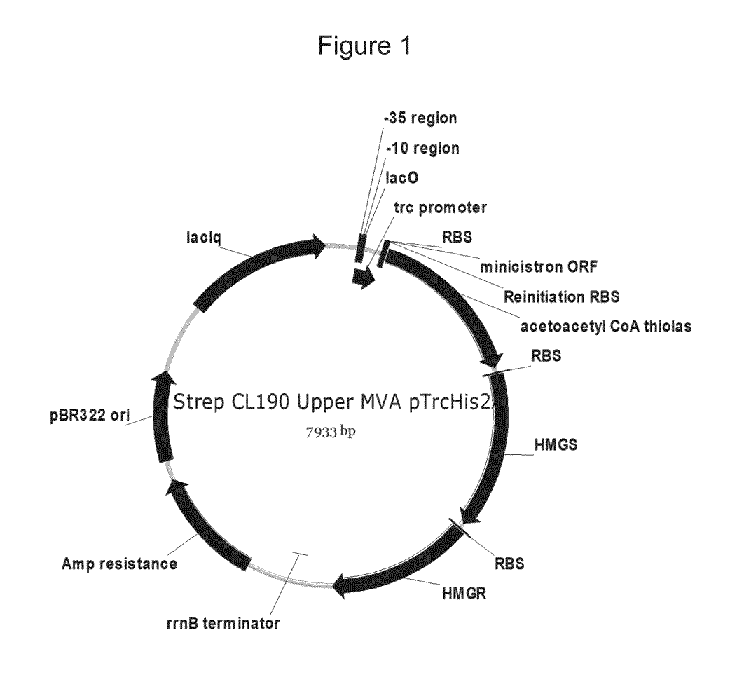

The specific productivity of isoprene from strains MCM1684, MCM1685, and MCM1686 was determined. A 10 uL sample of cell culture grown in LB media containing Carb50 and Spec 50 at a density near OD 1.0 was inoculated into TM3 cell culture medium containing 1% glucose, 0.02% yeast extract, carb50, and spec50 before culture overnight at 34° C. These cultures were used to inoculate 5 mL of the same TM3 media at OD 0.2 which was subsequently grown at 34° C. for 2 hours and 45 minutes with shaking at 250 rpm. Cultures were induced with 400 uM IPTG and grown for an additional 2 hours and 15 minutes. Culture density was determined from a 1:10 dilution of the broth. A 100 μL sample of the broth was incubated in a 2 mL headspace vial at 34° C. for 30 minutes, followed by heat kill at 70° C. for 12 minutes. Levels of isoprene in the headspace were determined by flame ionization detector coupled to a gas chromatograph (Model G1562A, Agilent Technologies) (Mergen et al., LC GC North America, 28(7):540-543, 2010). Data analysis of the isoprene produced demonstrated that MCM1684 and MCM1685, which contain the DXP pathway as well as a pathway for the production of isoprene via NphT7, makes isoprene at 2.4 times and 2.5 times the rate of MCM1686, respectively, which contains only the DXP pathway (Table 7 and FIG. 1).

TABLE 7
Isoprene Production

| | Isoprene Specific Productivity |
|---|---|
| Strain | (μg isoprene/L broth/OD/hr) |
| MCM1684 | 1286 |
| MCM1685 | 1381 |
| MCM1686 | 542 |

The following are sequences of various constructs made:

Strep CL190 Upper MVA pTrcHis2A
(SEQ ID NO: 22)
gtttgacagcttatcatcgactgcacggtgcaccaatgcttctggcgtcaggcagccatcggaagctgtggtatggctgtgcaggtcgtaaatcac
tgcataattcgtgtcgctcaaggcgcactcccgttctggataatgttttttgcgccgacatcataacggttctggcaaatattctgaaatgagctgttg
acaattaatcatccggctcgtataatgtgtggaattgtgagcggataacaatttcacacaggaaacagcgccgctgagaaaaagcgaagcggca
ctgctctttaacaatttatcagacaatctgtgtgggcactcgaccggaattatcgattaactttattattaaaaattaaagaggtatatattaatgtatcga
ttaaataaggaggaataaaccatggcatctgctactaactcatctgtaattgttgctggtgcacgtactccgatgggtcgcctgctgggttccctgaa
atctttctctggtgcggatctgggtggcttcgcgatcaaagcagcactggaccgcgcaggcattggcggcgaccaggtccagtacgttatcatg
ggtcaggtgttgcaggcaggtgcgggtcagattccggctcgtcaggcagcggttaaagctggcattccgatgaacgtaccagctctgaccatca
acaaagtgtgcctgtccggtctggacgcgatcgcgctggccgatcaactgatccgcgctggtgaatttgacattgttgtagcaggcggccagga
atctatgactaacgccccgcaccttctgccgaagtctcgtgaaggttataagtacggcgctatcgagatgctcgatgcaatggcttacgacggcct
gactgacccgtgggaaaacattccgatgggccagagcactgagaaacacaacacccgtctgggtatcggtcgtgaagttcaggatgagatcgc
ggcgctgtctcaccagcgtgctgctgcggcccagaaaaatggcctcttcgaagctgaaatcaccccgatcgaaattccgcagcgtaaaggtga
accggttgtattcagcaaagacgaaggcattcgcgcggaaaccactgctgaatccctcggcaaactgcgtccggctttcactaaggacggtact
atcactgcgggcaccgcgtctcagatttccgatggtgcggcagccgttgttgtcatgtcgaaagcgaaagcgttggaactgggtctggattggat
cgctgaaattggtgcccacggcaacgtggcaggcccggacaattctctgcaatcccaaccatcaaacgcaatcctgcacgcactgaagaaaga
aggtcttgaagtagaagacctggacctgatcgagatcaacgaagcgtttgccgcggttgcccaccaatctatgaaagacctgggtgtctccacc
gaaaaggttaacgtgaacggtggcgcaatcgccctgggccatccgattggcatgtccggtgctcgtctggttctgcacctggcactggagttga
agcgtcgtggtggtggcgtaggtgcagccgccctgtgtggtggcggtggtcagggcgatgcactgatcgtacgtgtcccgaaagcgtaagcct
tcttaaggtagctcagatacccttataagctttacaaggaggaaaaaaacatgagcatttctatcggtatccatgatctttcgttcgcgactaccgagt
tcgttctgcctcatacggctctggccgagtataatggtactgaaattggtaaatatcacgttggcatcggtcaacagagcatgagcgtacctgcgg
cggatgaggacatcgtgactatggccgcgaccgccgcacgtccgatcattgaacgcaacggtaaatcccgtatccgcactgtggtgttcgctac
ggagtcctccattgatcaagctaaagcgggtggcgtatacgtacattctctgctgggcctggaatccgcgtgccgtgtagttgaactgaaacagg
catgctacggtgcgacggcagcattgcagttcgctattggtctggttcgccgcgatccggcgcagcaggtgctggtgatcgcaagcgacgtttct
aaatacgagttggattcgccgggtgaggcaacccagggcgcagcggccgttgctatgctggttggcgcagatcctgcactgcttcgcattgaag
aaccgtccggcctcttcaccgcggacgttatggatttctggcgcccgaactacctgaccaccgccctggtagatggccaggagtccatcaacgc
ctacctgcaagcagttgaaggtgcgtggaaggactacgcagagcaggacggtcgtagccttgaagagtttgcagctttcgtttaccaccagccg
ttcactaaaatggcgtacaaagctcaccgccacctgctgaacttcaacggttacgacactgacaaagatgcgattgaaggcgccctgggtcaaa
ctaccgcttacaataatgtgatcggtaactcctacactgcgtcagtttatctgggcctggcggcgcttctggatcaggcggacgacctgaccggtc
gttctatcggcttcctgtcttacggctccggtagcgtagcggagtttttctcgggcaccgttgttgctggttaccgtgaacgtctgcgtactgaagcg
aaccaggaagctatcgcacgccgtaaatctgtcgattacgcgacttatcgtgaactgcatgagtacacgctcccgtctgatggtggtgatcacgc
cacgccggttcagaccaccggtccattccgtctggcaggtatcaacgaccataaacgtatctatgaagcacgttaagcctgacttaaggtagctg
ctttcgcccttatgagctctacaaggaggaaaaaaacatgaccgaaactcatgcaattgctggtgtaccgatgcgttgggttggcccgctgcgtat
ctctggcaacgtagcagaaaccgaaactcaggttccgctggcaacctacgaatctccgctgtggccgtctgtgggtcgtggtgcgaaggtatcc
cgtctgaccgaaaagggtatcgttgcaaccctcgttgacgaacgtatgacccgtagcgtcatcgtagaagccaccgacgcccagaccgcttata
tggctgcacagaccattcatgctcgcattgatgaactgcgcgaagtggttcgtggttgttcccgtttcgcgcagctgattaatatcaaacatgaaatt
aacgccaacttgctgttcatccgctttgaatttaccaccggtgacgcatctggccataacatggccaccctggcgtctgacgttctgttgggtcacc
tcctggaaaccatcccaggcatctcctacggctccatttctggcaactactgcactgacaagaaagcaactgccattaacggtatcctgggtcgc
ggcaaaaacgttatcaccgaattgctggtgccgcgcgatgttgtagaaaataacctgcacaccaccgctgcgaaaatcgttgaactgaacatcc
gtaaaaacctgctgggcactctcctggcaggcggcatccgttctgcgaacgcgcattttgcaaatatgctgctgggtttctacctggcaactggcc
aggacgcagcaaacatcgttgagggtagccagggcgttgtaatggcggaagatcgtgacggtgatctgtacttcgcgtgcacccttccgaatct
gatcgtgggtactgttggcaacggtaagggcctgggttttgttgaaacgaatctggcccgtctgggctgtcgcgcggatcgcgaaccgggcga
aaatgcgcgccgtctggccgttatcgccgctgccaccgtgctgtgtggtgaactgtccctgttggccgcgcagaccaaccctggtgaactgatg
cgtgcacatgttcagctggaacgtgataacaaaaccgcgaaagttggtgcgtaaagatctgcagctggtaccatatgggaattcgaagcttggg
cccgaacaaaaactcatctcagaagaggatctgaatagcgccgtcgaccatcatcatcatcatcattgagtttaaacggtctccagcttggctgtttt
ggcggatgagagaagattttcagcctgatacagattaaatcagaacgcagaagcggtctgataaaacagaatttgcctggcggcagtagcgcg
gtggtcccacctgaccccatgccgaactcagaagtgaaacgccgtagcgccgatggtagtgtggggtctccccatgcgagagtagggaactg
ccaggcatcaaataaaacgaaaggctcagtcgaaagactgggcctttcgttttatctgttgtttgtcggtgaacgctctcctgagtaggacaaatcc
gccgggagcggatttgaacgttgcgaagcaacggcccggagggtggcgggcaggacgcccgccataaactgccaggcatcaaattaagca
gaaggccatcctgacggatggcctttttgcgtttctacaaactctttttgtttatttttctaaatacattcaaatatgtatccgctcatgagacaataaccc
tgataaatgcttcaataatattgaaaaaggaagagtatgagtattcaacatttccgtgtcgcccttattcccttttttgcggcattttgccttcctgttttt
gctcacccagaaacgctggtgaaagtaaaagatgctgaagatcagttgggtgcacgagtgggttacatcgaactggatctcaacagcggtaagat
ccttgagagttttcgccccgaagaacgttttccaatgatgagcacttttaaagttctgctatgtggcgcggtattatcccgtgttgacgccgggcaag
agcaactcggtcgccgcatacactattctcagaatgacttggttgagtactcaccagtcacagaaaagcatcttacggatggcatgacagtaaga
gaattatgcagtgctgccataaccatgagtgataacactgcggccaacttacttctgacaacgatcggaggaccgaaggagctaaccgcttttttg
cacaacatgggggatcatgtaactcgccttgatcgttgggaaccggagctgaatgaagccataccaaacgacgagcgtgacaccacgatgcct
gtagcaatggcaacaacgttgcgcaaactattaactggcgaactacttactctagcttcccggcaacaattaatagactggatggaggcggataa
agttgcaggaccacttctgcgctcggcccttccggctggctggtttattgctgataaatctggagccggtgagcgtgggtctcgcggtatcattgc
agcactggggccagatggtaagccctcccgtatcgtagttatctacacgacggggagtcaggcaactatggatgaacgaaatagacagatcgc
tgagataggtgcctcactgattaagcattggtaactgtcagaccaagtttactcatatatactttagattgatttaaaacttcatttttaatttaaaaggat
ctaggtgaagatcctttttgataatctcatgaccaaaatcccttaacgtgagttttcgttccactgagcgtcagaccccgtagaaaagatcaaaggat
cttcttgagatcctttttttctgcgcgtaatctgctgcttgcaaacaaaaaaaccaccgctaccagcggtggtttgtttgccggatcaagagctacca
actctttttccgaaggtaactggcttcagcagagcgcagataccaaatactgtccttctagtgtagccgtagttaggccaccacttcaagaactctgt
agcaccgcctacatacctcgctctgctaatcctgttaccagtggctgctgccagtggcgataagtcgtgtcttaccgggttggactcaagacgata
gttaccggataaggcgcagcggtcgggctgaacggggggttcgtgcacacagcccagcttggagcgaacgacctacaccgaactgagatac
ctacagcgtgagctatgagaaagcgccacgcttcccgaagggagaaaggcggacaggtatccggtaagcggcagggtcggaacaggagag
cgcacgagggagcttccagggggaaacgcctggtatctttatagtcctgtcgggtttcgccacctctgacttgagcgtcgatttttgtgatgctcgt
caggggggcggagcctatggaaaaacgccagcaacgcggcctttttacggttcctggccttttgctggccttttgctcacatgttctttcctgcgtta
tcccctgattctgtggataaccgtattaccgcctttgagtgagctgataccgctcgccgcagccgaacgaccgagcgcagcgagtcagtgagcg
aggaagcggaagagcgcctgatgcggtattttctccttacgcatctgtgcggtatttcacaccgcatatggtgcactctcagtacaatctgctctgat
gccgcatagttaagccagtatacactccgctatcgctacgtgactgggtcatggctgcgccccgacacccgccaacacccgctgacgcgccct
gacgggcttgtctgctcccggcatccgcttacagacaagctgtgaccgtctccgggagctgcatgtgtcagaggttttcaccgtcatcaccgaaa
cgcgcgaggcagcagatcaattcgcgcgcgaaggcgaagcggcatgcatttacgttgacaccatcgaatggtgcaaaacctttcgcggtatgg
catgatagcgcccggaagagagtcaattcagggtggtgaatgtgaaaccagtaacgttatacgatgtcgcagagtatgccggtgtctcttatcag
accgtttcccgcgtggtgaaccaggccagccacgtttctgcgaaaacgcgggaaaaagtggaagcggcgatggcggagctgaattacattcc
caaccgcgtggcacaacaactggcgggcaaacagtcgttgctgattggcgttgccacctccagtctggccctgcacgcgccgtcgcaaattgtc
gcggcgattaaatctcgcgccgatcaactgggtgccagcgtggtggtgtcgatggtagaacgaagcggcgtcgaagcctgtaaagcggcggt
gcacaatcttctcgcgcaacgcgtcagtgggctgatcattaactatccgctggatgaccaggatgccattgctgtggaagctgcctgcactaatgt
tccggcgttatttcttgatgtctctgaccagacacccatcaacagtattattttctcccatgaagacggtacgcgactgggcgtggagcatctggtcg
cattgggtcaccagcaaatcgcgctgttagcgggcccattaagttctgtctcggcgcgtctgcgtctggctggctggcataaatatctcactcgca
atcaaattcagccgatagcggaacgggaaggcgactggagtgccatgtccggttttcaacaaaccatgcaaatgctgaatgagggcatcgttcc
cactgcgatgctggttgccaacgatcagatggcgctgggcgcaatgcgcgccattaccgagtccgggctgcgcgttggtgcggatatctcggt
agtgggatacgacgataccgaagacagctcatgttatatcccgccgtcaaccaccatcaaacaggattttcgcctgctggggcaaaccagcgtg
gaccgcttgctgcaactctctcagggccaggcggtgaagggcaatcagctgttgcccgtctcactggtgaaaagaaaaaccaccctggcgccc
aatacgcaaaccgcctctccccgcgcgttggccgattcattaatgcagctggcacgacaggtttcccgactggaaagcgggcagtgagcgcaa
cgcaattaatgtgagttagcgcgaattgatctg
pMCM1187 - pET15b-NphT7 (GeneOracle GcMM134)
(SEQ ID NO: 23)
ttctcatgtttgacagcttatcatcgataagctttaatgcggtagtttatcacagttaaattgctaacgcagtcaggcaccgtgtatgaaatctaacaat
gcgctcatcgtcatcctcggcaccgtcaccctggatgctgtaggcataggcttggttatgccggtactgccgggcctcttgcgggatatccggat
atagttcctcctttcagcaaaaaacccctcaagacccgtttagaggccccaaggggttatgctagttattgctcagcggtggcagcagccaactca
gcttcctttcgggctttgttagcagccggatccttatcaccattcaatcaacgcgaaggaagctgccatgcccccgccgaaacctgccaacagaa
cgagttcgcctgggcgaaaggacccagcgcgcactgctgcatccatagtgataggtatggacgcagcgcctgtattaccgtatgtttcaactgta
cggtgcatagtggcacgagggagatgcaattcgccgaacacttcatccagcataacgccgttcgcctggtgtgggacaaagtgcgaaatgtctg
ccgcatccactcctgcttcatgcagaaaccccttaatcagctgcggaaggtgctctgtcacgaaacgccgcacttcgcgaccatccattgcaaag
tattgaagaccggcatcaagcccgtctgtgtcaagaggttgccgtgaaccacctgctggcacgcggatcagatccgtcagtcctccgaaggtat
gaagagcgacccggcgcacgattggcccggttccagtggacgtcgggcctaataccatcgcaccagccccgtcaccaaaaagtaccactgttt
tacgatccgcagggttcaggatacgcgaatacaggtccgcccctattaccagagcgtatccgccgcggtaaaccaacgtacccgcaactgacg
aaagtgcgaaaaccgtcccgctgcacactgcgttcacatcgaatgcggcggtgcctgttgcacccaaatgatgctgaacataagcagcggtag
ggggctgaggacggtccggggtcgaagtagccaccgcaataacggttaactgctccggagttatgcccgcagccttcagtgccgcacgaccc
gcggcagtggcaaggtcactagtcgcttgatcatctgccgcccaccgtctctgacgtatacccgtcttgcgtgtaatccaatcatcgtccacacca
gccggtgcgccaacttcgtcattactcacgatgcgttccggcacgtacgcaccggtaccaattatccgaaagcgcacgtcggtcatatggctgcc
gcgcggcaccaggccgctgctgtgatgatgatgatgatggctgctgcccatggtatatctccttcttaaagttaaacaaaattatttctagagggga
attgttatccgctcacaattcccctatagtgagtcgtattaatttcgcgggatcgagatctcgatcctctacgccggacgcatcgtggccggcatca
ccggcgccacaggtgcggttgctggcgcctatatcgccgacatcaccgatggggaagatcgggctcgccacttcgggctcatgagcgcttgttt
cggcgtgggtatggtggcaggccccgtggccgggggactgttgggcgccatctccttgcatgcaccattccttgcggcggcggtgctcaacg
gcctcaacctactactgggctgcttcctaatgcaggagtcgcataagggagagcgtcgagatcccggacaccatcgaatggcgcaaaacctttc
gcggtatggcatgatagcgcccggaagagagtcaattcagggtggtgaatgtgaaaccagtaacgttatacgatgtcgcagagtatgccggtgt
ctcttatcagaccgtttcccgcgtggtgaaccaggccagccacgtttctgcgaaaacgcgggaaaaagtggaagcggcgatggcggagctga
attacattcccaaccgcgtggcacaacaactggcgggcaaacagtcgttgctgattggcgttgccacctccagtctggccctgcacgcgccgtc
gcaaattgtcgcggcgattaaatctcgcgccgatcaactgggtgccagcgtggtggtgtcgatggtagaacgaagcggcgtcgaagcctgtaa
agcggcggtgcacaatcttctcgcgcaacgcgtcagtgggctgatcattaactatccgctggatgaccaggatgccattgctgtggaagctgcct
gcactaatgttccggcgttatttcttgatgtctctgaccagacacccatcaacagtattattttctcccatgaagacggtacgcgactgggcgtggag
catctggtcgcattgggtcaccagcaaatcgcgctgttagcgggcccattaagttctgtctcggcgcgtctgcgtctggctggctggcataaatat
ctcactcgcaatcaaattcagccgatagcggaacgggaaggcgactggagtgccatgtccggttttcaacaaaccatgcaaatgctgaatgagg
gcatcgttcccactgcgatgctggttgccaacgatcagatggcgctgggcgcaatgcgcgccattaccgagtccgggctgcgcgttggtgcgg
atatctcggtagtgggatacgacgataccgaagacagctcatgttatatcccgccgttaaccaccatcaaacaggattttcgcctgctggggcaaa
ccagcgtggaccgcttgctgcaactctctcagggccaggcggtgaagggcaatcagctgttgcccgtctcactggtgaaaagaaaaaccaccc
tggcgcccaatacgcaaaccgcctctccccgcgcgttggccgattcattaatgcagctggcacgacaggtttcccgactggaaagcgggcagt
gagcgcaacgcaattaatgtaagttagctcactcattaggcaccgggatctcgaccgatgcccttgagagccttcaacccagtcagctccttccg
gtgggcgcggggcatgactatcgtcgccgcacttatgactgtcttctttatcatgcaactcgtaggacaggtgccggcagcgctctgggtcattttc
ggcgaggaccgctttcgctggagcgcgacgatgatcggcctgtcgcttgcggtattcggaatcttgcacgccctcgctcaagccttcgtcactgg
tcccgccaccaaacgtttcggcgagaagcaggccattatcgccggcatggcggccgacgcgctgggctacgtcttgctggcgttcgcgacgc
gaggctggatggccttccccattatgattcttctcgcttccggcggcatcgggatgcccgcgttgcaggccatgctgtccaggcaggtagatgac
gaccatcagggacagcttcaaggatcgctcgcggctcttaccagcctaacttcgatcactggaccgctgatcgtcacggcgatttatgccgcctc
ggcgagcacatggaacgggttggcatggattgtaggcgccgccctataccttgtctgcctccccgcgttgcgtcgcggtgcatggagccgggc
cacctcgacctgaatggaagccggcggcacctcgctaacggattcaccactccaagaattggagccaatcaattcttgcggagaactgtgaatg
cgcaaaccaacccttggcagaacatatccatcgcgtccgccatctccagcagccgcacgcggcgcatctcgggcagcgttgggtcctggcca
cgggtgcgcatgatcgtgctcctgtcgttgaggacccggctaggctggcggggttgccttactggttagcagaatgaatcaccgatacgcgagc
gaacgtgaagcgactgctgctgcaaaacgtctgcgacctgagcaacaacatgaatggtcttcggtttccgtgtttcgtaaagtctggaaacgcgg
aagtcagcgccctgcaccattatgttccggatctgcatcgcaggatgctgctggctaccctgtggaacacctacatctgtattaacgaagcgctgg
cattgaccctgagtgatttttctctggtcccgccgcatccataccgccagttgtttaccctcacaacgttccagtaaccgggcatgttcatcatcagta
acccgtatcgtgagcatcctctctcgtttcatcggtatcattacccccatgaacagaaatcccccttacacggaggcatcagtgaccaaacaggaa
aaaaccgcccttaacatggcccgctttatcagaagccagacattaacgcttctggagaaactcaacgagctggacgcggatgaacaggcagac
atctgtgaatcgcttcacgaccacgctgatgagctttaccgcagctgcctcgcgcgtttcggtgatgacggtgaaaacctctgacacatgcagctc
ccggagacggtcacagcttgtctgtaagcggatgccgggagcagacaagcccgtcagggcgcgtcagcgggtgttggcgggtgtcggggc
gcagccatgacccagtcacgtagcgatagcggagtgtatactggcttaactatgcggcatcagagcagattgtactgagagtgcaccatatatgc
ggtgtgaaataccgcacagatgcgtaaggagaaaataccgcatcaggcgctcttccgcttcctcgctcactgactcgctgcgctcggtcgttcgg
ctgcggcgagcggtatcagctcactcaaaggcggtaatacggttatccacagaatcaggggataacgcaggaaagaacatgtgagcaaaagg
ccagcaaaaggccaggaaccgtaaaaaggccgcgttgctggcgtttttccataggctccgcccccctgacgagcatcacaaaaatcgacgctc
aagtcagaggtggcgaaacccgacaggactataaagataccaggcgtttccccctggaagctccctcgtgcgctctcctgttccgaccctgccg
cttaccggatacctgtccgcctttctcccttcgggaagcgtggcgctttctcatagctcacgctgtaggtatctcagttcggtgtaggtcgttcgctcc
aagctgggctgtgtgcacgaaccccccgttcagcccgaccgctgcgccttatccggtaactatcgtcttgagtccaacccggtaagacacgactt
atcgccactggcagcagccactggtaacaggattagcagagcgaggtatgtaggcggtgctacagagttcttgaagtggtggcctaactacgg
ctacactagaaggacagtatttggtatctgcgctctgctgaagccagttaccttcggaaaaagagttggtagctcttgatccggcaaacaaaccac
cgctggtagcggtggtttttttgtttgcaagcagcagattacgcgcagaaaaaaaggatctcaagaagatcctttgatcttttctacggggtctgacg
ctcagtggaacgaaaactcacgttaagggattttggtcatgagattatcaaaaaggatcttcacctagatccttttaaattaaaaatgaagttttaaatc
aatctaaagtatatatgagtaaacttggtctgacagttaccaatgcttaatcagtgaggcacctatctcagcgatctgtctatttcgttcatccatagttg
cctgactccccgtcgtgtagataactacgatacgggagggcttaccatctggccccagtgctgcaatgataccgcgagacccacgctcaccgg
ctccagatttatcagcaataaaccagccagccggaagggccgagcgcagaagtggtcctgcaactttatccgcctccatccagtctattaattgtt
gccgggaagctagagtaagtagttcgccagttaatagtttgcgcaacgttgttgccattgctgcaggcatcgtggtgtcacgctcgtcgtttggtat
ggcttcattcagctccggttcccaacgatcaaggcgagttacatgatcccccatgttgtgcaaaaaagcggttagctccttcggtcctccgatcgtt
gtcagaagtaagttggccgcagtgttatcactcatggttatggcagcactgcataattctcttactgtcatgccatccgtaagatgcttttctgtgactg
gtgagtactcaaccaagtcattctgagaatagtgtatgcggcgaccgagttgctcttgcccggcgtcaacacgggataataccgcgccacatag
cagaactttaaaagtgctcatcattggaaaacgttcttcggggcgaaaactctcaaggatcttaccgctgttgagatccagttcgatgtaacccact
cgtgcacccaactgatcttcagcatcttttactttcaccagcgtttctgggtgagcaaaaacaggaaggcaaaatgccgcaaaaaagggaataag
ggcgacacggaaatgttgaatactcatactcttcctttttcaatattattgaagcatttatcagggttattgtctcatgagcggatacatatttgaatgtat
ttagaaaaataaacaaataggggttccgcgcacatttccccgaaaagtgccacctgacgtctaagaaaccattattatcatgacattaacctataaa
aataggcgtatcacgaggccctttcgtcttcaagaa
pMCM1320 and pMCM1321 - pCL-Ptrc-mvaR-mvaS -nphT7 StrepCL190
(SEQ ID NO: 24)
ctgcagctggtaccatatgggaattcgaagcttgggcccgaacaaaaactcatctcagaagaggatctgaatagcgccgtcgaccatcatcatc
atcatcattgagtttaaacggtctccagcttggctgttttggcggatgagagaagattttcagcctgatacagattaaatcagaacgcagaagcggt
ctgataaaacagaatttgcctggcggcagtagcgcggtggtcccacctgaccccatgccgaactcagaagtgaaacgccgtagcgccgatggt
agtgtggggtctccccatgcgagagtagggaactgccaggcatcaaataaaacgaaaggctcagtcgaaagactgggcctttcgttttatctgtt
gtttgtcggtgaacgctctcctgagtaggacaaatccgccgggagcggatttgaacgttgcgaagcaacggcccggagggtggcgggcagga
cgcccgccataaactgccaggcatcaaattaagcagaaggccatcctgacggatggcctttttgcgtttctacaaactctttttgtttatttttctaaat
acattcaaatatgtatccgctcatgagacaataaccctgataaatgcttcaataatctggcgtaatagcgaagaggcccgcaccgatcgcccttcc
caacagttgcgcagcctgaatggcgaatggcgcctgatgcggtattttctccttacgcatctgtgcggtatttcacaccgcatatggtgcactctca
gtacaatctgctctgatgccgcatagttaagccagccccgacacccgccaacacccgctgacgagcttagtaaagccctcgctagattttaatgc
ggatgttgcgattacttcgccaactattgcgataacaagaaaaagccagcctttcatgatatatctcccaatttgtgtagggcttattatgcacgctta
aaaataataaaagcagacttgacctgatagtttggctgtgagcaattatgtgcttagtgcatctaacgcttgagttaagccgcgccgcgaagcggc
gtcggcttgaacgaattgttagacattatttgccgactaccttggtgatctcgcctttcacgtagtggacaaattcttccaactgatctgcgcgcgag
gccaagcgatcttcttcttgtccaagataagcctgtctagcttcaagtatgacgggctgatactgggccggcaggcgctccattgcccagtcggc
agcgacatccttcggcgcgattttgccggttactgcgctgtaccaaatgcgggacaacgtaagcactacatttcgctcatcgccagcccagtcgg
gcggcgagttccatagcgttaaggtttcatttagcgcctcaaatagatcctgttcaggaaccggatcaaagagttcctccgccgctggacctacca
aggcaacgctatgttctcttgcttttgtcagcaagatagccagatcaatgtcgatcgtggctggctcgaagatacctgcaagaatgtcattgcgctg
ccattctccaaattgcagttcgcgcttagctggataacgccacggaatgatgtcgtcgtgcacaacaatggtgacttctacagcgcggagaatctc
gctctctccaggggaagccgaagtttccaaaaggtcgttgatcaaagctcgccgcgttgtttcatcaagccttacggtcaccgtaaccagcaaatc
aatatcactgtgtggcttcaggccgccatccactgcggagccgtacaaatgtacggccagcaacgtcggttcgagatggcgctcgatgacgcc
aactacctctgatagttgagtcgatacttcggcgatcaccgcttccctcatgatgtttaactttgttttagggcgactgccctgctgcgtaacatcgttg
ctgctccataacatcaaacatcgacccacggcgtaacgcgcttgctgcttggatgcccgaggcatagactgtaccccaaaaaaacagtcataac
aagccatgaaaaccgccactgcgccgttaccaccgctgcgttcggtcaaggttctggaccagttgcgtgagcgcatacgctacttgcattacagc
ttacgaaccgaacaggcttatgtccactgggttcgtgccttcatccgtttccacggtgtgcgtcacccggcaaccttgggcagcagcgaagtcga
ggcatttctgtcctggctggcgaacgagcgcaaggtttcggtctccacgcatcgtcaggcattggcggccttgctgttcttctacggcaaggtgct
gtgcacggatctgccctggcttcaggagatcggaagacctcggccgtcgcggcgcttgccggtggtgctgaccccggatgaagtggttcgcat
cctcggttttctggaaggcgagcatcgtttgttcgcccagcttctgtatggaacgggcatgcggatcagtgagggtttgcaactgcgggtcaagg
atctggatttcgatcacggcacgatcatcgtgcgggagggcaagggctccaaggatcgggccttgatgttacccgagagcttggcacccagcc
tgcgcgagcaggggaattaattcccacgggttttgctgcccgcaaacgggctgttctggtgttgctagtttgttatcagaatcgcagatccggcttc
agccggtttgccggctgaaagcgctatttcttccagaattgccatgattttttccccacgggaggcgtcactggctcccgtgttgtcggcagctttga
ttcgataagcagcatcgcctgtttcaggctgtctatgtgtgactgttgagctgtaacaagttgtctcaggtgttcaatttcatgttctagttgctttgtttt
actggtttcacctgttctattaggtgttacatgctgttcatctgttacattgtcgatctgttcatggtgaacagctttgaatgcaccaaaaactcgtaaaag
ctctgatgtatctatcttttttacaccgttttcatctgtgcatatggacagttttccctttgatatgtaacggtgaacagttgttctacttttgtttgttag
tcttgatgcttcactgatagatacaagagccataagaacctcagatccttccgtatttagccagtatgttctctagtgtggttcgttgtttttgcgtgagcc
atgagaacgaaccattgagatcatacttactttgcatgtcactcaaaaattttgcctcaaaactggtgagctgaatttttgcagttaaagcatcgtgtagt
gtttttcttagtccgttatgtaggtaggaatctgatgtaatggttgttggtattttgtcaccattcatttttatctggttgttctcaagttcggttacgaga
tccatttgtctatctagttcaacttggaaaatcaacgtatcagtcgggcggcctcgcttatcaaccaccaatttcatattgctgtaagtgtttaaatcttta
cttattggtttcaaaacccattggttaagccttttaaactcatggtagttattttcaagcattaacatgaacttaaattcatcaaggctaatctctatattt
gccttgtgagttttcttttgtgttagttcttttaataaccactcataaatcctcatagagtatttgttttcaaaagacttaacatgttccagattatatttt
atgaatttttttaactggaaaagataaggcaatatctcttcactaaaaactaattctaatttttcgcttgagaacttggcatagtttgtccactggaaaatc
tcaaagcctttaaccaaaggattcctgatttccacagttctcgtcatcagctctctggttgctttagctaatacaccataagcattttccctactgatgttc
atcatctgagcgtattggttataagtgaacgataccgtccgttctttccttgtagggttttcaatcgtggggttgagtagtgccacacagcataaaattagc
ttggtttcatgctccgttaagtcatagcgactaatcgctagttcatttgctttgaaaacaactaattcagacatacatctcaattggtctaggtgattttaa
tcactataccaattgagatgggctagtcaatgataattactagtccttttcctttgagttgtgggtatctgtaaattctgctagacctttgctggaaaactt
gtaaattctgctagaccctctgtaaattccgctagacctttgtgtgttttttttgtttatattcaagtggttataatttatagaataaagaaagaataaaaa
aagataaaaagaatagatcccagccctgtgtataactcactactttagtcagttccgcagtattacaaaaggatgtcgcaaacgctgtttgctcctctacaa
aacagaccttaaaaccctaaaggcttaagtagcaccctcgcaagctcgggcaaatcgctgaatattccttttgtctccgaccatcaggcacctgagt
cgctgtctttttcgtgacattcagttcgctgcgctcacggctctggcagtgaatgggggtaaatggcactacaggcgccttttatggattcatgcaa
ggaaactacccataatacaagaaaagcccgtcacgggcttctcagggcgttttatggcgggtctgctatgtggtgctatctgactttttgctgttcag
cagttcctgccctctgattttccagtctgaccacttcggattatcccgtgacaggtcattcagactggctaatgcacccagtaaggcagcggtatcat
caacaggcttacccgtcttactgtcgggaattcgcgttggccgattcattaatgcagattctgaaatgagctgttgacaattaatcatccggctcgtat
aatgtgtggaattgtgagcggataacaatttcacacaggaaacagcgccgctgagaaaaagcgaagcggcactgctctttaacaatttatcagac
aatctgtgtgggcactcgaccggaattatcgattaactttattattaaaaattaaagaggtatatattaatgtatcgattaaataaggaggaataaacc
atgaccgaaactcatgcaattgctggtgtaccgatgcgttgggttggcccgctgcgtatctctggcaacgtagcagaaaccgaaactcaggttcc
gctggcaacctacgaatctccgctgtggccgtctgtgggtcgtggtgcgaaggtatcccgtctgaccgaaaagggtatcgttgcaaccctcgttg
acgaacgtatgacccgtagcgtcatcgtagaagccaccgacgcccagaccgcttatatggctgcacagaccattcatgctcgcattgatgaact
gcgcgaagtggttcgtggttgttcccgtttcgcgcagctgattaatatcaaacatgaaattaacgccaacttgctgttcatccgctttgaatttaccac
cggtgacgcatctggccataacatggccaccctggcgtctgacgttctgttgggtcacctcctggaaaccatcccaggcatctcctacggctcca
tttctggcaactactgcactgacaagaaagcaactgccattaacggtatcctgggtcgcggcaaaaacgttatcaccgaattgctggtgccgcgc
gatgttgtagaaaataacctgcacaccaccgctgcgaaaatcgttgaactgaacatccgtaaaaacctgctgggcactctcctggcaggcggca
tccgttctgcgaacgcgcattttgcaaatatgctgctgggtttctacctggcaactggccaggacgcagcaaacatcgttgagggtagccagggc
gttgtaatggcggaagatcgtgacggtgatctgtacttcgcgtgcacccttccgaatctgatcgtgggtactgttggcaacggtaagggcctgggt
tttgttgaaacgaatctggcccgtctgggctgtcgcgcggatcgcgaaccgggcgaaaatgcgcgccgtctggccgttatcgccgctgccacc
gtgctgtgtggtgaactgtccctgttggccgcgcagaccaaccctggtgaactgatgcgtgcacatgttcagctggaacgtgataacaaaaccg
cgaaagttggtgcgtaatctagacgcactaggaggatataccaatgagcatttctatcggtatccatgatctttcgttcgcgactaccgagttcgttc
tgcctcatacggctctggccgagtataatggtactgaaattggtaaatatcacgttggcatcggtcaacagagcatgagcgtacctgcggcggat
gaggacatcgtgactatggccgcgaccgccgcacgtccgatcattgaacgcaacggtaaatcccgtatccgcactgtggtgttcgctacggagt
cctccattgatcaagctaaagcgggtggcgtatacgtacattctctgctgggcctggaatccgcgtgccgtgtagttgaactgaaacaggcatgct
acggtgcgacggcagcattgcagttcgctattggtctggttcgccgcgatccggcgcagcaggtgctggtgatcgcaagcgacgtttctaaata
cgagttggattcgccgggtgaggcaacccagggcgcagcggccgttgctatgctggttggcgcagatcctgcactgcttcgcattgaagaacc
gtccggcctcttcaccgcggacgttatggatttctggcgcccgaactacctgaccaccgccctggtagatggccaggagtccatcaacgcctac
ctgcaagcagttgaaggtgcgtggaaggactacgcagagcaggacggtcgtagccttgaagagtttgcagctttcgtttaccaccagccgttca
ctaaaatggcgtacaaagctcaccgccacctgctgaacttcaacggttacgacactgacaaagatgcgattgaaggcgccctgggtcaaactac
cgcttacaataatgtgatcggtaactcctacactgcgtcagtttatctgggcctggcggcgcttctggatcaggcggacgacctgaccggtcgttc
tatcggcttcctgtcttacggctccggtagcgtagcggagtttttctcgggcaccgttgttgctggttaccgtgaacgtctgcgtactgaagcgaac
caggaagctatcgcacgccgtaaatctgtcgattacgcgacttatcgtgaactgcatgagtacacgctcccgtctgatggtggtgatcacgccac
gccggttcagaccaccggtccattccgtctggcaggtatcaacgaccataaacgtatctatgaagcacgttaaagatctcgcactaggaggatat
accaatgaccgacgtgcgctttcggataattggtaccggtgcgtacgtgccggaacgcatcgtgagtaatgacgaagttggcgcaccggctggt
gtggacgatgattggattacacgcaagacgggtatacgtcagagacggtgggcggcagatgatcaagcgactagtgaccttgccactgccgc
gggtcgtgcggcactgaaggctgcgggcataactccggagcagttaaccgttattgcggtggctacttcgaccccggaccgtcctcagccccct
accgctgcttatgttcagcatcatttgggtgcaacaggcaccgccgcattcgatgtgaacgcagtgtgcagcgggacggttttcgcactttcgtca
gttgcgggtacgttggtttaccgcggcggatacgctctggtaataggggcggacctgtattcgcgtatcctgaaccctgcggatcgtaaaacagt
ggtactttttggtgacggggctggtgcgatggtattaggcccgacgtccactggaaccgggccaatcgtgcgccgggtcgctcttcataccttcg
gaggactgacggatctgatccgcgtgccagcaggtggttcacggcaacctcttgacacagacgggcttgatgccggtcttcaatactttgcaatg
gatggtcgcgaagtgcggcgtttcgtgacagagcaccttccgcagctgattaaggggtttctgcatgaagcaggagtggatgcggcagacattt
cgcactttgtcccacaccaggcgaacggcgttatgctggatgaagtgttcggcgaattgcatctccctcgtgccactatgcaccgtacagttgaaa
catacggtaatacaggcgctgcgtccatacctatcactatggatgcagcagtgcgcgctgggtcctttcgcccaggcgaactcgttctgttggca
ggtttcggcgggggcatggcagcttccttcgcgttgattgaatggtga

### Example 6

### Isoprenoid Production Via Acetoacetyl-CoA Synthase (NphT7) Using Engineered Strains

(i) Materials

### TM3 Media Recipe (Per Liter Fermentation Media):

K₂HPO₄ 13.6 g, KH₂PO₄ 13.6 g, MgSO₄*7H₂O 2 g, citric acid monohydrate 2 g, ferric ammonium citrate 0.3 g, (NH₄)₂SO₄ 3.2 g, yeast extract 0.2 g, 1000× Trace Metals Solution 1 ml. All of the components are added together and dissolved in diH₂O. The pH is adjusted to 6.8 with ammonium hydroxide (30%) and brought to volume. Media is then filter-sterilized with a 0.22 micron filter. Glucose 10.0 g and antibiotics are added after sterilization and pH adjustment.

### 1000× Trace Metal Solution (Per Liter Fermentation Media):

Citric Acid*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, NaMoO₄*2H₂O 100 mg. Each component is dissolved one at a time in diH₂O. The pH is adjusted to 3.0 with HCl/NaOH, and then the solution is brought to volume and filter-sterilized with a 0.22 micron filter.

(ii) Experimental Procedure

Cells are grown overnight in Luria-Bertani broth+antibiotics. The day after, they are diluted to an OD600 of 0.05 in 20 mL TM3 medium containing 50 ug/ml of spectinomycin and 50 ug/mL carbenicillin (in a 250-mL baffled Erlenmeyer flask), and incubated at 34° C. and 200 rpm. Prior to inoculation, an overlay of 20% (v/v) dodecane (Sigma-Aldrich) is added to the culture flask to trap the volatile sesquiterpene product as described previously (Newman et. al., Biotechnol. Bioeng. 95:684-691, 2006).

After 2 h of growth, OD600 is measured and 0.05-0.40 mM isopropyl β-d-1-thiogalactopyranoside (IPTG) is added. Samples are taken regularly during the course of the fermentation. At each time point, OD600 is measured. Also, isoprenoid concentration in the organic layer is assayed by diluting the dodecane overlay into ethyl acetate. Dodecane/ethyl acetate extracts are analyzed by GC-MS methods as previously described (Martin et. al., Nat. Biotechnol. 2003, 21:96-802). Isoprenoid samples of known concentration are injected to produce standard curves for isoprenoid. The amount of isoprenoid per sample is calculated using the isoprenoid standard curves.

### Example 7

### Production of Isoprene by Saccharomyces cerevisiae Engineered to have Acetoacetyl-CoA Synthase (NphT7) Activity

Yeast strains are generated by transformation of the abovementioned plasmids into a parent strain using the protocol described in the s.c. EasyComp Transformation kit (Invitrogen). Yeast strains harboring the plasmid are selected for and maintained on SC Minimal Medium with 2% glucose supplemented with the indicated selective marker. Isolated colonies harboring the plasmid are chosen for further experimentation.

The specific productivity of isoprene from the engineered yeast strains is determined. To induce expression of the genes encoded by the plasmid, cultures are grown overnight in liquid SC Minimal Medium supplemented with the selective marker. The cultures are then diluted to an OD₆₀₀ of approximately 0.2 and grown for 2-3 hours. A 100 μL sample of the broth is incubated in a 2 mL headspace vial at 34° C. for 30 minutes, followed by heat kill at 70° C. for 12 minutes. Levels of isoprene in the headspace are determined, for example, by flame ionization detector coupled to a gas chromatograph (Model G1562A, Agilent Technologies) (Mergen et al., LC GC North America, 28(7):540-543, 2010).

### Example 8

### Improving Isoprene Production with Acetoacetyl-CoA Synthase (nphT7) Utilizing the Upper MVA Pathway Enzymes Derived from Streptococcus suis in E. coli

Generation of plasmid pMCM1221

Plasmid encoding an MVA upper pathway was constructed by GeneOracle (Mountain View, Calif.) using the following design. A synthetic DNA encoding Acetyl-CoA-acetyltransferase-RBS-3-hydroxy-3-methylglutaryl-CoA-synthase-RBS-hydroxymethylglutaryl-CoA-reductase was created and then cloned into pMCM82 (see U.S. Patent Appl. Pub. No. US 2011/0159557) between the NcoI and PstI sites, replacing the existing operon. The vector provided an RBS for mvaE. See FIG. 5 for plasmid map.

TABLE 8
Description of pMCM1221

| Plasmid | Source | Acetyl- |
|---|---|---|
| Name | Organsim | CoA_acetyltransferase |
| pCL-Ptrc- | Streptococcus | gi\|146321498\|ref\|YP_001201209.1\|_Acetyl- |
| Upper_GcMM_159 | suis | CoA_acetyltransferase_[Streptococcus_suis_98HAH33] |
| (Streptococcus | | |
| suis) | | |

| Plasmid | 3-hydroxy-3- |
|---|---|
| Name | methylglutaryl_CoA_synthase |
| pCL-Ptrc- | gi\|146321499\|ref\|YP_001201210.1\|_3- |
| Upper_GcMM_159 | hydroxy-3- |
| (Streptococcus | methylglutaryl_CoA_synthase_[Streptococcus_suis_98HAH33] |
| suis) | |

| Plasmid | hydroxymethylglutaryl- | Origin and |
|---|---|---|
| Name | CoA_reductase | Selection |
| pCL-Ptrc- | gi\|146321500\|ref\|YP_001201211.1\|_hydroxymethylglutaryl- | pSC101, |
| Upper_GcMM_159 | CoA_reductase_[Streptococcus_suis_98HAH33] | Spectinomycin |
| (Streptococcus | | (50 ug/mL) |
| suis) | | |

DNA sequence of plasmid construct pMCM1221-pCL-Ptrc-Upper_GcMM_{—}159 (Streptococcus suis).

(SEQ ID NO: 25)
cccgtcttactgtcgggaattcgcgttggccgattcattaatgcagattctgaaatgagctgttgacaattaatcatccggctcgtataatgtgtggaa
ttgtgagcggataacaatttcacacaggaaacagcgccgctgagaaaaagcgaagcggcactgctctttacaatttatcagacaatctgtgtggg
cactcgaccggaattatcgattaactttattattaaaaattaaagaggtatatattaatgtatcgattaaataaggaggaataaaccatgagcacgttta
gtggtttttacaagaaaagtcgccaggagcgcatcgatatcttacgtcagaaccggtccctggcagaagattctctggatattctgtacaaggacg
aaaacctgcctgaagctatcgcgggcaaaatggccgaaaaccacttggggacgttcagcctgcccttctcggtactgcctgagctgctggtaga
tgggcagacatactctgttcctatggtaactgaggagcctagcgtggtagcagccgcctcgttcggggcaaaaattatcgcgaaatccggtggct
ttacaacaaccatccacaaccgtataatgatcggccaggtagcgttatatgatataagtgaccattctcgcgccacgcaagcaattctggatcaca
aggagagtatacttgaaaacgctaaccaagctcatcccagtatagtcaaacgcggcggaggggctagagagcttacagttgagtctaaggatg
aatttctgatcgtctaccttcaggtagatgtgcaagaagcaatgggtgcaaacatactgaacaacatgcttgaagccgtgaaagatgatctggaag
aactttccaaaggccaggcgcttatgggaatcctcagcaactacgccaccgagtcattaatcacagcacagtgtcatatcgcaatatcaagcctg
gcgacttcctctgccattgctcaggagaccgcccagaaaattgcactcgcgagcaaattagcgcaagttgacccatatcgtgccgcgacacaca
ataaaggtatttttaatgggattgacgctgtcgtcattgcagctgggaacgactggcgtgctgttgaggcaggtgctcatgcgtatgccagccgcg
atggacaatataaaggcctgagtacctggtcgatcgatggggaacacttagttggctccattacattgccgttgcctatagcttcagttggcggaa
gtataggcctgaatccgaaggttgccgtcgcatttgacttactgcaacagccaaaagcacgccaattagccagcattattgcctcagtgggtctct
gccaaaatttcgctgctctccgggcgctggtaactagtggtattcaggccggtcacatgaagttacatgctaaatcgctggctttactggccgggg
cggaagaacatgaggtcgaccaactggctcagctgctccgcaaggaaaaacattccaatcttgaaacggctcagaacttactggcaaaaatgc
gtgaacattgggaatgggaaagacatccttgatctagacgcactaggaggatataccaatgcgtaaacaagttaacctttcttttgtgcataaggg
ctacattatgaacattggtattgacaagatcggtttcgcagcccccgattatgtgttagacttagcggatttagcccaggcgcgtaatgttgatccta
acaaatttaagatcggtttacttcagagcgagatggccgtcgccccggtgacgcaggatatcatatcgttaggcgccaaagctgccgaggcaatt
ctgaccgaagaggacaaacaaacgatcgatatggttatcgtgggcaccgaaagttccgtcgatcaaagcaaagccgcggcggtgacaataca
cggactgttaggcatccagcctttcgctcggtcgattgaaatgaaggaggcttgttatggcgcgactgccggattaagtttggctaagtctcatatc
gcccagtttcctgaaagcaaagttttagtcatagcctcggatattgctaaatatggggtagcttccggaggtgaacctactcagggtgcgggagcc
gtggcaatgcttgtgacggcgaaccctcggattctggtgttgaacaacgataatgtctgccaaacgcgcgatatatacgatttttggcgtccgaatt
atgacaagtacccacgtgtcgacggcaagttctccaccgaacagtatacagactgcttaactactacattcgattactatcaacagaagacgggg
aaaaccctgaacgactttgccgcaatgtgcttgcacatccccttctctaaacaaggtctgaaaggcttacaggcgattgcccaagatgaagaaac
cctcagccggttaacggagcgcttccaggaagccattgtctacaacaaagtggtggggaatatctataccggcagcattttcttgagtctcctgtct
ctcctggaaaattcacgggctctggaaacaggagatcagattctgttctacagttacggctctggtgctgtatgcgaaattttctctggacaactggt
cgaagggtaccgcaatcacctgcaagagaatcgcctggaacagttgaaccagcgtaccaaactgtccgtcaaggaatacgaacaggtgtttttt
gaggaaataaccctggatgaaactgggtcctccttggatctcccagaagatcagtccccgttcgcgcttattaaagtcgataaccacaaacgtatc
tatcgtaaatgaagatctcgcactaggaggatataccaatgaagaaagtcgcgatcgtctctgcttaccggagcgcaatcgggtcctttggcgga
tcattgaaggacatcgagattgccgatttaggcgcacaggtattggagacggccctcgcctcgaagaacataccggcggatagcgtggacgaa
gttatcttcggtaacgtactgtccgccggtcaaggtcagaacatagcaagacagatagccatacgtgccgggattccacaaacagcatcagcgt
acgccgtaaataaggtgtgtggttcgggtttaaaatcagtgcttctggcggcacagtcgattatgctgggcgacaacgatgtcgtcgttgcaggtg
gaattgaaatcatgtcccaggcaccgtatttgtcaaaaactagtcgctttgggagtaagttcgggcacatcacccttgaagattccatgctgacgga
tggtctcactgatgcgtttaatgactaccacatgggcatcactgcggaaaatgtagcggagcattatcaggtaagccgtgccgagcaggacgcct
tcacttactcatctcaggaaaaagctgccaaggcgatagcagaaggacgttttgtggacgaaatcgttcctatccgtctcaaaaaccgcaagggc
gaaacgatattcgcaactgatgagtatcctagactgaccccaattgaaaagttagctaccctccgcccatcttttaagaaggatggaaccgtcaca
gcagcaaacgcctccggcattaatgacgggtgcgcggtgctgatacttatgtcagacgagaaagcgtccgagttaaacatccagccgctgacat
acatagaagcctatgcaacctcgggtctcgatcccgcccttatgggactgggtccgattaccgcctctcaaaaagcccttcagaaattgaacaaa
acggttgaagacatcgacctgtttgaattaaatgaagcgttcgctgcacagagtattcccgttgtgaaacagctcgggatcgatccggcgaaagt
caacgttaacggtggggcaatagctttaggtcaccccatcggcgcttcaggtagccgcattcttgtcacgcttatccacgaactcatcaaacaaga
gaaagaactcggcttatgctcactgtgtattggcggcggtcaggggattagtctgatagtatccaacgctcaaacttcctgactgcagctggtacc
atatgggaattcgaagcttgggcccgaacaaaaactcatctcagaagaggatctgaatagcgccgtcgaccatcatcatcatcatcattgagttta
aacggtctccagcttggctgttttggcggatgagagaagattttcagcctgatacagattaaatcagaacgcagaagcggtctgataaaacagaat
ttgcctggcggcagtagcgcggtggtcccacctgaccccatgccgaactcagaagtgaaacgccgtagcgccgatggtagtgtggggtctccc
catgcgagagtagggaactgccaggcatcaaataaaacgaaaggctcagtcgaaagactgggcctttcgttttatctgttgtttgtcggtgaacgc
tctcctgagtaggacaaatccgccgggagcggatttgaacgttgcgaagcaacggcccggagggtggcgggcaggacgcccgccataaact
gccaggcatcaaattaagcagaaggccatcctgacggatggcctttttgcgtttctacaaactctttttgtttatttttctaaatacattcaaatatgtatc
cgctcatgagacaataaccctgataaatgcttcaataatctggcgtaatagcgaagaggcccgcaccgatcgcccttcccaacagttgcgcagc
ctgaatggcgaatggcgcctgatgcggtattttctccttacgcatctgtgcggtatttcacaccgcatatggtgcactctcagtacaatctgctctgat
gccgcatagttaagccagccccgacacccgccaacacccgctgacgagcttagtaaagccctcgctagattttaatgcggatgttgcgattactt
cgccaactattgcgataacaagaaaaagccagcctttcatgatatatctcccaatttgtgtagggcttattatgcacgcttaaaaataataaaagcag
acttgacctgatagtttggctgtgagcaattatgtgcttagtgcatctaacgcttgagttaagccgcgccgcgaagcggcgtcggcttgaacgaatt
gttagacattatttgccgactaccttggtgatctcgcctttcacgtagtggacaaattcttccaactgatctgcgcgcgaggccaagcgatcttcttct
tgtccaagataagcctgtctagcttcaagtatgacgggctgatactgggccggcaggcgctccattgcccagtcggcagcgacatccttcggcg
cgattttgccggttactgcgctgtaccaaatgcgggacaacgtaagcactacatttcgctcatcgccagcccagtcgggcggcgagttccatagc
gttaaggtttcatttagcgcctcaaatagatcctgttcaggaaccggatcaaagagttcctccgccgctggacctaccaaggcaacgctatgttctc
ttgcttttgtcagcaagatagccagatcaatgtcgatcgtggctggctcgaagatacctgcaagaatgtcattgcgctgccattctccaaattgcagt
tcgcgcttagctggataacgccacggaatgatgtcgtcgtgcacaacaatggtgacttctacagcgcggagaatctcgctctctccaggggaag
ccgaagtttccaaaaggtcgttgatcaaagctcgccgcgttgtttcatcaagccttacggtcaccgtaaccagcaaatcaatatcactgtgtggctt
caggccgccatccactgcggagccgtacaaatgtacggccagcaacgtcggttcgagatggcgctcgatgacgccaactacctctgatagttg
agtcgatacttcggcgatcaccgcttccctcatgatgtttaactttgttttagggcgactgccctgctgcgtaacatcgttgctgctccataacatcaa
acatcgacccacggcgtaacgcgcttgctgcttggatgcccgaggcatagactgtaccccaaaaaaacagtcataacaagccatgaaaaccgc
cactgcgccgttaccaccgctgcgttcggtcaaggttctggaccagttgcgtgagcgcatacgctacttgcattacagcttacgaaccgaacagg
cttatgtccactgggttcgtgccttcatccgtttccacggtgtgcgtcacccggcaaccttgggcagcagcgaagtcgaggcatttctgtcctggct
ggcgaacgagcgcaaggtttcggtctccacgcatcgtcaggcattggcggccttgctgttcttctacggcaaggtgctgtgcacggatctgccct
ggcttcaggagatcggaagacctcggccgtcgcggcgcttgccggtggtgctgaccccggatgaagtggttcgcatcctcggttttctggaag
gcgagcatcgtttgttcgcccagcttctgtatggaacgggcatgcggatcagtgagggtttgcaactgcgggtcaaggatctggatttcgatcac
ggcacgatcatcgtgcgggagggcaagggctccaaggatcgggccttgatgttacccgagagcttggcacccagcctgcgcgagcagggga
attaattcccacgggttttgctgcccgcaaacgggctgttctggtgttgctagtttgttatcagaatcgcagatccggcttcagccggtttgccggct
gaaagcgctatttcttccagaattgccatgattttttccccacgggaggcgtcactggctcccgtgttgtcggcagctttgattcgataagcagcatc
gcctgtttcaggctgtctatgtgtgactgttgagctgtaacaagttgtctcaggtgttcaatttcatgttctagttgctttgttttactggtttcacctgttct
attaggtgttacatgctgttcatctgttacattgtcgatctgttcatggtgaacagctttgaatgcaccaaaaactcgtaaaagctctgatgtatctatctt
ttttacaccgttttcatctgtgcatatggacagttttccctttgatatgtaacggtgaacagttgttctacttttgtttgttagtcttgatgcttcactgataga
tacaagagccataagaacctcagatccttccgtatttagccagtatgttctctagtgtggttcgttgtttttgcgtgagccatgagaacgaaccattga
gatcatacttactttgcatgtcactcaaaaattttgcctcaaaactggtgagctgaatttttgcagttaaagcatcgtgtagtgtttttcttagtccgttatg
taggtaggaatctgatgtaatggttgttggtattttgtcaccattcatttttatctggttgttctcaagttcggttacgagatccatttgtctatctagttcaa
cttggaaaatcaacgtatcagtcgggcggcctcgcttatcaaccaccaatttcatattgctgtaagtgtttaaatctttacttattggtttcaaaacccat
tggttaagccttttaaactcatggtagttattttcaagcattaacatgaacttaaattcatcaaggctaatctctatatttgccttgtgagttttcttttgtgtt
agttcttttaataaccactcataaatcctcatagagtatttgttttcaaaagacttaacatgttccagattatattttatgaatttttttaactggaaaagata
aggcaatatctcttcactaaaaactaattctaatttttcgcttgagaacttggcatagtttgtccactggaaaatctcaaagcctttaaccaaaggattc
ctgatttccacagttctcgtcatcagctctctggttgctttagctaatacaccataagcattttccctactgatgttcatcatctgagcgtattggttataa
gtgaacgataccgtccgttctttccttgtagggttttcaatcgtggggttgagtagtgccacacagcataaaattagcttggtttcatgctccgttaagt
catagcgactaatcgctagttcatttgctttgaaaacaactaattcagacatacatctcaattggtctaggtgattttaatcactataccaattgagatg
ggctagtcaatgataattactagtccttttcctttgagttgtgggtatctgtaaattctgctagacctttgctggaaaacttgtaaattctgctagaccctc
tgtaaattccgctagacctttgtgtgttttttttgtttatattcaagtggttataatttatagaataaagaaagaataaaaaaagataaaaagaatagatcc
cagccctgtgtataactcactactttagtcagttccgcagtattacaaaaggatgtcgcaaacgctgtttgctcctctacaaaacagaccttaaaacc
ctaaaggcttaagtagcaccctcgcaagctcgggcaaatcgctgaatattccttttgtctccgaccatcaggcacctgagtcgctgtctttttcgtga
cattcagttcgctgcgctcacggctctggcagtgaatgggggtaaatggcactacaggcgccttttatggattcatgcaaggaaactacccataat
acaagaaaagcccgtcacgggcttctcagggcgttttatggcgggtctgctatgtggtgctatctgactttttgctgttcagcagttcctgccctctg
attttccagtctgaccacttcggattatcccgtgacaggtcattcagactggctaatgcacccagtaaggcagcggtatcatcaacaggctta

### Example 9

### Preparation of an Acetoacetyl-CoA Synthase (NphT7) Containing Plasmid

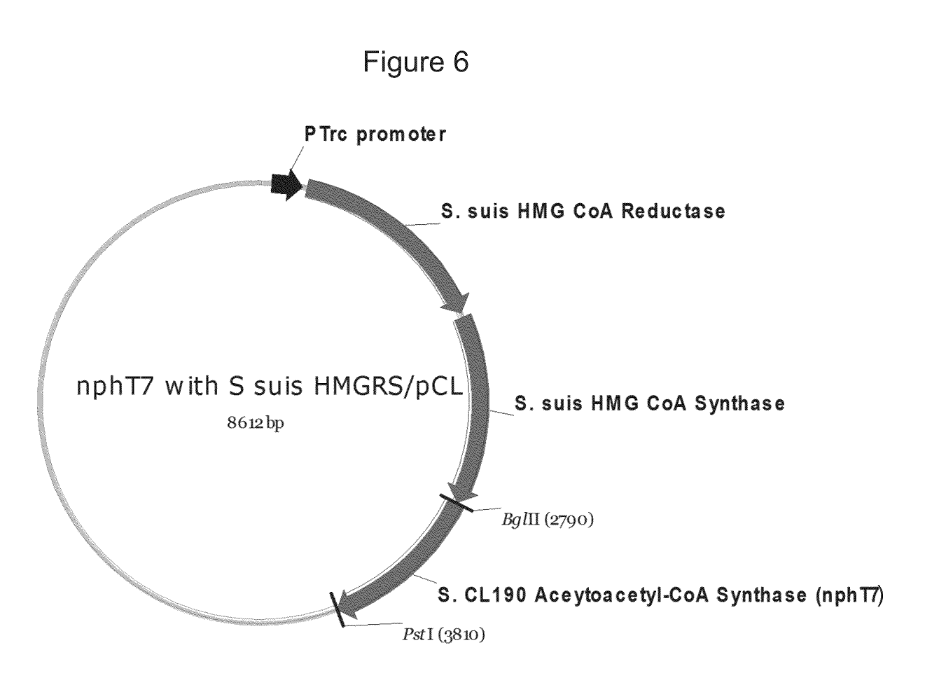

A three component upper MVA pathway derived from Streptococcus suis and harbored by plasmid construct pMCM1221 (pCL-Ptrc-Upper_GcMM_{—}159) was used. Acetoacetyl-CoA Synthase derived from Streptomyces sp. strain CL190 and encoded by nphT7 within plasmid construct MCM1187 has been described previously (see Table 2). The nphT7gene was PCR amplified using PfuUltra II Fusion HS DNA Polymerase (Agilent Technologies, Santa Clara, Calif.) from the MCM1187 template using primers 5′ BglII rbs nphT7 primer and 3′ PstI nphT7 primer according to the manufacturer's suggested protocol. Using standard molecular biology techniques: the nphT7-containing PCR product was verified via agarose gel electrophoresis (E-gel 0.8% (GP), Invitrogen); the PCR reaction was then cleaned using QIAquick PCR purification Kit (Qiagen, Germantown, Md.); both the clean PCR product and an aliquot of purified pMCM1221 were cut using Bgl II and Pst I (Roche, Indianapolis, Ind.); the completed restriction digests were cleaned using QIAquick Gel Extraction Kit (Qiagen, Germantown, Md.); and the resulting clean Bgl II-Pst I fragments were ligated using T4 DNA ligase from New England Biolabs. The ligation was later transformed into electroporation competent Top10 cells (Invitrogen, Carlsbad, Calif.) using a Bio-Rad a 0.1 cm electrode gap cuvette and the Bio-Rad Gene Pulser system (Bio-Rad Laboratories, Hercules, Calif.). Transformed cells were selected on LB media containing 50 ug/ml spectinomycin (Teknova, Hollister, Calif.). Plasmid was prepared from cultures generated by spectinomycin resistant colonies using a QIAprep Spin Miniprep Kit (Qiagen, Germantown, Md.) along with the suggested protocol. The subsequent positive Top10 clone harboring the nphT7-containing plasmid construct, now designated strain REM B5_{—}25, was identified via DNA sequence analysis (Sequetech, Mountain View, Calif.) utilizing primers nphT7 top seq primer, nphT7 bot seq primer, pSE3803 (Sequetech in-house primer), 5′ BglII rbs nphT7 primer, and 3′ PstI nphT7 primer. The nphT7-containing plasmid construct has been named “nphT7 with S suis HMGRS/pCL” and is illustrated in FIG. 6.

Primer sequences used to create and verify plasmid construct nphT7 with S suis HMGRS/pCL.

5′ BglII rbs nphT7 primer:
(SEQ ID NO: 26)
5′- GGGCagatctcgcactaggaggatataccaatgaccgacgtgcgct
ttcgg
3′ PstI nphT7 primer:
(SEQ ID NO: 27)
5′- TATCCTGCAG tcaccattcaatcaacgcgaaggaagc
nphT7 top seq primer:
(SEQ ID NO: 28)
5′- CGGCACTGAAGGCTGCGG
nphT7 bottom seq primer:
(SEQ ID NO: 29)
5′- CCGCAGCCTTCAGTGCCG
(Sequetech in house primer) pSE3803:
(SEQ ID NO: 30)
5′- GGCATGGGGTCAGGTGGG

DNA sequence of plasmid construct nphT7 with S suis HMGRS/pCL.

(SEQ ID NO: 31)
5′- cccgtcttactgtcgggaattcgcgttggccgattcattaatgcagattctgaaatgagctgttgacaattaatcatccggctcgtataatgtg
tggaattgtgagcggataacaatttcacacaggaaacagcgccgctgagaaaaagcgaagcggcactgctctttacaatttatcagacaatctgt
gtgggcactcgaccggaattatcgattaactttattattaaaaattaaagaggtatatattaatgtatcgattaaataaggaggaataaaccatgagca
cgtttagtggtttttacaagaaaagtcgccaggagcgcatcgatatcttacgtcagaaccggtccctggcagaagattctctggatattctgtacaa
ggacgaaaacctgcctgaagctatcgcgggcaaaatggccgaaaaccacttggggacgttcagcctgcccttctcggtactgcctgagctgct
ggtagatgggcagacatactctgttcctatggtaactgaggagcctagcgtggtagcagccgcctcgttcggggcaaaaattatcgcgaaatcc
ggtggctttacaacaaccatccacaaccgtataatgatcggccaggtagcgttatatgatataagtgaccattctcgcgccacgcaagcaattctg
gatcacaaggagagtatacttgaaaacgctaaccaagctcatcccagtatagtcaaacgcggcggaggggctagagagcttacagttgagtcta
aggatgaatttctgatcgtctaccttcaggtagatgtgcaagaagcaatgggtgcaaacatactgaacaacatgcttgaagccgtgaaagatgatc
tggaagaactttccaaaggccaggcgcttatgggaatcctcagcaactacgccaccgagtcattaatcacagcacagtgtcatatcgcaatatca
agcctggcgacttcctctgccattgctcaggagaccgcccagaaaattgcactcgcgagcaaattagcgcaagttgacccatatcgtgccgcga
cacacaataaaggtatttttaatgggattgacgctgtcgtcattgcagctgggaacgactggcgtgctgttgaggcaggtgctcatgcgtatgcca
gccgcgatggacaatataaaggcctgagtacctggtcgatcgatggggaacacttagttggctccattacattgccgttgcctatagcttcagttg
gcggaagtataggcctgaatccgaaggttgccgtcgcatttgacttactgcaacagccaaaagcacgccaattagccagcattattgcctcagtg
ggtctctgccaaaatttcgctgctctccgggcgctggtaactagtggtattcaggccggtcacatgaagttacatgctaaatcgctggctttactgg
ccggggcggaagaacatgaggtcgaccaactggctcagctgctccgcaaggaaaaacattccaatcttgaaacggctcagaacttactggcaa
aaatgcgtgaacattgggaatgggaaagacatccttgatctagacgcactaggaggatataccaatgcgtaaacaagttaacctttcttttgtgcat
aagggctacattatgaacattggtattgacaagatcggtttcgcagcccccgattatgtgttagacttagcggatttagcccaggcgcgtaatgttg
atcctaacaaatttaagatcggtttacttcagagcgagatggccgtcgccccggtgacgcaggatatcatatcgttaggcgccaaagctgccgag
gcaattctgaccgaagaggacaaacaaacgatcgatatggttatcgtgggcaccgaaagttccgtcgatcaaagcaaagccgcggcggtgac
aatacacggactgttaggcatccagcctttcgctcggtcgattgaaatgaaggaggcttgttatggcgcgactgccggattaagtttggctaagtct
catatcgcccagtttcctgaaagcaaagttttagtcatagcctcggatattgctaaatatggggtagcttccggaggtgaacctactcagggtgcgg
gagccgtggcaatgcttgtgacggcgaaccctcggattctggtgttgaacaacgataatgtctgccaaacgcgcgatatatacgatttttggcgtc
cgaattatgacaagtacccacgtgtcgacggcaagttctccaccgaacagtatacagactgcttaactactacattcgattactatcaacagaaga
cggggaaaaccctgaacgactttgccgcaatgtgcttgcacatccccttctctaaacaaggtctgaaaggcttacaggcgattgcccaagatgaa
gaaaccctcagccggttaacggagcgcttccaggaagccattgtctacaacaaagtggtggggaatatctataccggcagcattttcttgagtctc
ctgtctctcctggaaaattcacgggctctggaaacaggagatcagattctgttctacagttacggctctggtgctgtatgcgaaattttctctggaca
actggtcgaagggtaccgcaatcacctgcaagagaatcgcctggaacagttgaaccagcgtaccaaactgtccgtcaaggaatacgaacaggt
gttttttgaggaaataaccctggatgaaactgggtcctccttggatctcccagaagatcagtccccgttcgcgcttattaaagtcgataaccacaaa
cgtatctatcgtaaatgaagatctcgcactaggaggatataccaatgaccgacgtgcgctttcggataattggtaccggtgcgtacgtgccggaa
cgcatcgtgagtaatgacgaagttggcgcaccggctggtgtggacgatgattggattacacgcaagacgggtatacgtcagagacggtgggc
ggcagatgatcaagcgactagtgaccttgccactgccgcgggtcgtgcggcactgaaggctgcgggcataactccggagcagttaaccgttat
tgcggtggctacttcgaccccggaccgtcctcagccccctaccgctgcttatgttcagcatcatttgggtgcaacaggcaccgccgcattcgatgt
gaacgcagtgtgcagcgggacggttttcgcactttcgtcagttgcgggtacgttggtttaccgcggcggatacgctctggtaataggggcggac
ctgtattcgcgtatcctgaaccctgcggatcgtaaaacagtggtactttttggtgacggggctggtgcgatggtattaggcccgacgtccactgga
accgggccaatcgtgcgccgggtcgctcttcataccttcggaggactgacggatctgatccgcgtgccagcaggtggttcacggcaacctcttg
acacagacgggcttgatgccggtcttcaatactttgcaatggatggtcgcgaagtgcggcgtttcgtgacagagcaccttccgcagctgattaag
gggtttctgcatgaagcaggagtggatgcggcagacatttcgcactttgtcccacaccaggcgaacggcgttatgctggatgaagtgttcggcg
aattgcatctccctcgtgccactatgcaccgtacagttgaaacatacggtaatacaggcgctgcgtccatacctatcactatggatgcagcagtgc
gcgctgggtcctttcgcccaggcgaactcgttctgttggcaggtttcggcgggggcatggcagcttccttcgcgttgattgaatggtgactgcag
ctggtaccatatgggaattcgaagcttgggcccgaacaaaaactcatctcagaagaggatctgaatagcgccgtcgaccatcatcatcatcatca
ttgagtttaaacggtctccagcttggctgttttggcggatgagagaagattttcagcctgatacagattaaatcagaacgcagaagcggtctgataa
aacagaatttgcctggcggcagtagcgcggtggtcccacctgaccccatgccgaactcagaagtgaaacgccgtagcgccgatggtagtgtg
gggtctccccatgcgagagtagggaactgccaggcatcaaataaaacgaaaggctcagtcgaaagactgggcctttcgttttatctgttgtttgtc
ggtgaacgctctcctgagtaggacaaatccgccgggagcggatttgaacgttgcgaagcaacggcccggagggtggcgggcaggacgccc
gccataaactgccaggcatcaaattaagcagaaggccatcctgacggatggcctttttgcgtttctacaaactctttttgtttatttttctaaatacattc
aaatatgtatccgctcatgagacaataaccctgataaatgcttcaataatctggcgtaatagcgaagaggcccgcaccgatcgcccttcccaaca
gttgcgcagcctgaatggcgaatggcgcctgatgcggtattttctccttacgcatctgtgcggtatttcacaccgcatatggtgcactctcagtaca
atctgctctgatgccgcatagttaagccagccccgacacccgccaacacccgctgacgagcttagtaaagccctcgctagattttaatgcggatg
ttgcgattacttcgccaactattgcgataacaagaaaaagccagcctttcatgatatatctcccaatttgtgtagggcttattatgcacgcttaaaaata
ataaaagcagacttgacctgatagtttggctgtgagcaattatgtgcttagtgcatctaacgcttgagttaagccgcgccgcgaagcggcgtcggc
ttgaacgaattgttagacattatttgccgactaccttggtgatctcgcctttcacgtagtggacaaattcttccaactgatctgcgcgcgaggccaag
cgatcttcttcttgtccaagataagcctgtctagcttcaagtatgacgggctgatactgggccggcaggcgctccattgcccagtcggcagcgac
atccttcggcgcgattttgccggttactgcgctgtaccaaatgcgggacaacgtaagcactacatttcgctcatcgccagcccagtcgggcggcg
agttccatagcgttaaggtttcatttagcgcctcaaatagatcctgttcaggaaccggatcaaagagttcctccgccgctggacctaccaaggcaa
cgctatgttctcttgcttttgtcagcaagatagccagatcaatgtcgatcgtggctggctcgaagatacctgcaagaatgtcattgcgctgccattct
ccaaattgcagttcgcgcttagctggataacgccacggaatgatgtcgtcgtgcacaacaatggtgacttctacagcgcggagaatctcgctctct
ccaggggaagccgaagtttccaaaaggtcgttgatcaaagctcgccgcgttgtttcatcaagccttacggtcaccgtaaccagcaaatcaatatc
actgtgtggcttcaggccgccatccactgcggagccgtacaaatgtacggccagcaacgtcggttcgagatggcgctcgatgacgccaactac
ctctgatagttgagtcgatacttcggcgatcaccgcttccctcatgatgtttaactttgttttagggcgactgccctgctgcgtaacatcgttgctgctc
cataacatcaaacatcgacccacggcgtaacgcgcttgctgcttggatgcccgaggcatagactgtaccccaaaaaaacagtcataacaagcc
atgaaaaccgccactgcgccgttaccaccgctgcgttcggtcaaggttctggaccagttgcgtgagcgcatacgctacttgcattacagcttacg
aaccgaacaggcttatgtccactgggttcgtgccttcatccgtttccacggtgtgcgtcacccggcaaccttgggcagcagcgaagtcgaggcat
ttctgtcctggctggcgaacgagcgcaaggtttcggtctccacgcatcgtcaggcattggcggccttgctgttcttctacggcaaggtgctgtgca
cggatctgccctggcttcaggagatcggaagacctcggccgtcgcggcgcttgccggtggtgctgaccccggatgaagtggttcgcatcctcg
gttttctggaaggcgagcatcgtttgttcgcccagcttctgtatggaacgggcatgcggatcagtgagggtttgcaactgcgggtcaaggatctg
gatttcgatcacggcacgatcatcgtgcgggagggcaagggctccaaggatcgggccttgatgttacccgagagcttggcacccagcctgcgc
gagcaggggaattaattcccacgggttttgctgcccgcaaacgggctgttctggtgttgctagtttgttatcagaatcgcagatccggcttcagccg
gtttgccggctgaaagcgctatttcttccagaattgccatgattttttccccacgggaggcgtcactggctcccgtgttgtcggcagctttgattcgat
aagcagcatcgcctgtttcaggctgtctatgtgtgactgttgagctgtaacaagttgtctcaggtgttcaatttcatgttctagttgctttgttttactggtt
tcacctgttctattaggtgttacatgctgttcatctgttacattgtcgatctgttcatggtgaacagattgaatgcaccaaaaactcgtaaaagctctga
tgtatctatcttttttacaccgttttcatctgtgcatatggacagttttccattgatatgtaacggtgaacagttgttctacttttgtttgttagtcttgatgc
ttcactgatagatacaagagccataagaacctcagatccttccgtatttagccagtatgttctctagtgtggttcgttgtttttgcgtgagccatgagaac
gaaccattgagatcatacttactttgcatgtcactcaaaaattttgcctcaaaactggtgagctgaatttttgcagttaaagcatcgtgtagtgtttttctt
agtccgttatgtaggtaggaatctgatgtaatggttgttggtattttgtcaccattcatttttatctggttgttctcaagttcggttacgagatccatttgtct
atctagttcaacttggaaaatcaacgtatcagtcgggcggcctcgcttatcaaccaccaatttcatattgctgtaagtgtttaaatctttacttattggttt
caaaacccattggttaagccttttaaactcatggtagttattttcaagcattaacatgaacttaaattcatcaaggctaatctctatatttgccttgtgagtt
ttatttgtgttagttatttaataaccactcataaatcctcatagagtatttgttttcaaaagacttaacatgttccagattatattttatgaatttttttaact
ggaaaagataaggcaatatctcttcactaaaaactaattctaatttttcgcttgagaacttggcatagtttgtccactggaaaatctcaaagcctttaacc
aaaggattcctgatttccacagttctcgtcatcagctctctggttgctttagctaatacaccataagcattttccctactgatgttcatcatctgagcgtat
tggttataagtgaacgataccgtccgttattccttgtagggttttcaatcgtggggttgagtagtgccacacagcataaaattagcttggtttcatgct
ccgttaagtcatagcgactaatcgctagttcatttgattgaaaacaactaattcagacatacatctcaattggtctaggtgattttaatcactataccaa
ttgagatgggctagtcaatgataattactagtccttttcctttgagttgtgggtatctgtaaattctgctagacctttgctggaaaacttgtaaattctgct
agaccctctgtaaattccgctagacctttgtgtgttttttttgtttatattcaagtggttataatttatagaataaagaaagaataaaaaaagataaaaaga
atagatcccagccctgtgtataactcactactttagtcagttccgcagtattacaaaaggatgtcgcaaacgctgtttgctcctctacaaaacagacc
ttaaaaccctaaaggcttaagtagcaccctcgcaagctcgggcaaatcgctgaatattccttttgtctccgaccatcaggcacctgagtcgctgtct
ttttcgtgacattcagttcgctgcgctcacggctctggcagtgaatgggggtaaatggcactacaggcgccttttatggattcatgcaaggaaacta
cccataatacaagaaaagcccgtcacgggcttctcagggcgttttatggcgggtctgctatgtggtgctatctgactttttgctgttcagcagttcct
gccctctgattttccagtctgaccacttcggattatcccgtgacaggtcattcagactggctaatgcacccagtaaggcagcggtatcatcaacag
gctta - 3′

### Example 10

### Creation of Upper MVA Pathway Strains REM C8_{—}25, REM C9_{—}25, and REM D1_{—}25 and Control Strains REM D2_{—}25, REM D3_{—}25, and REM D4_{—}25

A host strain CMP865 harboring the atoB deletion locus (loss of endogenous Thiolase activity) as well as a set of previously described mutations shown to support high level MVA production was used to generate the test and controls strains described here. To generate CMP865, a P1 lysate of CMP646 (containing BL21 pgl+PL.2 mKKDyI GI 1.2 gltA ML ackA-pta ldhA attB::Cm) was made and was used in a transduction reaction on strain CMP856, thereby removing the lower mevalonate pathway (e.g. mevalonate kinase, phosphomevalonate kinase, diphosphomevalonate decarboxylase, and isopentenyl diphosphate isomerase) from the chromosome of that strain. The transduction reaction was plated on LB+chloramphenicol 5 ug/ml and one colony was picked and named CMP859. The kanamycin (in atoB locus) and chloramphenicol markers (in attB locus) were looped out concurrently by electroporationg pCP20 (Datsenko and Wanner. 2000. PNAS 97:6640-5) in CMP859, selecting for carbenicillin 50 mg/L-resistant colonies at 30 C, then streaking two transformants at 42 C. A kanamycin-sensitive, chloramphenicol-sensitive colony was selected and named CMP865 (BL21 PL.2mKKDyI GI 1.2 gltA ML atoB attB).

Control strains REM D2_{—}25, REM D3_{—}25, D4_{—}25 were generated by introducing pMCM1221 into strain CMP865 and selecting on LB media containing 50 ug/ml spectinomycin (Teknova, Hollister, Calif.) using a standard electroporation protocol and the Bio-Rad Gene Pulser cuvettes and electroporation system detailed above. From the resulting spectinomycin resistant colonies, 3 were chosen for further analysis and are now referred to as strains REM D2_{—}25, REM D3_{—}25, and REM D4_{—}25. The upper MVA only test strains REM C8_{—}25, REM C9_{—}25, and REM D1_{—}25 were generated in an identical fashion to that just described for the control strains, with the exception that plasmid construct nphT7 with S suis HMGRS/pCL was introduced into the CMP865 host.

### Example 11

### MVA Production from Upper MVA Only Strains REM C8_{—}25, REM C9_{—}25, and REM D1_{—}25 and Control Strains REM D2_{—}25, REM D3_{—}25, and REM D4_{—}25

The MVA titers produced from the MVA only test strains REM C8_{—}25, REM C9_{—}25, and REM D1_{—}25 and control strains REM D2_{—}25, REM D3_{—}25, D4_{—}25 over the course of a 22 hour production period following IPTG-mediated induction of the upper MVA pathway genes. Briefly, control and test strains were grown in 4 ml 1% glucose 0.025% yeast extract TM3 media at 34° C. and induced with 200 uM IPTG at time zero. MVA was measured from cell-free supernatant. Control strains expressing the MVA pathway components from pMCM1221 generated notable MVA titers compared to the test strains. MVA was detected at low levels from 2 of the 3 test strains. It is notable that MVA was detected in these atoB (thoilase) deletion strains thereby establishing that nphT7 was functional within the E. coli BL21 host.

### Example 12

### Creation of Full MVA Pathway, Isoprene Producing Strains REM F7_{—}25, REM F8_{—}25, and REM F9_{—}25 and the IspS Only Control Strain REM F3_{—}25

The E. coli BL21 strain CMP861 (see Example 3) was used as a host strain. The host strain CMP861 is the same background used to generate the previously described MCM1684 and MCM1685 strains which utilize the upper MVA pathway enzymes encoded by nphT5, nphT6, and nphT7 genes derived from Streptomyces sp. strain CL190 to produce isoprene at an enhanced level over that offered by the endogenous DXP pathway of E. coli. Plasmid construct pDW240 (pTrc P. alba IspS MEA-mMVK (Carb50)), carries an IPTG-inducible ispS (Isoprene Synthase) variant and a carbenicillin resistance gene. Briefly, the full MVA pathway, isoprene producing test strains REM F7_{—}25, REM F8_{—}25, and REM F9_{—}25 were generated by introducing plasmid construct nphT7 with S suis HMGRS/pCL together with plasmid construct pDW240 into strain CMP861 and selecting on LB media containing 50 ug/ml spectinomycin and 50 ug/ml carbenicillin (Teknova, Hollister, Calif.) using a standard electroporation protocol and the Bio-Rad Gene Pulser cuvettes and electroporation system detailed above. From the resulting spectinomycin and carbenicillin resistant colonies, 3 were chosen for further analysis and are now referred to as strains REM F7_{—}25, REM F8_{—}25, and REM F9_{—}25. Similarly, the IspS alone control strain was generated by introducing the pDW240 plasmid construct alone into strain CMP861 via electroporation and selecting on LB media containing only 50 ug/ml carbenicillin (Teknova, Hollister, Calif.). One carbenicillin resistant colony was chosen to serve as a control strain in subsequent experiments and was named REM F3_{—}25. The isoprene produced by the IspS alone control strain REM F3_{—}25 reflects the endogenous level of the IspS substrate (DMAPP) the DXP pathway of E. coli supports under the growth conditions assessed.

### Example 13

### Isoprene Production from Full MVA Pathway Only Test Strains REM F7_{—}25, REM F8_{—}25, and REM F9_{—}25, Previously Described MCM1684 and MCM1685 NphT7-Utilizing Strains, and the IspS Alone Control Strain

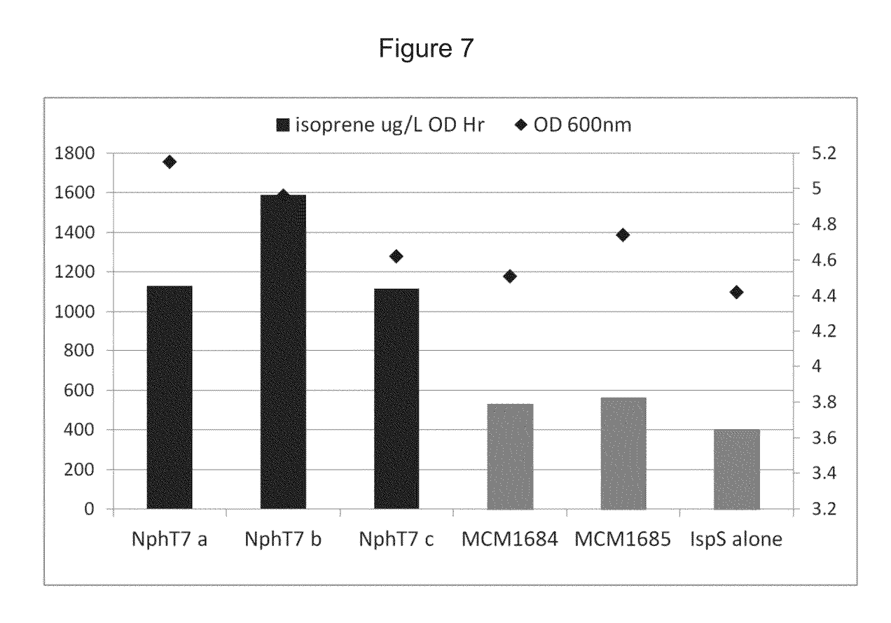

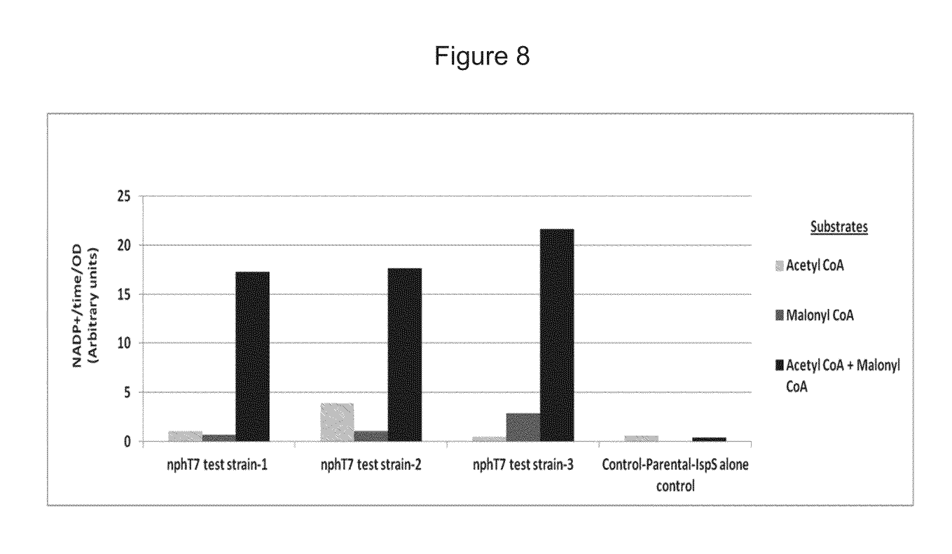

Shown in FIG. 7 is the specific productivity of isoprene (ug/L OD Hr) calculated from the optical density (OD) and level of isoprene measured for each culture of the full MVA pathway only test strains REM F7_{—}25, REM F8_{—}25, and REM F9_{—}25 (represented as strains NphT7 a-c in FIG. 8 respectively), the previously described MCM1684 and MCM1685 NphT7-utilizing strains, and the IspS alone control strain after a 3.5 hour growth period following IPTG-mediated induction of relevant gene expression. Briefly, control and test strains were grown in 20 ml 1% glucose 0.05% yeast extract TM3 media at 34° C. and induced with 200 uM IPTG at time zero. Isoprene and OD measurements were performed essentially as described before, as was calculation of the specific productivities of isoprene reported in FIG. 7. Cell pellets from 18 ml of each of the aforementioned cultures were generated 5.5 hours after IPTG-induction and subsequently analyzed for upper MVA pathway and IspS activities (see below). As previously observed, the MCM1684 and MCM1685 NphT7-utilizing strains expressing all 3 upper MVA pathway components derived from Streptomyces sp. strain CL190 could support a modestly higher specific productivity of isoprene than an IspS alone control strain. Interestingly, the newly created test strains REM F7_{—}25, REM F8_{—}25, and REM F9_{—}25 described here generated roughly 3-fold higher levels of isoprene than the previously characterized MCM1684 and MCM1685 strains. This data again supports the idea that NphT7 is functional within the E. coli BL21 host. This data also suggests that the plasmid construct nphT7 with S suis HMGRS/pCL encoding NphT7 Acetoacetyl-CoA Synthase together with the Streptococcus suis HMG-CoA Synthase and HMG-CoA Reductase produces a more active upper MVA pathway than the previously described 3 component pathway derived solely from Streptomyces sp. strain CL190 genes which strains MCM1684 and MCM1685 express.

### Example 14

### Catalytic Activity Assays for Acetoacetyl-CoA Synthase (NphT7) Strains

### Materials:

Acetyl-CoA, malonyl-CoA, NADPH, TRIS base, AEBSF, DNAase, lysozyme, sodium chloride, and magnesium chloride were purchased from Sigma. DMAPP was chemically synthesized.

### Cell Growth and Lysate Preparation.

Cells were grown at 34° C. in TM3 media containing 1% glucose, 0.05% yeast extract and 200 μM IPTG for 5.5 hrs. Cells were then centrifuged at 5000 RPM for 15 minutes at 4° C. in an Eppendorf 5804R centrifuge. Cell pellets were resuspended in a solution containing 100 mM Tris, 100 mM NaCl, 0.5 mM AEBSF, 1 mg/ml lysozyme, 0.1 mg/ml DNAase, pH 7.6. The cell suspension was lysed using a french pressure cell at 14,000 psi. The lysate was then centrifuged at 15,000 RPM for 10 minutes at 4° C. in an Eppendorf 5804R centrifuge. The supernatant was collected for enzyme activity assays.

(A) Coupled Acetoacetyl-CoA Synthase (NphT7), HMG-CoA synthase, HMG-CoA Reductase Catalytic Activity Assay.

Cell lysate acetyl-CoA and malonyl-CoA activity assays were conducted with 1 mM acetyl-CoA, 1 mM malonyl-CoA, or both, and 0.4 mM NADPH, 100 mM Tris, 100 mM NaCl, pH 7.6 and 20 μl of clarified cell lysate. Reactions were initiated by the addition of acetyl-CoA, malonyl-CoA or both. NADPH oxidation was monitored in a 96-well plate at 340 nm using a SpectraMax Plus190 (Molecular Devices, Sunnyvale, Calif.). All reactions were conducted at 25° C. in a final volume of 100 μl. The oxidation rate of NADPH in the absence of acetyl-CoA or malonyl-CoA was subtracted from reaction rates in the presence of acetyl-CoA, malonyl-CoA or both.

### (B) Isoprene Synthase Catalytic Activity Assay

Twenty five μL of E. coli lysate were incubated with 5 mM DMAPP, 50 mM MgCl2, and 100 mM Tris/NaCl, pH 7.6 at 34° C. in a total volume of 100 μL in a 2 mL gas-tight vial for 15 minutes. Reactions were terminated by the addition of 100 μL of 250 mM EDTA. The glass vials were analyzed by GC-MS to determine the concentration of isoprene generated in the reactions.

### Results:

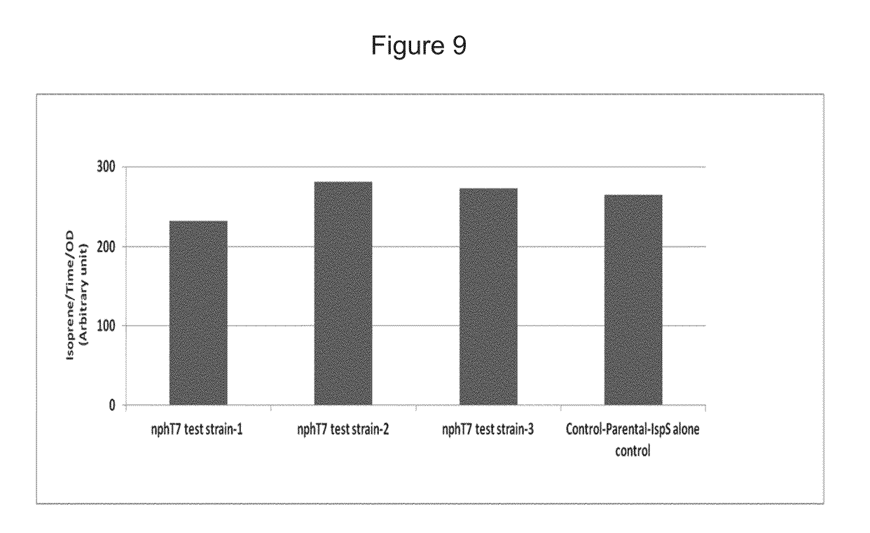

In these studies, the oxidation of NADPH was monitored in the presence of cell lysate and acetyl-CoA, malonyl-CoA or both acetyl-CoA and malonyl-CoA. The results indicate that the rate of oxidation of NADPH in acetoacetyl-CoA Synthase (nphT7) containing strains is the greatest in the presence of acetyl-CoA and malonyl-CoA (see FIG. 8). The rate of NADPH oxidation in control strains lacking acetoacetyl-CoA Synthase (nphT7) was decreased compared to strains containing acetoacetyl-CoA Synthase (nphT7) and the rate of oxidation of control strains was not dependent on the presence of acetyl-CoA or malonyl-CoA (FIG. 8). These results are consistent with composite acetoacetyl-CoA Synthase (nphT7), HMG-CoA synthase, and HMG-CoA reductase activities being dependent on the presence of malonyl-CoA and acetyl-CoA. HMG-CoA synthase catalytic activity is dependent on the presence of acetyl-CoA, therefore, one can conclude that the acetoacetyl-CoA Synthase (nphT7) activity requires the presence of malonyl-CoA. These results clearly support acetoacetyl-CoA Synthase (nphT7) utilization of both malonyl-CoA as a substrate in the production of acetoacetyl-CoA. Isoprene synthase activity was assayed to ensure that differences in isoprene specific productivity (see FIG. 7) were not due to differences in isoprene synthase activity (FIG. 9).

### Example 15

### Construction of Amorphadiene- or Farnesene-Producing Strains

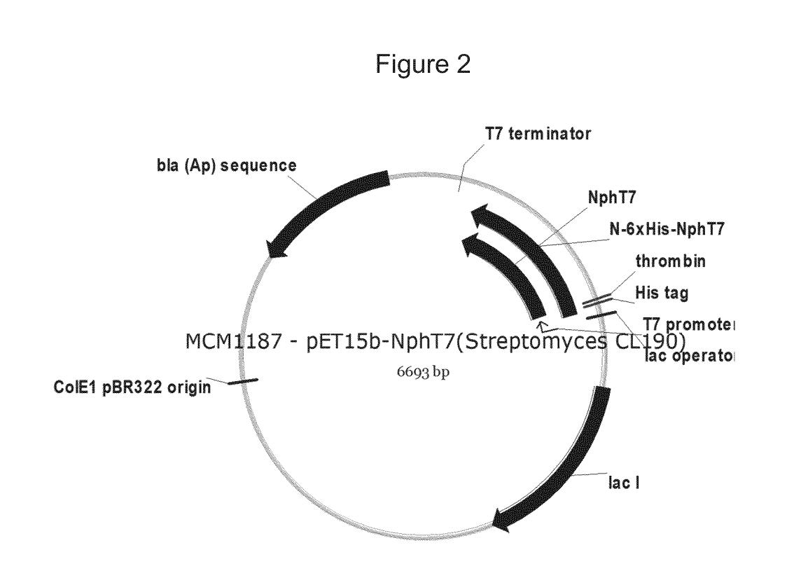

A lower mevalonate pathway is introduced by transduction into MCM1684 using a lysate from MCM521. The kanamycin marker is looped out according to the manufacturer (Gene Bridges, Heidelberg, Germany). The lower pathway from MCM521 can be modified by changing the promoter upstream of the operon by modifying the rbs in front of each gene via the use of alternative genes. Farnesyl diphosphate synthase (ispA) is overexpressed, either by altering the promoter and/or rbs on the chromosome, or by expressing it from a plasmid. Plasmid pMCM1321 is co-electroporated with a variation of plasmid pDW34 (See U.S. Patent Application Publication No: 2010/0196977; FIG. 2). The plasmids which are variants of pDW34 contain the farnesene synthase codon optimized for E. coli or amorphadiene synthase codon optimized for E. coli, instead of isoprene synthase. Colonies are selected on LB+spectinomycin 50 ug/mL+carbenicillin 50 ug/mL.

### Example 16

### Production of Amorphadiene or Farnesene in Strains Containing the Plasmids with Acetoactetyl-CoA Synthase

### (i) Materials

TM3 media recipe (per liter fermentation media): K2HPO4 13.6 g, KH2PO4 13.6 g, MgSO4*7H2O 2 g, citric acid monohydrate 2 g, ferric ammonium citrate 0.3 g, (NH4)2SO4 3.2 g, yeast extract 0.2 g, 1000× Trace Metals Solution 1 ml. All of the components are added together and dissolved in diH2O. The pH is adjusted to 6.8 with ammonium hydroxide (30%) and brought to volume. Media is then filter-sterilized with a 0.22 micron filter. Glucose 10.0 g and antibiotics are added after sterilization and pH adjustment.

1000× Trace Metal Solution (per liter fermentation media): Citric Acid*H2O 40 g, MnSO04*H2O 30 g, NaCl 10 g, FeSO4*7H2O 1 g, CoCl2*6H2O 1 g, ZnSO4*7H₂O 1 g, CuSO4*5H2O 100 mg, H3BO3 100 mg, NaMoO4*2H2O 100 mg. Each component is dissolved one at a time in diH2O. The pH is adjusted to 3.0 with HCl/NaOH, and then the solution is brought to volume and filter-sterilized with a 0.22 micron filter.

### (ii) Experimental Procedure

Cells are grown overnight in Luria-Bertani broth+antibiotics. The day after, they are diluted to an OD600 of 0.05 in 20 mL TM3 medium containing 50 ug/ml of spectinomycin and 50 ug/mL carbenicillin (in a 250-mL baffled Erlenmeyer flask), and incubated at 34° C. and 200 rpm. Prior to inoculation, an overlay of 20% (v/v) dodecane (Sigma-Aldrich) is added to each culture flask to trap the volatile sesquiterpene product as described previously (Newman et. al., 2006).

After 2 h of growth, OD600 is measured and 0.05-0.40 mM isopropyl β-d-1-thiogalactopyranoside (IPTG) is added. Samples are taken regularly during the course of the fermentation. At each timepoint, OD600 is measured. Also, amorphadiene or farnesene concentration in the organic layer is assayed by diluting the dodecane overlay into ethyl acetate. Dodecane/ethyl acetate extracts are analyzed by GC-MS methods as previously described (Martin et. al., Nat. Biotechnol. 2003, 21:96-802) by monitoring the molecular ion (204 m/z) and the 189 m/z fragment ion for amorphadiene or the molecular ion (204 m/z) for farnesene. Amorphadiene or farnesene samples of known concentration are injected to produce standard curves for amorphadiene or farnesene, respectively. The amount of amorphadiene or farnesene in samples is calculated using the amorphadiene or farnesene standard curves, respectively.

### (iii) Results

When the strains containing pMCM1321 are compared to the same background without the acetoacetyl-CoA synthase gene, increased specific productivity, yield, CPI and/or titer of amorphadiene or farnesene are observed.

### (iv) References

Newman, J. D., Marshal, J. L., Chang, M. C. Y., Nowroozi, F., Paradise, E. M., Pitera, D. J., Newman, K. L., Keasling, J. D., 2006. High-level production of amorpha-4,11-diene in a two-phase partitioning bioreactor of metabolically engineered E. coli. Biotechnol. Bioeng. 95:684-691.
Martin, V. J., Pitera, D. J., Withers, S. T., Newman, J. D., Keasling, J. D., 2003. Engineering a mevalonate pathway in E. coli for production of terpenoids. Nat. Biotechnol. 21:796-802.

What is claimed is:

1. A recombinant microorganism capable of producing isoprene comprising one or more nucleic acids encoding a polypeptide capable of synthesizing acetoacetyl-CoA from malonyl-CoA and acetyl-CoA and one or more nucleic acids encoding:
a. an isoprene synthase polypeptide, wherein the isoprene synthase polypeptide is encoded by a heterologous nucleic acid; and
b. one or more mevalonate (MVA) pathway polypeptides,
wherein culturing of said recombinant microorganism in a suitable media provides for the production of said polypeptides and synthesis of isoprene.

2. The recombinant microorganism according to claim 1, wherein the one or more nucleic acids encoding a polypeptide capable of synthesizing acetoacetyl-CoA from malonyl-CoA and acetyl-CoA is an acetoacetyl-CoA synthase gene.

3. The recombinant microorganism according to claim 2, wherein the acetoacetyl-CoA synthase gene is a gene from an actinomycete.

4. The recombinant microorganism according to claim 3, wherein the acetoacetyl-CoA synthase gene is from the genus Streptomyces.

5. The recombinant microorganism according to claim 4, wherein the acetoacetyl-CoA synthase gene encodes a protein having the amino acid sequence of SEQ ID NO: 1 or a protein having an amino acid sequence with an 80% or more identity to the amino acid sequence of SEQ ID NO: 1 and having a function of synthesizing acetoacetyl-CoA from malonyl-CoA and acetyl-CoA.

6. The recombinant microorganism of claim 1, wherein the isoprene synthase polypeptide is a plant isoprene synthase polypeptide or a variant thereof.

7. The recombinant microorganism of claim 6, wherein the isoprene synthase polypeptide is a polypeptide from Pueraria or Populus or a hybrid, Populus alba×Populus tremula or a variant thereof.

8. The recombinant microorganism of claim 7, wherein the isoprene synthase polypeptide is selected from the group consisting of Pueraria montana or Pueraria lobata, Populus tremuloides, Populus alba, Populus nigra, and Populus trichocarpa or a variant thereof.

9. The recombinant microorganism of claim 1, wherein said one or more nucleic acids encoding one or more MVA pathway polypeptides of (b) is a heterologous nucleic acid.

10. The recombinant microorganism of claim 1, wherein said one or more nucleic acids encoding more MVA pathway polypeptides of (b) is a copy of an endogenous nucleic acid.

11. The recombinant microorganism of claim 1, wherein the one or more MVA pathway polypeptides is selected from (a) an enzyme that condenses acetoacety-CoA with acety-CoA to form HMG-Co-A; (b) an enzyme that converts HMG-CoA to mevolonate; (c) an enzyme that phosphorylates mevalonate to mevalonate 5-phosphate; (d) an enzyme that converts mevalonate 5-phosphate to mevalonate 5-pyrophosphate; and (e) an enzyme that converts mevalonate 5-pyrophosphate to isopentenyl pyrophosphate.

12. The recombinant microorganism of claim 1, wherein the enzyme that phosphorylates mevalonate to mevalonate 5-phosphate is selected from the group consisting of M. mazei mevalonate kinase, M. burtonii mevalonate kinase polypeptide, Lactobacillus mevalonate kinase polypeptide, Lactobacillus sakei mevalonate kinase polypeptide, yeast mevalonate kinase polypeptide, Saccharomyces cerevisiae mevalonate kinase polypeptide, Streptococcus mevalonate kinase polypeptide, Streptococcus pneumoniae mevalonate kinase polypeptide, and Streptomyces mevalonate kinase polypeptide, or Streptomyces CL190 mevalonate kinase polypeptide.

13. The recombinant microorganism of claim 12, wherein the enzyme that phosphorylates mevalonate to mevalonate 5-phosphate is M. mazei mevalonate kinase.

14. The recombinant microorganism of claim 1, further comprising one or more nucleic acids encoding an isopentenyl-diphosphate delta-isomerase (IDI) polypeptide

15. The recombinant microorganism of claim 1, further comprising one or more nucleic acids encoding one or more 1-deoxy-D-xylulose-5-phosphate (DXP) pathway polypeptides.

16. The recombinant microorganism of claim 15, wherein said one or more nucleic acids encoding one or more DXP pathway polypeptides of is a heterologous nucleic acid encoding.

17. The recombinant microorganism of claim 15, wherein said one or more nucleic acids encoding one or more DXP pathway polypeptides is a copy of an endogenous nucleic acid.

18. The recombinant microorganism of claim 15, wherein the one or more DXP pathway polypeptides is selected from (a) 1-deoxy-D-xylulose-5-phosphate synthase (DXS), (b) 1-deoxy-D-xylulose-5-phosphate reductoisomerase (DXR), (c) 4-diphosphocytidyl-2C-methyl-D-erythritol synthase (MCT), (d) 4-diphosphocytidyl-2-C-methyl-D-erythritol kinase (CMK), (e) 2C-methyl-D-erythritol 2,4-cyclodiphosphate synthase (MCS), (f) 1-hydroxy-2-methyl-2-(E)-butenyl 4-diphosphate synthase (HDS), and (g) 1-hydroxy-2-methyl-2-(E)-butenyl 4-diphosphate reductase (HDR).

19. The recombinant microorganism of claim 18, wherein the DXP pathway polypeptide is DXS.

20. The recombinant microorganism of claim 1, wherein the one or more heterologous nucleic acids is placed under an inducible promoter or a constitutive promoter.

21. The recombinant microorganism of claim 1, wherein the one or more heterologous nucleic acids is cloned into one or more multicopy plasmids.

22. The recombinant microorganism of claim 1, wherein the one or more heterologous nucleic acids is integrated into a chromosome of the cells

23. The recombinant microorganism of claim 1, wherein the microorganism is a bacterial, algal, fungal or yeast cell.

24. The recombinant microorganism of claim 23, wherein the microorganism is a bacterial cell.

25. The bacterial cell of claim 24, wherein the bacterial cell is a gram-positive bacterial cell or gram-negative bacterial cell.

26. The bacterial cell of claim 25, wherein the bacterial cell is selected from the group consisting of E. coli, L. acidophilus, Corynebacterium sp., P. citrea, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., and P. alcaligenes cells.

27. The bacterial cell of claim 26, wherein the bacterial cell is an E. coli cell.

28. The recombinant microorganism of claim 23, wherein the microorganism is an algal cell.

29. The recombinant microorganism of claim 23, wherein the microorganism is a fungal cell.

30. The recombinant microorganism of claim 23, wherein the microorganism is a yeast cell.

31. The yeast cell of claim 30, wherein the yeast cell is selected from the group consisting of Saccharomyces sp., Schizosaccharomyces sp., Pichia sp., or Candida sp.

32. The yeast cell of claim 31, wherein the yeast cell is a Saccharomyces cerevisiae cell.

33. A recombinant microorganism capable of producing an isoprenoid comprising one or more nucleic acids encoding a polypeptide capable of synthesizing acetoacetyl-CoA from malonyl-CoA and acetyl-CoA and one or more nucleic acids encoding:
a. one or more nucleic acids encoding a polyprenyl pyrophosphate synthase; and
b. one or more nucleic acids encoding one or more mevalonate (MVA) pathway polypeptides,
wherein culturing of said recombinant microorganism in a suitable media provides for production of said polypeptides and synthesis of a recoverable amount of isoprenoid.

34. The recombinant microorganism of claim 33, wherein said one or more nucleic acids encoding one or more MVA pathway polypeptides of (b) is a heterologous nucleic acid.

35. The recombinant microorganism of claim 33, wherein the one or more MVA pathway polypeptides is selected from the group consisting of (a) an enzyme that condenses acetoacety-CoA with acety-CoA to form HMG-Co-A; (b) an enzyme that converts HMG-CoA to mevolonate; (c) an enzyme that phosphorylates mevalonate to mevalonate 5-phosphate; (d) an enzyme that converts mevalonate 5-phosphate to mevalonate 5-pyrophosphate; and (e) an enzyme that converts mevalonate 5-pyrophosphate to isopentenyl pyrophosphate.

36. The recombinant microorganism of claim 33, wherein the enzyme that phosphorylates mevalonate to mevalonate 5-phosphate is selected from the group consisting of M. mazei mevalonate kinase, M. burtonii mevalonate kinase polypeptide, Lactobacillus mevalonate kinase polypeptide, Lactobacillus sakei mevalonate kinase polypeptide, yeast mevalonate kinase polypeptide, Saccharomyces cerevisiae mevalonate kinase polypeptide, Streptococcus mevalonate kinase polypeptide, Streptococcus pneumoniae mevalonate kinase polypeptide, and Streptomyces mevalonate kinase polypeptide, Streptomyces CL190 mevalonate kinase polypeptide.

37. The recombinant microorganism of claim 36, wherein the enzyme that phosphorylates mevalonate to mevalonate 5-phosphate is M. mazei mevalonate kinase.

38. The recombinant microorganism of claim 33, further comprising one or more nucleic acids encoding an isopentenyl-diphosphate delta-isomerase (IDI) polypeptide

39. The recombinant microorganism of claim 33, wherein the one or more heterologous nucleic acids is placed under an inducible promoter or a constitutive promoter.

40. The recombinant microorganism of claim 33, wherein the one or more heterologous nucleic acids is cloned into one or more multicopy plasmids.

41. The recombinant microorganism of claim 33, wherein the one or more heterologous nucleic acids is integrated into a chromosome of the cells

42. The recombinant microorganism of claim 33, wherein the microorganism is a bacterial, algal, fungal or yeast cell.

43. The recombinant microorganism of claim 42, wherein the microorganism is a bacterial cell.

44. The bacterial cell of claim 43, wherein the bacterial cell is a gram-positive bacterial cell or gram-negative bacterial cell.

45. The bacterial cell of claim 44, wherein the bacterial cell is an E. coli cell.

46. The recombinant microorganism of claim 42, wherein the microorganism is an algal cell.

47. The recombinant microorganism of claim 42, wherein the microorganism is a fungal cell.

48. The recombinant microorganism of claim 42, wherein the microorganism is a yeast cell.

49. The yeast cell of claim 48, wherein the yeast cell is a Saccharomyces cerevisiae cell.

50. The recombinant microorganism of claim 33, wherein the isoprenoid is selected from group consisting of monoterpenes, diterpenes, triterpenes, tetraterpenes, sequiterpene, and polyterpene.

51. The recombinant microorganism of claim 50, wherein the isoprenoid is a sesquiterpene.

52. The recombinant microorganism of claim 33, wherein the isoprenoid is selected from the group consisting of abietadiene, amorphadiene, carene, farnesene, α-farnesene, β-farnesene, farnesol, geraniol, geranylgeraniol, linalool, limonene, myrcene, nerolidol, ocimene, patchoulol, β-pinene, sabinene, γ-terpinene, terpindene and valencene.

53. A method of producing isoprene, the method comprising:
a. culturing a recombinant microorganism comprising one or more nucleic acids encoding (i) a polypeptide capable of synthesizing acetoacetyl-CoA from malonyl-CoA and acetyl-CoA and one or more nucleic acids encoding: (ii) an isoprene synthase polypeptide, wherein the isoprene synthase polypeptide is encoded by a heterologous nucleic acid; and (iii) one or more mevalonate (MVA) pathway polypeptides, and
b. producing isoprene.

54. The method of claim 53, further comprising recovering the isoprene produced by the recombinant microorganism.

55. The method of claim 53, wherein the or more nucleic acids encoding a polypeptide capable of synthesizing acetoacetyl Co-A from malonyl Co-A and acetyl-CoA is an acetoacetyl-CoA synthase gene.

56. The method of claim 53, wherein the isoprene synthase polypeptide is a plant isoprene synthase polypeptide.

57. The method of claim 53, wherein the one or more MVA pathway polypeptides is selected from the group consisting of (a) an enzyme that condenses acetoacety-CoA with acety-CoA to form HMG-Co-A; (b) an enzyme that converts HMG-CoA to mevolonate; (c) an enzyme that phosphorylates mevalonate to mevalonate 5-phosphate; (d) an enzyme that converts mevalonate 5-phosphate to mevalonate 5-pyrophosphate; and (e) an enzyme that converts mevalonate 5-pyrophosphate to isopentenyl pyrophosphate.

58. The method of claim 53, further comprising one or more nucleic acids encoding an isopentenyl-diphosphate delta-isomerase (IDI) polypeptide.

59. The method of claim 53, wherein said recombinant microorganism further comprises one or more nucleic acids encoding one or more 1-deoxy-D-xylulose-5-phosphate (DXP) pathway polypeptides.

60. The method of claim 53, wherein the microorganism is a bacterial, algal, fungal or yeast cell.

61. The method of claim 60, wherein the microorganism is a bacterial cell.

62. The method of claim 61, wherein the bacterial cell is a gram-positive bacterial cell or gram-negative bacterial cell.

63. The method of claim 62, wherein the bacterial cell is an E. coli cell.

64. The method of claim 60, wherein the microorganism is a yeast cell.

65. The method of claim 64, wherein the yeast cell is a Saccharomyces cerevisiae cell.

66. A method of producing an isoprenoid, the method comprising:
a. culturing a recombinant microorganism comprising one or more nucleic acids encoding (i) a polypeptide capable of synthesizing acetoacetyl-CoA from malonyl-CoA and acetyl-CoA and one or more nucleic acids encoding: (ii) a polyprenyl pyrophosphate synthase polypeptide, wherein the polyprenyl pyrophosphate synthase polypeptide is encoded by a heterologous nucleic acid; and (iii) one or more mevalonate (MVA) pathway polypeptides, and
b. producing said isoprenoid.
